(19) United States
(12) Patent Application Publication
(10) Pub. No.: US 2009/0291945 A1
(43) Pub. Date: Nov. 26, 2009

(54) CYSTEINE PROTEASE INHIBITORS

(75) Inventors: Gen Unoki, Tokyo (JP); Takashi Hayamizu, Tokyo (JP); Hiroshi Eguchi, Tokyo (JP); Yumiko Muroga, Tokyo (JP); Toshiyuki Kaneko, Tokyo (JP); Naoki Yajima, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: Teijin Pharma Limited, Tokyo (JP)

(21) Appl. No.: 12/419,722

(22) Filed: Apr. 7, 2009

Related U.S. Application Data
(60) Provisional application No. 61/043,412, filed on Apr. 9, 2008.

(30) Foreign Application Priority Data
Apr. 9, 2008 (JP) 2008-101461

(51) Int. Cl. A61K 31/54; A61K 31/535; A61K 31/445; A61K 31/165; C07D 279/12; C07D 265/28; C07D 211/60; C07C 237/02

(52) U.S. Cl. 514/227.5; 514/231.2; 514/330; 514/620; 544/59; 544/98; 546/227; 564/164

(57) ABSTRACT

To provide a compound having an excellent cysteine protease inhibitory effect, and to provide a drug for treatment or prevention of the disease selected from the group consisting of osteoporosis, osteoarthritis, chronic rheumatoid arthritis, Paget's disease of bone, hypercalcemia, bone metastasis of cancer, and ostealgia. A compound represented by formula (1) or a pharmaceutically acceptable salt thereof, or a drug or pharmaceutical composition containing the same as an effective component.

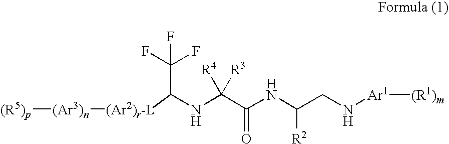

### TECHNICAL FIELD

The present invention relates to a novel compound having a cysteine protease inhibitory activity (especially cathepsin K inhibitory activity), production method thereof and a cysteine protease inhibitor (especially cathepsin K inhibitor) containing the compound as an active ingredient. Specifically, the present invention relates to a compound useful for treatment or prevention of osteoporosis, osteoarthritis, chronic rheumatoid arthritis, Paget's disease of bone, hypercalcemia, bone metastasis of cancer, or ostealgia.

### BACKGROUND ART

In recent years, associated with the rapid progress toward an aging society, ever-increasing number of bedridden elderly people is causing serious social and economical problems. As the major causes of being bedridden, cerebral stroke, senility, and bone fracture resulting from osteoporosis are mentioned. Especially it is pointed out that, because it frequently takes so long time to heal the bone fracture in the advanced age, the physical strength during the cure is significantly decreased and the probability of becoming bedridden is high. Therefore, prevention and/or treatment of this state is an important issue in order to maintain and improve the QOL (quality of life) of the elderly people.

Clinical state of osteoporosis is characterized by decreasing bone strength and increasing risk of bone fracture according to the change of fine structure of bone tissue caused by the decrease in bone mass. Bone tissue is consistently repeating remodeling in the organism by interaction of bone formation by osteoblasts of mesenchymal system and bone resorption by osteoclasts of hematopoietic system, the balance of which maintains the bone mass. It is considered that osteoporosis is caused by the failure of this balance for some reason and continuation of the state in which bone resorption exceeds bone formation for a long period. Since the increase of bone resorption closely relates to the pathogenesis and progression of the disease state, a bone resorption inhibitor is generally used in a drug therapy for osteoporosis. However, a pharmaceutical agent having a bone resorption inhibitory effect such as a calcitonin preparation, an estrogen preparation, a vitamin K preparation, a bisphosphonate preparation, and the like, which is currently used, has a problem in its curing effect, an immediate effectivity, an adverse effect, dose compliance, and the like. Therefore, development of the bone resorption inhibitor which may become a more effective treatment or prevention drug for osteoporosis is desired.

Osteoclasts, which are multinucleate giant cells originated mainly from hematopoietic stem cells, play a role of bone resorption. Cells of monocyte-macrophage lineage differentiate to osteoclast precursors by the action of various cytokines and the like. Then the precursors become mononucleate preosteoclasts, which are drawn to the bone surface, and are fixed and multinucleated to become osteoclasts. The differentiated osteoclasts, when activated, surround the bone surface with ruffled border consisting of complexed cytoplasmic processes, dissolve hydroxyapatite by releasing acid, and digest protein matrix such as collagen type I by secreting various proteases. It is considered that the proteases involved in the digestion of collagen are the essential components for bone metabolic turnover and occurrence and progression of osteoporosis, because about 95% of the organic matrix of bone is collagen. As the major proteases involved in the matrix digestion by osteoclasts, cysteine proteases are mentioned, among which involvement of cathepsin family belonging to papain superfamily is widely known. Especially there are many reports regarding the relationship of cathepsin K and various pathological states, which is considered as potential drug target.

Cathepsin K is also referred to as cathepsin O, cathepsin O2, and cathepsin X and is one of the enzymes belonging to cysteine cathepsin family that is part of a papain superfamily of a cysteine protease. As the enzymes classified in cysteine proteases in the cathepsin family, cathepsin B, cathepsin C, cathepsin F, cathepsin H, cathepsin L, cathepsin O, cathepsin S, cathepsin V (also referred to as L2), cathepsin W, and cathepsin Z (also referred to as cathepsin X) are further known. Cathepsin K shows a high level expression in normal osteoclasts and is reported to be a major cysteine protease in these cells (Non-patent Document 1 to 3). Further, in view of the finding that the cathepsin K gene is mutated in dwarfism patients whose cause is considered to be abnormal bone resorption, and the like, it is suggested that cathepsin K is essential in the function of osteoclasts (Non-patent Document 4). Therefore, effective remedy is expected for the disease resulting from excessive bone resorption, such as osteoporosis, by selective inhibition of cathepsin K. In fact, clinical trials have been conducted for some drugs which selectively inhibit cathepsin K and there are some reports showing the curing effect of these drugs (Non-patent Documents 5 and 6).

It is considered that selective inhibition of cathepsin K is also useful for treatment of other diseases. Such diseases include autoimmune disease (such as chronic rheumatoid arthritis), osteoarthritis, Paget's disease of bone, hypercalcemia, bone metastasis of cancer, or ostealgia. For example, cathepsin K is expressed in synovial membrane and synovial bone destruction site of chronic rheumatoid arthritis patients (Non-patent Document 7-9), and the inhibitory substances showed a drug efficacy in disease model animals (Non-patent Document 10 and 11). The expression level of cathepsin K is increased in synovial membrane and cartilage surface of osteoarthritis (Non-patent Document 12-14). Expression of cathepsin K is recognized in various cancer cells (Non-patent Document 15-19), and relationship with bone metastasis has been shown (Non-patent Document 20 and 21). In addition, it is considered that selective inhibition of cathepsin K is useful for the treatment of disease caused by enhancement of bone resorption activity of osteoclasts, for example, Paget's disease of bone, hypercalcemia, or ostealgia.

For the reasons described above, cathepsin K has come to attract attention as a target molecule for treatment and prevention of disease and research and development of cathepsin K inhibitors are also being performed intensely. So far, as the cathepsin K inhibitor, for example, linear ketone type inhibitors (Non-patent Document 22), a cyclic ketone type inhibitor (Non-patent Document 23-26), an aldehyde type inhibitor (Non-patent Document 27), an α-ketoamide type inhibitor (Non-patent Document 28), N-aryl ethylenediamine type inhibitors (Patent Document 1-3 and Non-patent Document 29, 30, and 34), cyanomethylene type inhibitors (Patent Document 4 and Non-patent Document 31-33), and the like have been reported.

As described above, although compounds which inhibit cathepsin K are attracting attention as bone resorption inhibitors and many derivatives have been reported, no compounds have been put to practical use yet as a therapeutic drug for metabolic bone disease. In addition, the structures of these compounds are different from the structure of the compound of the present invention. Note that an N-aryl ethylenediamine type compound has been reported also as a cathepsin S inhibitor (Patent Document 5).

Especially Patent Document 1 describes a compound represented by the following general formula (A) as a small molecule which inhibits cathepsin K.

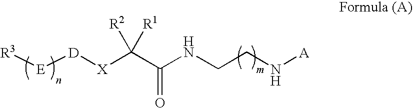

However, in Patent Document 1, only a compound represented by the following formula (B) is described as a specific compound.

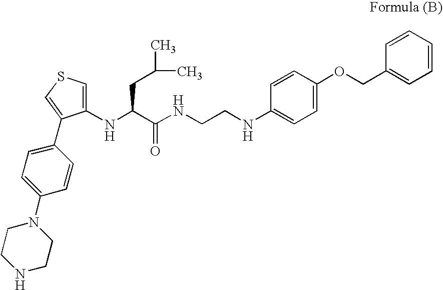

Patent Document 1: WO2002/070517

Patent Document 2: Japanese Patent Laid-open Publication No. 2004-256525

Patent Document 3: WO2000/048993

Patent Document 4: WO2003/075836

Patent Document 5: WO2004/112709

Non-patent Document 1: J. Biol. Chem., 269, 1106 (1994)

Non-patent Document 2: Biochem. Biophys. Res. Commun., 206, 89 (1995)

Non-patent Document 3: FEBS Lett., 357, 129 (1995)

Non-patent Document 4: Science, 273, 1236 (1996)

Non-patent Document 5: 28^{th} ASBMR, Abst 1085

Non-patent Document 6: 29^{th} ASBMR, Abst 1128

Non-patent Document 7: J. Rheumatol., 25, 1887 (1998)

Non-patent Document 8: Am J Pathol., 159, 2167 (2001)

Non-patent Document 9: Arthritis Res Ther., 7, R65-70 (2005)

Non-patent Document 10: J. Bone Miner. Res., 12, 1396 (1997)

Non-patent Document 11: Science., 319, 624 (2008)

Non-patent Document 12: Arthritis Rheum., 42, 1588 (1999)

Non-patent Document 13: Arthritis Rheum., 46, 663 (2002)

Non-patent Document 14: Arthritis Rheum., 46, 953 (2002)

Non-patent Document 15: Cancer Res., 57, 5386 (1997)

Non-patent Document 16: Matrix Biol., 19, 717 (2001)

Non-patent Document 17: Pancreas., 25, 317 (2002)

Non-patent Document 18: J. Bone Miner Res., 18, 222 (2003)

Non-patent Document 19: Am J Clin Pathol., 125, 847 (2006)

Non-patent Document 20: Clin Cancer Res., 9, 295 (2003)

Non-patent Document 21: Mol Carcinog., 47, 66 (2008)

Non-patent Document 22: J. Am. Chem. Soc., 120, 9114-9115 (1998)

Non-patent Document 23: J. Med. Chem., 41, 3563-3567 (1998)

Non-patent Document 24: J. Med. Chem., 44, 1380-1395 (2001)

Non-patent Document 25: Bioorg. Med. Chem., 12, 5689-5710 (2004)

Non-patent Document 26: J. Med. Chem., 49, 1597-1612 (2006)

Non-patent Document 27: Bioorg. Med. Chem. Letters., 14, 275-278 (2004)

Non-patent Document 28: Bioorg. Med. Chem. Letters., 15, 3540-3546 (2005)

Non-patent Document 29: J. Med. Chem., 45, 2352-2354 (2002)

Non-patent Document 30: Bioorg. Med. Chem., 14, 6789-6806 (2006)

Non-patent Document 31: J. Med. Chem., 46, 3709-3727 (2003)

Non-patent Document 32: Bioorg. Med. Chem. Lett., 14, 4291-4295 (2004)

Non-patent Document 33: J. Med. Chem., 49, 1066-1079 (2006)

Non-patent Document 34: Bioorg. Med. Chem. Lett., 14, 87-90 (2004)

### DISCLOSURE OF THE INVENTION

### Problem to be Solved by the Invention

An object of the present invention is to provide a compound having an excellent cysteine protease inhibitory effect.

Another object of the present invention is to provide a compound useful for the treatment or prevention of a disease selected from the group consisting of osteoporosis, osteoarthritis, chronic rheumatoid arthritis, Paget's disease of bone, bone metastasis of cancer, and ostealgia.

### Means to Solve the Problem

As a result of extensive study regarding the compounds having a cysteine protease inhibitory effect, the present inventors found that compounds and the salts thereof having a structure in which a methylene substituted with a trifluoromethyl is introduced as characteristics of chemical structure, such as the compounds represented by the following formula (1):

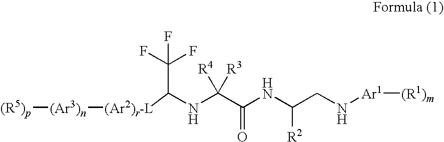

have an especially good cysteine protease inhibitory effect, and completed the present invention based on these findings.

That is, the present invention relates to the followings.

(1) A compound represented by formula (1), or a pharmaceutically acceptable salt thereof

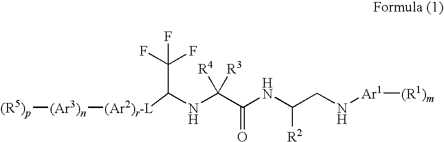

(In formula (1),
Ar¹ represents C₆-C₁₀ aryl, or heteroaryl;
R¹ represents a substituent selected from the substituent group 1;
m represents an integer of 0 to 3;
R² represents C₁-C₆ alkyl that may be substituted with the same or different 1 to 6 group(s) selected from the substituent group 2;
R³ and R⁴ are the same or different from each other and represent hydrogen atom or C₁-C₆ alkyl, C₃-C₇ cycloalkyl, C₄-C₉ (cycloalkyl)alkyl, phenyl, heteroaryl, C₇-C₉ phenylalkyl, or C₁-C₃ alkyl substituted with heteroaryl, these substituents may be substituted with the same or different 1 to 6 group(s) selected from the substituent group 3;
when both of R³ and R⁴ are C₁-C₆ alkyl that may be substituted with the same or different 1 to 6 group(s) selected from the substituent group 3, the R³ and R⁴ may bond each other via a single bond, —O—, —NR⁹—, or —S(O)_{q}— to form 3- to 7-membered ring structure containing the carbon atoms to which R³ and R⁴ are bonding;
when R³ and R⁴ do not bond to form a ring structure, either R³ or R⁴ represents a group which is not a hydrogen atom;
L represents a single bond or —(CR¹⁰R¹¹)ₛ—;
s represents any one integer of 1 to 4;
Ar² represents C₆-C₁₀ aryl or heteroaryl;
r represents 0 or 1;
Ar³ represents C₆-C₁₀ aryl or heteroaryl;
n represents 0 or 1;
R⁵ represents a substituent selected from the substituent group 1;
p represents an integer of 0 to 5;
the substituent group 1 represents a group consisting of hydrogen atom, halogen atom, cyano, nitro, R^{6a}, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)₂N(R^{6a})(R^{6b}), —NR^{6a}S(O)₂R^{6b}, —S(O)_{q}R^{6a}, and —Si(R⁸)₃;
the substituent group 2 represents a group consisting of halogen atom, cyano, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)_{q}R^{6a}, —N(R^{6a})C(═NR^{6b})(NR^{6c}), C₃-C₇ cycloalkyl that may be substituted with R⁷, phenyl that may be substituted with R⁷, and heteroaryl that may be substituted with R⁷;
the substituent group 3 represents halogen atom, hydroxyl, and C₁-C₆ alkoxy, C₁-C₆ alkylthio, C₁-C₆ alkylsulfinyl, or C₁-C₆ alkylsulfonyl, these substituents may be substituted with halogen atom;
R^{6a}, R^{6b}, and R^{6c} are the same or different from each other and represent hydrogen atom, C₁-C₆ alkyl that may be substituted with R⁷, C₂-C₆ alkenyl that may be substituted with R⁷, C₂-C₆ alkynyl that may be substituted with R⁷, C₃-C₇ cycloalkyl that may be substituted with R⁷, heterocyclyl that may be substituted with R⁷, phenyl that may be substituted with R⁷, heteroaryl that may be substituted with R⁷, C₇-C₁₃ aralkyl that may be substituted with R⁷, C₁-C₃ alkyl substituted with heterocyclyl that may be substituted with R⁷, or C₁-C₃ alkyl substituted with heteroaryl that may be substituted with R⁷; in each substituent in the substituent groups 1 and 2, the R^{6a} and R^{6b}, R^{6a} and R^{6c}, or R^{6b} and R^{6c} may bond each other via a single bond, —O—, —NR⁹—, or —S(O)_{q}— to form 3- to 7-membered ring structure, when R^{6a} and R^{6b}, R^{6a} and R^{6c}, R^{6b} and R^{6c} existing in one substituent are C₁-C₆ alkyl optionally substituted with R⁷;
q represents an integer of 0 to 2;
R⁷ represents halogen atom, hydroxyl, carboxyl, C₁-C₄ alkyl, C₁-C₄ alkoxy, C₁-C₄ alkoxycarbonyl, C₁-C₄ alkylsulfonyl, C₁-C₄ alkylsulfinyl, or cyano;
R⁸ represents C₁-C₆ alkyl that may be substituted with R⁷; and
R⁹, R¹⁰, and R¹¹ are the same or different from each other and represent hydrogen atom or C₁-C₆ alkyl that may be substituted with R⁷.)
(2) The compound described in (1) and represented by formula (1A), or a pharmaceutically acceptable salt thereof.

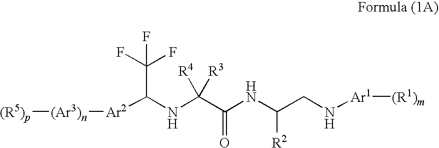

(In formula (1A),
Ar¹ represents C₆-C₁₀ aryl, or heteroaryl;
R¹ represents a substituent selected from the substituent group 1;
m represents an integer of 0 to 3;
R² represents C₁-C₆ alkyl that may be substituted with the same or different 1 to 6 group(s) selected from the substituent group 2;
R³ and R⁴ are the same or different from each other and represent hydrogen atom or C₁-C₆ alkyl, C₃-C₇ cycloalkyl, C₄-C₉ (cycloalkyl)alkyl, phenyl, heteroaryl, C₇-C₉ phenylalkyl, or C₁-C₃ alkyl substituted with heteroaryl, these substituents may be substituted with the same or different 1 to 6 group(s) selected from the substituent group 3; when both of R³ and R⁴ are C₁-C₆ alkyl that may be substituted with the same or different 1 to 6 group(s) selected from the substituent group 3, the R³ and R⁴ may bond each other via a single bond, —O—, —NR⁹—, or —S(O)_{q}— to form 3- to 7-membered ring structure containing the carbon atoms to which R³ and R⁴ are bonding;
when R³ and R⁴ do not bond to form a ring structure, either R³ or R⁴ represents a group which is not a hydrogen atom;
Ar² represents C₆-C₁₀ aryl or heteroaryl;
Ar³ represents C₆-C₁₀ aryl or heteroaryl;
n represents 0 or 1;
R⁵ represents a substituent selected from the substituent group 1;
p represents an integer of 0 to 5;
the substituent group 1 represents a group consisting of halogen atom, cyano, nitro, —R^{6a}, —OR^{6a}, O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)₂N(R^{6a})(R^{6b}), —NR^{6a}S(O)₂R^{6b}, —S(O)_{q}R^{6a}, and —Si(R⁸)₃;
the substituent group 2 represents a group consisting of halogen atom, cyano, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)_{q}R^{6a}, C₃-C₇ cycloalkyl that may be substituted with R⁷, phenyl that may be substituted with R⁷, and heteroaryl that may be substituted with R⁷;
the substituent group 3 represents halogen atom, hydroxyl, and C₁-C₆ alkoxy, C₁-C₆ alkylthio, C₁-C₆ alkylsulfinyl, and C₁-C₆ alkylsulfonyl, these substituents may be substituted with a halogen atom;
R^{6a}, R^{6b}, and R^{6c} are the same or different from each other and represent hydrogen atom, C₁-C₆ alkyl that may be substituted with R⁷, C₂-C₆ alkenyl that may be substituted with R⁷, C₂-C₆ alkynyl that may be substituted with R⁷, C₃-C₇ cycloalkyl that may be substituted with R⁷, heterocyclyl that may be substituted with R⁷, phenyl that may be substituted with R⁷, heteroaryl that may be substituted with R⁷, C₇C₁₃ aralkyl that may be substituted with R⁷, C₁-C₃ alkyl substituted with heterocyclyl that may be substituted with R⁷, or C₁-C₃ alkyl substituted with heteroaryl that may be substituted with R⁷; in each substituent in the substituent groups 1 and 2, the R^{6a} and R^{6b}, R^{6a} and R^{6c}, or R^{6b} and R^{6c} may bond each other via a single bond, —O—, —NR⁹—, or —S(O)_{q}— to form 3- to 7-membered ring structure, when R^{6a} and R^{6b}, R^{6a} and R^{6c} or R^{6b} and R^{6c} existing in one substituent are C₁-C₆ alkyl optionally substituted with R⁷;
q represents an integer of 0 to 2;
R⁷ represents halogen atom, hydroxyl, carboxyl, C₁-C₄ alkyl, C₁-C₄ alkoxy, C₁-C₄ alkoxycarbonyl, C₁-C₄ alkylsulfonyl, or C₁-C₄ alkylsulfinyl; and
R⁸ and R⁹ are the same or different from each other and represent C₁-C₆ alkyl that may be substituted with R⁷.)
(3) The compound described in (1) or (2), or a pharmaceutically acceptable salt thereof,
wherein R³ represents C₁-C₆ alkyl, C₃-C₇ cycloalkyl, or C₄-C₉ (cycloalkyl)alkyl, these substituents may be substituted with 1 to 6 fluorine atom(s); and
R⁴ represents hydrogen atom.
(4) The compound described in (1) or (2), or a pharmaceutically acceptable salt thereof,
wherein R³ represents isobutyl that may be substituted with 1 to 6 fluorine atom(s); and
R⁴ represents hydrogen atom.
(5) The compound described in (1) or (2), or a pharmaceutically acceptable salt thereof,
wherein R³ and R⁴ form cyclohexane ring containing the carbon atoms to which R³ and
R⁴ are bonding.
(6) The compound described in any of (1) to (5), or a pharmaceutically acceptable salt thereof,
wherein Ar¹ represents C₆-C₁₀ aryl.
(7) The compound described in any of (1) to (6), or a pharmaceutically acceptable salt thereof,
in which m represents an integer of 1 to 3.
(8) The compound described in (7), or a pharmaceutically acceptable salt thereof,
wherein at least one R¹ represents —OR^{6a} or —N(R^{6a})(R^{6b}).
(9) The compound described in any of (1) to (5), or a pharmaceutically acceptable salt thereof,
wherein —Ar¹—(R¹)ₘ is a substituent represented by formula (2).

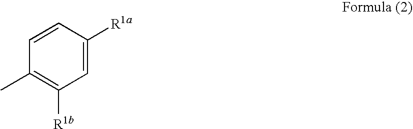

(In formula (2), R^{1a} represents —OR^{6a} or —N(R^{6a})(R^{6b}); and
R^{1b} represents halogen atom, —R^{6a}, —OR^{6a}, or —N(R^{6a})(R^{6b}).)
(10) The compound described in any of (1) to (5), or a pharmaceutically acceptable salt thereof,
wherein —Ar¹—(R¹)ₘ is a substituent represented by formula (3).

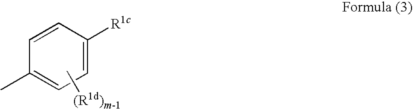

(In formula (3), R^{1c} represents —N(R^{6a})(R^{6b}); and
R^{1d} represents a substituent selected from the substituent group 1.)
(11) The compound described in any of (1) to (10), or a pharmaceutically acceptable salt thereof,
wherein at least one of R¹, the substituent of R¹, the substituent of R² selected from the substituent group 2, R⁵, and the substituent of R⁵ represents —COOH.
(12) The compound described in any of (1) to (10), or a pharmaceutically acceptable salt thereof,
wherein the substituent of R² selected from the substituent group 2 represents —N(R^{6a})(R^{6b}) or —N(R^{6a})C(═NR^{6b})(NR^{6c}).
(13) The compound described in any of (1) to (10), or a pharmaceutically acceptable salt thereof,
wherein at least one of R¹, the substituent of R¹, the substituent of R² selected from the substituent group 2, R⁵, and the substituent of R⁵ represents cyano.
(14) The compound described in any of (1) to (5), or a pharmaceutically acceptable salt thereof,
wherein Ar¹ represents heteroaryl.
(15) The compound described in any of (1) to (14), or a pharmaceutically acceptable salt thereof,
wherein Ar² represents C₆-C₁₀ aryl.
(16) The compound described in any of (1) to (14), or a pharmaceutically acceptable salt thereof,
wherein Ar² represents heteroaryl.
(17) A pharmaceutical composition comprising the compound described in any of (1) to (16), or a pharmaceutically acceptable salt thereof and a pharmaceutically acceptable carrier.
(18) A cathepsin K inhibitor comprising the compound described in any of (1) to (16), or a pharmaceutically acceptable salt thereof as an active ingredient.
(19) A drug comprising the compound described in any of (1) to (16), or a pharmaceutically acceptable salt thereof as an active ingredient for treatment or prevention of a disease selected from the group consisting of osteoporosis, osteoarthritis, chronic rheumatoid arthritis, Paget's disease of bone, hypercalcemia, bone metastasis of cancer, and ostealgia.

The present invention provides a novel compound having an excellent cysteine protease inhibitory effect (especially a cathepsin K inhibitory effect).

Furthermore, the present invention provides a drug for treatment or prevention of a disease selected from a group consisting of osteoporosis, osteoarthritis, chronic rheumatoid arthritis, Paget's disease of bone, hypercalcemia, bone metastasis of cancer, and ostealgia.

### MODE FOR CARRYING OUT THE INVENTION

Terms used alone or in combination in this specification will be explained below. Unless otherwise mentioned particularly, explanation of each substituent shall be common at each position. Note that when each of any variables (for example, R^{6a}, R^{6b}, R^{6c}, R⁷, R⁸, R⁹, and the like) exists in any component (R¹, R², R⁵, and the like), its definition is independent in each component. In addition, combination of substituents and variables are allowed only when such combination results in a chemically stable compound. When a substituent itself is substituted with two or more groups, these plural groups can be present on the same or different carbon as far as a stable structure forms.

In the present invention, “C₆-C₁₀ aryl” means a group which forms by elimination of one hydrogen atom bonding to a ring of an aromatic hydrocarbon having 6 to 10 carbon atoms. Examples include, but are not limited to, phenyl, naphthyl, indenyl, tetrahydronaphthyl, indanyl, and azulenyl.

In the present invention, “C₇-C₁₃ aralkyl” means a group which forms by substitution in alkyl having 1 to 3 carbon atom(s) with the above-mentioned one C₆-C₁₀ aryl at any position. Examples include, but are not limited to, benzyl, phenethyl, naphthylmethyl, and naphthylethyl.

In the present invention, “heteroaryl” means 3- to 10-membered monocyclic or bicyclic heterocylic system having an aromaticity, containing 1 to 5 hetero atom(s) selected from a group consisting of oxygen, sulfur, and nitrogen. “3- to 10-membered monocyclic or bicyclic heterocyclic system having an aromaticity” means a monovalent group obtained by eliminating a hydrogen atom from 3- to 10-membered monocyclic or bicyclic aromatic hetero ring having 1 to 5 hetero atom(s) selected from a group consisting of oxygen, sulfur, and nitrogen. In addition, in the case of bicyclic heteroaryl, when one ring is an aromatic ring or an heteroaryl ring, the other ring may have a non-aromatic ring structure. Number of each hetero atom and their combination in such heteroaryl is not particularly limited as far as the ring can be constituted with a predetermined number of the members and exists chemically stably. Examples of such heteroaryl include, but are not limited to, pyridyl, pyrazyl, pyrimidyl, pyridazinyl, furyl, thienyl, pyrazolyl, 1,3-dioxindanyl, isoxazolyl, isothiazolyl, benzofuranyl, isobenzofuryl, benzothienyl, indolyl, isoindolyl, chromanyl, benzothiazolyl, benzoimidazolyl, benzoxazolyl, pyranyl, imidazolyl, oxazolyl, thiazolyl, triazinyl, triazolyl, furazanyl, thiadiazolyl, dihydrobenzofuryl, dihydroisobenzofuryl, dihydroquinolyl, dihydroisoquinolyl, dihydrobenzoxazolyl, dihydropteridinyl, benzoxazolyl, benzisoxazolyl, benzodioxazolyl, quinolyl, isoquinolyl, benzotriazolyl, pteridinyl, purinyl, quinoxalinyl, quinazolinyl, cinnolinyl, or tetrazolyl.

In the present invention, “heterocyclyl” means a monovalent group obtained by eliminating a hydrogen atom from 3- to 10-membered monocyclic or bicyclic aliphatic hetero ring, which may be partially unsaturated or saturated, containing 1 to 4 hetero atom(s) selected from a group consisting of oxygen, sulfur, and nitrogen as a hetero atom. The heterocyclyl may contain 1 or 2 —C(═O)— or —C(═S)— in the ring. Number of each heteroatom and their combination is not particularly limited as far as the ring can be constituted with a predetermined number of the members and exists chemically stably. Examples of such heterocyclyl include, but are not limited to, piperidyl, piperidino, pyrrolidinyl, pyrrolinyl, tetrahydrofuryl, dihydropyranyl, hexahydroazepinyl, piperazinyl, quinuclidinyl, morpholinyl, morpholino, thiomorpholinyl, thiomorpholino, oxazolinyl, 1,4-dioxanyl, pyranyl, 2-pyrrolidonyl, 2-piperidonyl, 2-imidazolidinonyl, or tetrahydro-3H-pyrazol-3-onyl.

In the present invention, “halogen atom” means fluorine, chlorine, bromine, and iodine.

In the present invention, “C₁-C₆ alkyl” means a saturated linear or branched chain aliphatic hydrocarbon group having 1 to 6 carbon atom(s). Examples include, but are not limited to, methyl, ethyl, n-propyl, n-butyl, n-pentyl, n-hexyl, isopropyl, isobutyl, s-butyl, t-butyl, isopentyl, 2-methylbutyl, neopentyl, 1-ethylpropyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 1-ethylbutyl, 2-ethylbutyl, t-pentyl, and isohexyl.

In the present invention, “C₃-C₇ cycloalkyl” means a cycloalkyl group having 3 to 7 carbon atoms. Examples include, but are not limited to, a cyclic alkyl group such as cyclopropyl, cyclobutyl, cyclopentyl, cyclohexyl, and cycloheptyl groups.

In the present invention, “C₄-C₉ (cycloalkyl)alkyl” means a group formed by substitution in the above-mentioned “C₁-C₃ alkyl” with the above-mentioned one “C₃-C₇ cycloalkyl” at any position. Examples include, but are not limited to, cyclopropylmethyl, cyclobutylmethyl, cyclopentylmethyl, cyclohexylmethyl, cycloheptylmethyl, cyclopropylethyl, cyclobutylethyl, cyclopentylethyl, cyclohexylethyl, and cycloheptylethyl.

In the present invention, “C₇-C₉ phenylalkyl” means a group formed by substitution in the above-mentioned “C₁-C₃ alkyl” with one phenyl group at any position. Examples include, but are not limited to, benzyl, phenethyl, and phenylpropyl.

In the present invention, “C₁-C₆ alkoxy” means a group consisting of the above-mentioned “C₁-C₆ alkyl” and an oxy group. Examples include, but are not limited to, methoxy, ethoxy, n-propoxy, isopropoxy, n-butoxy, s-butoxy, 2-methylpropoxy, n-pentyloxy, isopentyloxy, 2-methylbutoxy, 1-ethylpropoxy, 2,2-dimethylpropoxy, n-hexyloxy, 4-methylpentoxy, 3-methylpentoxy, 2-methylpentoxy, 3,3-dimethylbutoxy, 2,2-dimethylbutoxy, 1,1-dimethylbutoxy, and t-butoxy.

In the present invention, “C₁-C₆ alkylthio” means a group consisting of the above-mentioned “C₁-C₆ alkyl” and a thio group. Examples include, but are not limited to, methylthio, ethylthio, and isopropylthio.

In the present invention, “C₁-C₆ alkylsulfinyl” means a group consisting of the above-mentioned “C₁-C₆ alkyl” and a sulfinyl. Examples include, but are not limited to, methylsulfinyl, ethylsulfinyl, and isopropylsulfinyl.

In the present invention, “C₁-C₆ alkylsulfonyl” means a group consisting of the above-mentioned “C₁-C₆ alkyl” and a sulfonyl. Examples include, but are not limited to, methylsulfonyl, ethylsulfonyl, and isopropylsulfonyl.

In the present invention, “C₁-C₆ alkoxycarbonyl” means a group consisting of the above-mentioned “C₁-C₆ alkoxy” and a carbonyl. Examples include, but are not limited to, methoxycarbonyl, ethoxycarbonyl, and isopropoxycarbonyl.

In the present invention, “C₂ to C₆ alkenyl” means a linear or branched chain aliphatic hydrocarbon group having a double bond and 2 to 6 carbon atoms. Examples include, but are not limited to, vinyl, allyl, 1-propenyl, 2-butenyl, 3-butenyl, 2-methyl-1-propenyl, 2-methyl-2-propenyl, 4-pentenyl, 5-hexenyl, and 4-methyl-3-pentenyl.

In the present invention, “C₂ to C₆ alkynyl” means a linear or branched chain aliphatic hydrocarbon group having a triple bond and 2 to 6 carbon atoms. Examples include, but are not limited to, ethynyl, propargyl, 3-methylpropargyl, butynyl, 2-butyn-1-yl, pentynyl, and hexynyl.

In the present invention, “C₁-C₆ alkyl that may be substituted with the same or different 1 to 6 group(s) selected from the substituent group 2” means that the “C₁-C₆ alkyl” may be substituted with “the same or different 1 to 6 group(s) selected from the substituent group 2” at any position and that, when the “C₁-C₆ alkyl” is substituted with 2 to 6 groups selected from the substituent group 2, the “C₁-C₆ alkyl” may be substituted with the same group or a different group. Furthermore, “C₁-C₆ alkyl that may be substituted with the same or different 1 to 6 group(s) selected from the substituent group 3”, and the like, have the similar meaning.

In the group substituted with R⁷, such as “C₁-C₆ alkyl that may be substituted with R⁷”, “C₃-C₇ cycloalkyl that may be substituted with R⁷”, and the like, in the present invention, the upper limit of the substitution number of the substituent R⁷ is 10 when R⁷ is a halogen atom and 5 when R⁷ is the substituent other than a halogen atom. Among these, substitution number of R⁷ is preferably 0 to 3.

In addition, in the above-mentioned definition, for example, “C” in “C₁” or the like represents a carbon atom and the subsequent number represents the number of carbon atoms. For example, “C₁-C₆” represents a range from 1 carbon atom to 6 carbon atoms. It is naturally meant that, when the number of carbon atoms is different, the group has the different number of carbon atoms in the present invention. For example, “C₁-C₄ alkyl” means that the alkyl defined by “C₁-C₆ alkyl” has the number of carbon atoms of 1 to 4. The number of carbon atoms in other groups is the same as in the above.

The present invention relates to the compound represented by the above-mentioned formula (1) or the pharmaceutically acceptable salt thereof. Among these, the compound represented by the above-mentioned formula (1A) or the pharmaceutically acceptable salt thereof is preferable. Hereinafter, the definitions common in the compound represented by formula (1) and the compound represented by formula (1A) will be explained together.

In the above-mentioned formula (1) and formula (1A), Ar¹ represents C₆-C₁₀ aryl or heteroaryl. Specific examples of “aryl” and “heteroaryl” are as defined above. Examples of the preferred “aryl” or “heteroaryl” in Ar¹ include phenyl, pyrazolyl, benzofuranyl, benzothienyl, indolyl, benzothiazolyl, benzoimidazolyl, benzoxazolyl, thiazolyl, dihydrobenzofuranyl, dihydroisobenzofuranyl, dihydroquinolyl, dihydroisoquinolyl, dihydrobenzoxazolyl, dihydropteridinyl, benzoxazolyl, benzisoxazolyl, benzodioxazolyl, quinolyl, isoquinolyl, benzotriazolyl, quinoxalinyl, and quinazolinyl. Especially phenyl is preferred.

In the above-mentioned formula (1), R¹ represents a group selected from the substituent group 1. “Substituent group 1” represents a group consisting of hydrogen atom, halogen atom, cyano, nitro, —R^{6a}, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)₂N(R^{6a})(R^{6b}), —NR^{6a}S(O)₂R^{6b}, —S(O)_{q}R^{6a}, and —Si(R⁸)₃, wherein q represents an integer of 0 to 2.

In addition, R^{6a}, R^{6b}, and R^{6c} are the same or different from each other and represent hydrogen atom, C₁-C₆ alkyl that may be substituted with R⁷, C₂-C₆ alkenyl that may be substituted with R⁷, C₂-C₆ alkynyl that may be substituted with R⁷, C₃-C₇ cycloalkyl that may be substituted with R⁷, heterocyclyl that may be substituted with R⁷, phenyl that may be substituted with R⁷, heteroaryl that may be substituted with R⁷, C₇-C₁₃ aralkyl that may be substituted with R⁷, C₁-C₃ alkyl substituted with heterocyclyl that may be substituted with R⁷, or C₁-C₃ alkyl substituted with heteroaryl that may be substituted with R⁷. R⁸ represents C₁-C₆ alkyl that may be substituted with R⁷.

Furthermore, R⁷ represents halogen atom, hydroxyl, carboxyl, C₁-C₄ alkyl, C₁-C₄ alkoxy, C₁-C₄ alkoxycarbonyl, C₁-C₄ alkylsulfonyl, C₁-C₄ alkylsulfinyl, or cyano.

In addition, in each substituent in the substituent group 1, when R^{6a} and R^{6b}, R^{6a} and R^{6c}, or R^{6b} and R^{6c} present in one group are C₁-C₆ alkyls that may be substituted with R⁷, the R^{6a} and R^{6b}, R^{6a} and R^{6c}, or R^{6b} and R^{6c} may bond each other via a single bond, —O—, —NR⁹—, or —S(O)_{q}— to form 3- to 7-membered ring structure, wherein q represents an integer of 0 to 2 and R⁹ represents a hydrogen atom or C₁-C₆ alkyl that may be substituted with R⁷.

“3- to 7-membered ring structure” as R¹ may contain two or less heteroatoms selected from a group consisting of oxygen, nitrogen, and sulfur, as an atom forming such ring structure. Examples of R¹ which forms such “3- to 7-membered ring structure” include, but are not limited to, 1-piperidyl, 1-pyrrolidinyl, morpholino, thiomorpholino, 1,1-dioxothiomorpholin-4-yl, and 1-piperazinyl.

In the above-mentioned formula (1A), R¹ represents a group selected from the substituent group 1. “Substituent group 1” represents a group consisting of halogen atom, cyano, nitro, —R^{6a}, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)₂N(R^{6a})(R^{6b}), —NR^{6a}S(O)₂R^{6b}, —S(O)_{q}R^{6a}, and —Si(R⁸)₃, wherein q represents an integer of 0 to 2.

In addition, R^{6a}, R^{6b}, and R^{6c} are the same or different from each other and represent hydrogen atom, C₁-C₆ alkyl that may be substituted with R⁷, C₂-C₆ alkenyl that may be substituted with R⁷, C₂-C₆ alkynyl that may be substituted with R⁷, C₃-C₇ cycloalkyl that may be substituted with R⁷, heterocyclyl that may be substituted with R⁷, phenyl that may be substituted with R⁷, heteroaryl that may be substituted with R⁷, C₇-C₁₃ aralkyl that may be substituted with R⁷, C₁-C₃ alkyl substituted with heterocyclyl that may be substituted with R⁷, or C₁-C₃ alkyl substituted with heteroaryl that may be substituted with R⁷. R⁸ represents C₁-C₆ alkyl that may be substituted with R⁷.

Furthermore, R⁷ represents halogen atom, hydroxyl, carboxyl, C₁-C₄ alkyl, C₁-C₄ alkoxy, C₁-C₄ alkoxycarbonyl, C₁-C₄ alkylsulfonyl, or C₁-C₄ alkylsulfinyl.

In addition, in each substituent in the substituent group 1, when R^{6a} and R^{6b}, R^{6a} and R^{6c}, or R^{6b} and R^{6c} present in one group are C₁-C₆ alkyls that may be substituted with R⁷, the R^{6a} and R^{6b}, R^{6a} and R^{6c}, or R^{6b} and R^{6c} may bond each other via a single bond, —O—, —NR⁹—, or —S(O)_{q}— to form 3- to 7-membered ring structure, wherein q represents an integer of 0 to 2 and R⁹ represents C₁-C₆ alkyl that may be substituted with R⁷.

“3- to 7-membered ring structure” as R¹ may contain two or less heteroatoms selected from a group consisting of oxygen, nitrogen, and sulfur, as an atom forming such ring structure. Examples of R¹ which forms such “3- to 7-membered ring structure” include, but are not limited to, 1-piperidyl, 1-pyrrolidinyl, morpholino, thiomorpholino, 1,1-dioxothiomorpholin-4-yl, and 1-piperazinyl.

In the above-mentioned formula (1) and formula (1A), examples of especially preferred R¹ are halogen atom, —R^{6a}, —OR^{6a}, and —N(R^{6a})(R^{6b}).

In the above-mentioned formula (1), m represents an integer of 0 to 3, preferably an integer of 1 to 3.

In addition, examples of preferred combination of “Ar¹”, “R¹”, and “m” (—Ar¹—(R¹)ₘ) may be represented by the following structural formulae.

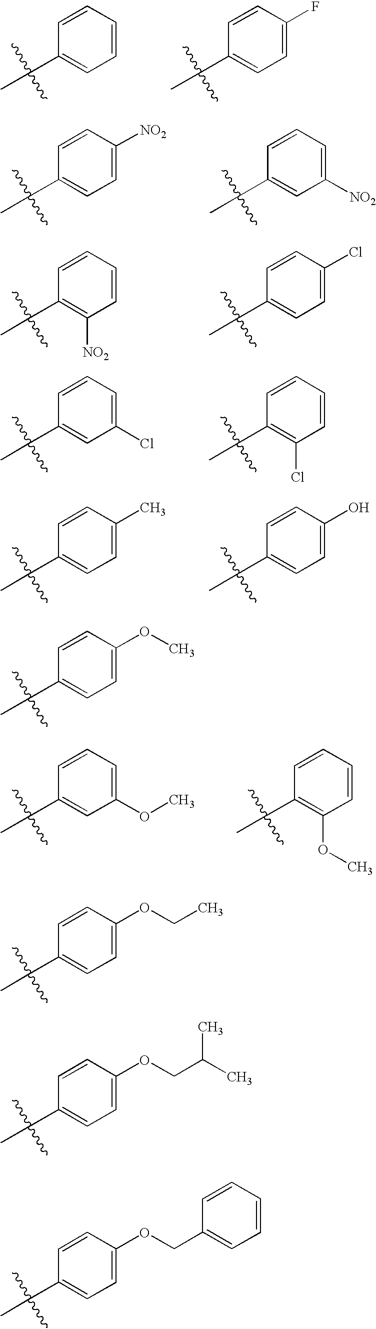

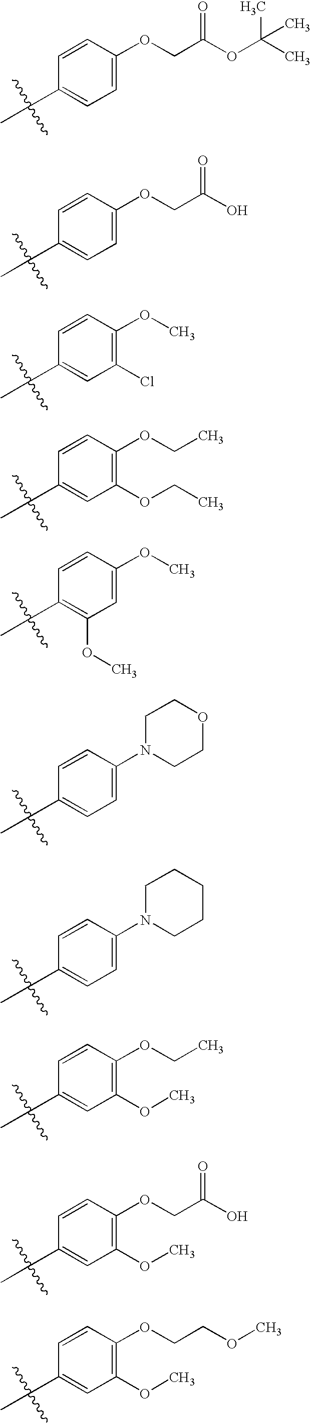

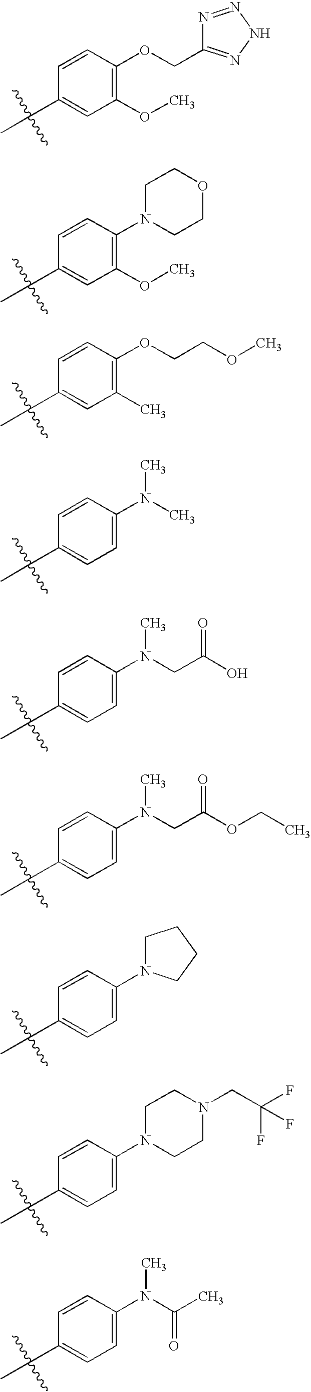

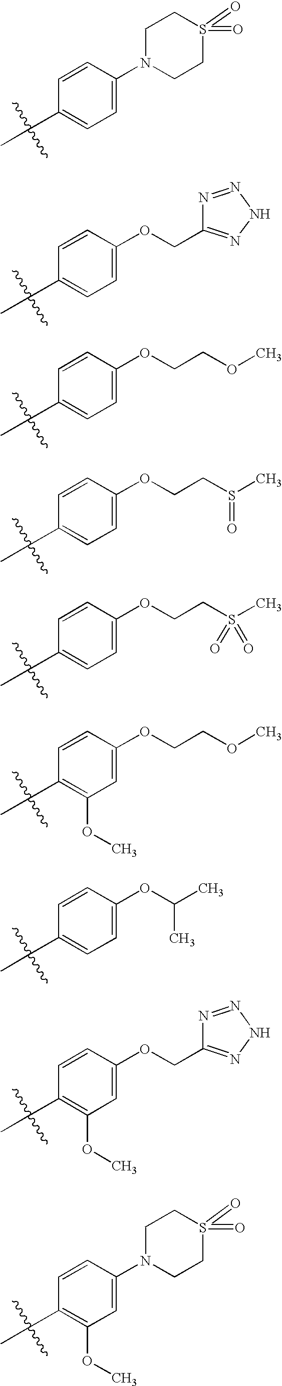

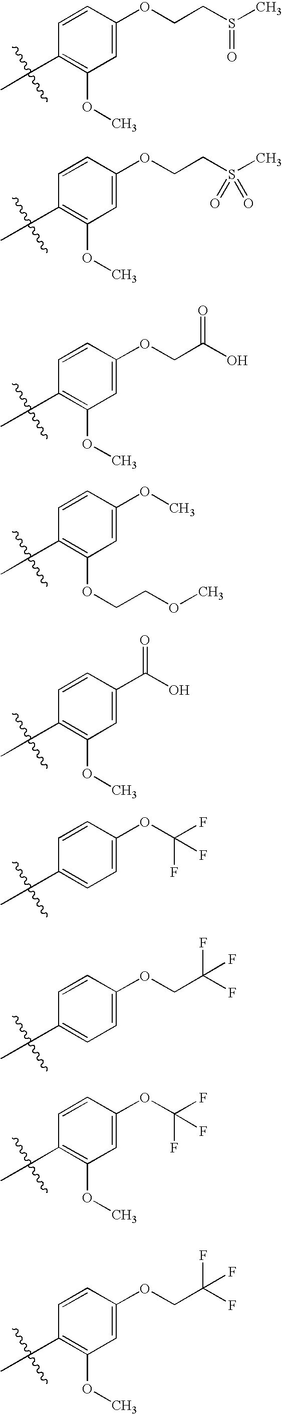

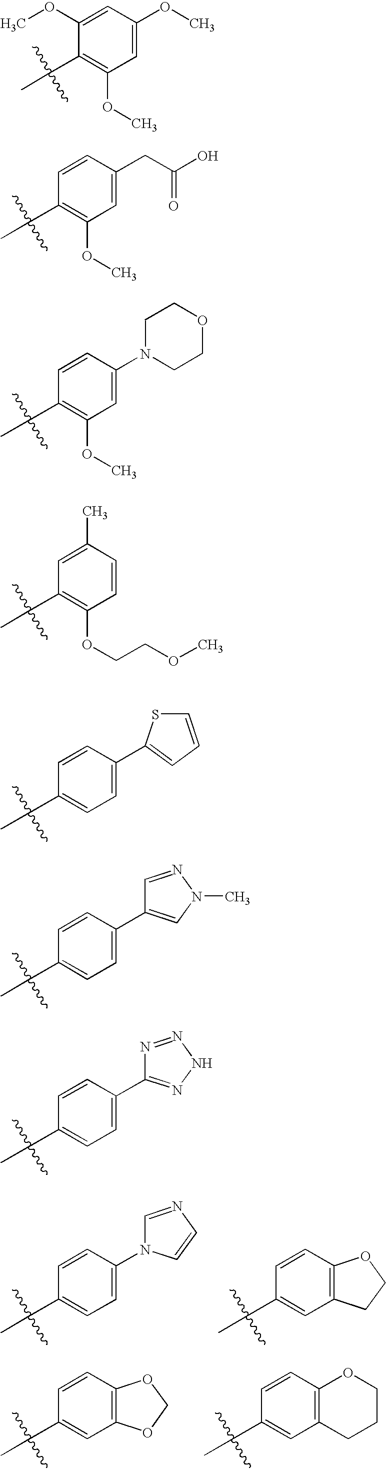

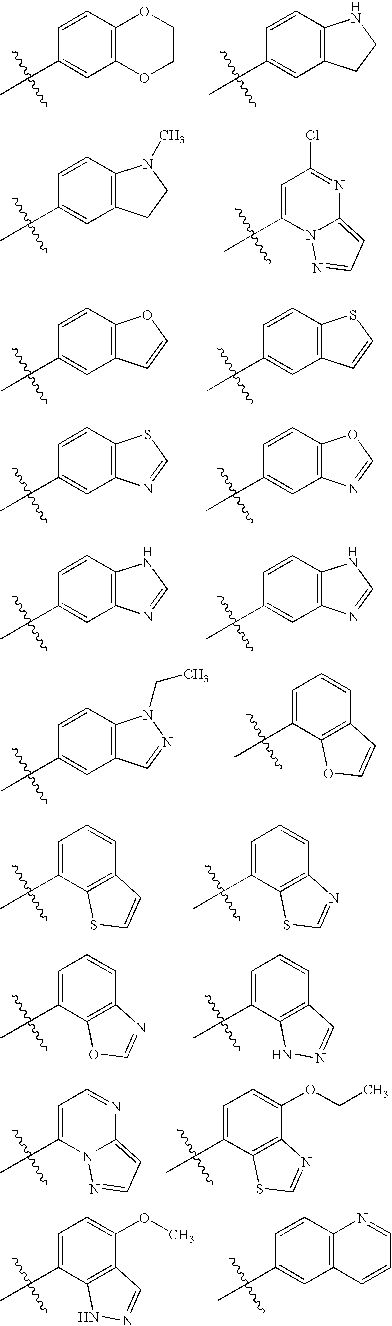

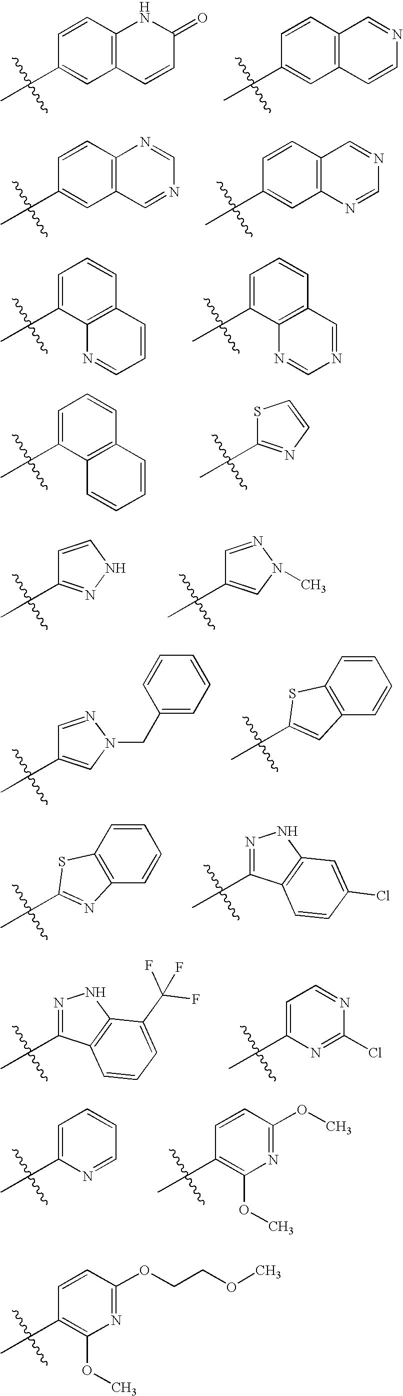

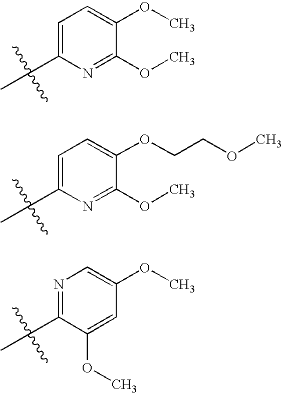

An example of especially more preferred combination of “Ar¹”, “R¹”, and “m” (—Ar¹—(R¹)ₘ) is a substituent represented by the following formula (2):

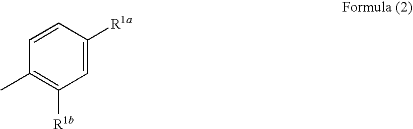

(In formula (2), R^{1a} represents —OR^{6a} or —N(R^{6a})(R^{6b}); and R^{1b} represents a halogen atom, —R^{6a}, —OR^{6a}, or —N(R^{6a})(R^{6b})).

In addition, definition of R^{6a} and R^{6b} in R^{1a} and R^{1b} is the same as the definition of R^{6a} and R^{6b} in the above-mentioned R¹.

In formula (2), especially preferred R^{1a} is exemplified by —N(R^{6a})(R^{6b}).

Another example of especially more preferred combination of “Ar¹”, “R¹”, and “m” (—Ar¹—(R¹)ₘ) is a substituent represented by the following formula (3):

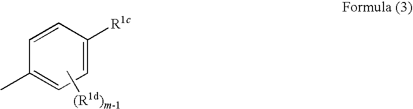

(In formula (3), R^{1c} represents —N(R^{6a})(R^{6b}); and R^{1d} represents a group selected from the substituent group 1).

In addition, definition of R^{6a} and R^{6b} in R^{1c} is the same as the definition of R^{6a} and R^{6b} in R¹ in the above-mentioned formula (1A). Definition of the substituent selected from the substituent group 1 in R^{1d} is the same as the definition of the substituent selected from the substituent group 1 in the above-mentioned formula (1A).

In addition, in formula (2) and (3), when R^{1a}, R^{1b}, R^{1c} and R^{1d} represent —N(R^{6a})(R^{6b}) and such R^{6a} and R^{6b} each represent the C₁-C₆ alkyl that may be substituted with R⁷, such R^{6a} and R^{6b} may form the above-mentioned “3- to 7-membered ring structure”.

In the above-mentioned formula (1), R² represents C₁-C₆ alkyl that may be substituted with the same or different 1 to 6 group(s) selected from the substituent group 2. “Substituent group 2” represents a group consisting of halogen atom, cyano, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)_{q}R^{6a}, —N(R^{6a})C(═NR^{6b})(NR^{6c}), C₃-C₇ cycloalkyl that may be substituted with R⁷, phenyl that may be substituted with R⁷, and heteroaryl that may be substituted with R⁷.

In addition, definition of “R^{6a}”, “R^{6b}”, “R^{6c}”, and “R⁷” in “substituent group 2” is the same as the definition of “R^{6a}”, “R^{6b}”, “R^{6c}”, and “R⁷” in “substituent group 1” in the above-mentioned formula (1).

In addition, in each substituent in the substituent group 2, when R^{6a} and R^{6b}, R^{6a} and R^{6c}, or R^{6b} and R^{6c} present in one group are C₁-C₆ alkyls that may be substituted with R⁷, the R^{6a} and R^{6b}, R^{6a} and R^{6c}, or R^{6b} and R^{6c} may bond each other via a single bond, —O—, —NR⁹—, or —S(O)_{q}— to form 3- to 7-membered ring structure, wherein R⁸ represents a C₁-C₆ alkyl that may be substituted with R⁷.

“3- to 7-membered ring structure” as R² may contain two or less heteroatoms selected from a group consisting of oxygen, nitrogen, and sulfur, as an atom forming such ring structure. Examples of the group selected from the substituent group 2 which forms such “3- to 7-membered ring structure” include, but are not limited to, 1-piperidyl, 1-pyrrolidinyl, morpholino, and 1-piperazinyl.

In the above-mentioned formula (1A), R² represents C₁-C₆ alkyl that may be substituted with the same or different 1 to 6 group(s) selected from the substituent group 2. “Substituent group 2” represents a group consisting of halogen atom, cyano, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)_{q}R^{6a}, C₃-C₇ cycloalkyl that may be substituted with R⁷, phenyl that may be substituted with R⁷, and heteroaryl that may be substituted with R⁷.

In addition, definition of “R^{6a}”, “R^{6b}”, “R^{6c}”, and “R⁷” in “substituent group 2” is the same as the definition of “R^{6a}”, “R^{6b}”, “R^{6c}”, and “R⁷” in “substituent group 1” of the above-mentioned formula (1A).

In addition, in each substituent in the substituent group 2, when R^{6a} and R^{6b}, R^{6a} and R^{6c} or R^{6b} and R^{6c} present in one group are C₁-C₆ alkyls that may be substituted with R⁷, the R^{6a} and R^{6b}, R^{6a} and R^{6c}, or R^{6b} and R^{6c} may bond each other via a single bond, —O—, —NR⁹—, or —S(O)_{q}— to form 3- to 7-membered ring structure, wherein R⁸ represents C₁-C₆ alkyl that may be substituted with R⁷.

“3- to 7-membered ring structure” as R² may contain two or less heteroatoms selected from a group consisting of oxygen, nitrogen, and sulfur, as an atom forming such ring structure. Examples of the group selected from the substituent group 2 which forms such “3- to 7-membered ring structure” include, but are not limited to, 1-piperidyl, 1-pyrrolidinyl, morpholino, and 1-piperazinyl.

In the above-mentioned formula (1) and formula (1A), specific examples of preferred R² include the substituents represented by the following formulae.

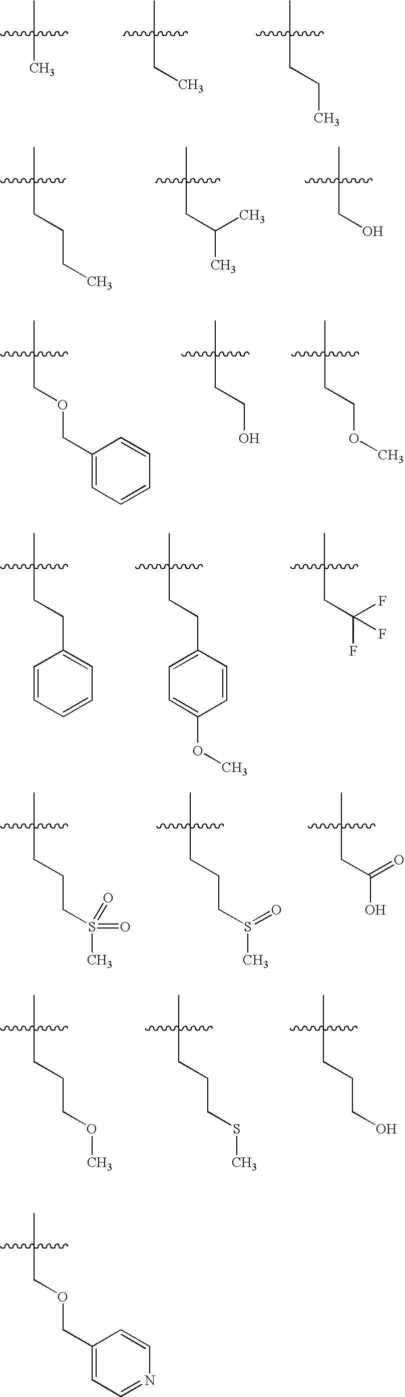

In the above-mentioned formula (1) and formula (1A), R³ and R⁴ are the same or different from each other and represent hydrogen atom or C₁-C₆ alkyl, C₃-C₇ cycloalkyl, C₄-C₉ (cycloalkyl)alkyl, phenyl, heteroaryl, C₇C₉ phenylalkyl, and C₁-C₃ alkyl substituted with heteroaryl, these groups may be substituted with the same or different 1 to 6 group(s) selected from the substituent group 3. “Substituent group 3” represents halogen atom, hydroxyl, and C₁-C₆ alkoxy, C₁-C₆ alkylthio, C₁-C₆ alkylsulfinyl, and C₁-C₆ alkylsulfonyl, these groups may be substituted with halogen atom. In addition, when both of R³ and R⁴ are C₁-C₆ alkyls that may be substituted with the same or different 1 to 6 group(s) selected from the substituent group 3, the R³ and R⁴ may bond each other via a single bond, —O—, —NR⁹—, or —S(O)_{q}— to form 3- to 7-membered ring structure containing the carbon atoms to which R³ and R⁴ are bonding, wherein q represents an integer of 0 to 2 and R⁹ represents C₁-C₆ alkyl that may be substituted with hydrogen atom or R⁷ in formula (1) and C₁-C₆ alkyl that may be substituted with R⁷ in formula (1A).

“3- to 7-membered ring structure” formed by R³ and R⁴ may contain two or less heteroatoms selected from a group consisting of oxygen, nitrogen, and sulfur, as an atom forming such ring structure. Examples of such “3- to 7-membered ring structure” include, but are not limited to, a ring structure such as cyclopropane, cyclobutane, cyclopentane, cyclohexane, cycloheptane, tetrahydrofuran, tetrahydropyran, pyrrolidine, piperidine, thiolane, and thiane.

In addition, when R³ and R⁴ do not bond to form a ring structure, either R³ or R⁴ represents a group which is not hydrogen atom.

Examples of preferred combination of R³ and R⁴ include the groups represented by the following formulae:

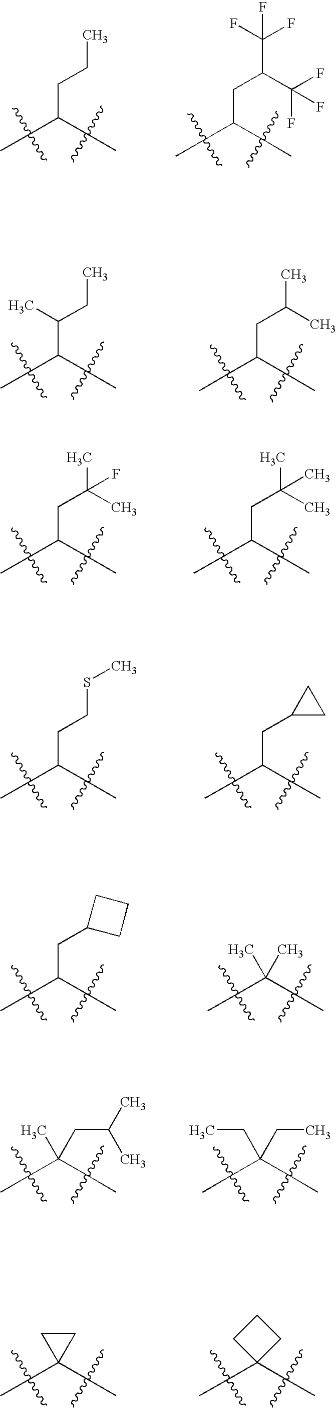

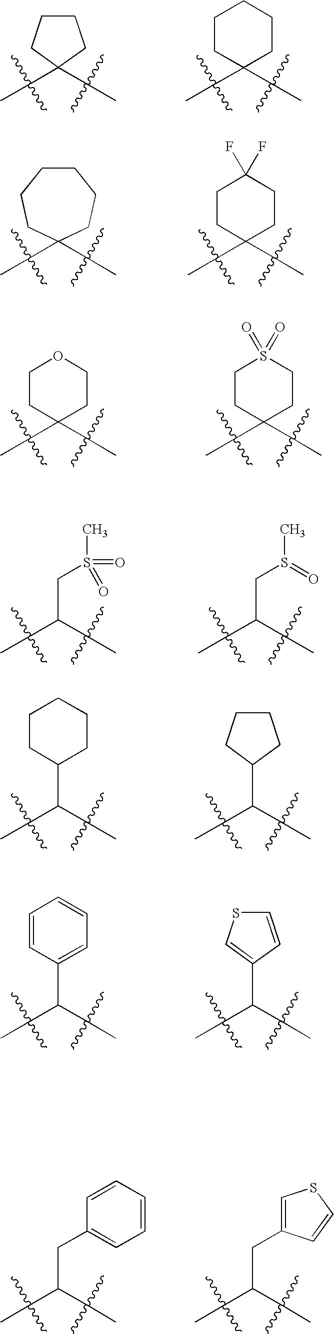

A specific example of more preferred combination of R³ and R⁴ is the combination in which R³ represents C₁-C₆ alkyl, C₃-C₇ cycloalkyl, or C₄-C₉ (cycloalkyl)alkyl, these groups may be substituted with 1 to 6 fluorine atom(s) and R⁴ represents a hydrogen atom. Especially preferable is the combination in which R³ represents isobutyl that may be substituted with 1 to 6 fluorine atom(s) and R⁴ represents hydrogen atom.

Another specific example of more preferred combination of R³ and R⁴ is the combination in which R³ and R⁴ form a cyclohexane ring containing the carbon atoms to which R³ and R⁴ are bonding.

In the above-mentioned formula (1), L represents a single bond or —(CR¹⁰R¹¹)ₛ—, wherein s represents any integer of 1 to 4. R¹⁰ and R¹¹ are the same or different from each other and represent hydrogen atom or C₁-C₆ alkyl that may be substituted with R⁷.

Among these, L is preferably a single bond.

In the above-mentioned formula (1) and formula (1A), Ar² represents C₆-C₁₀ aryl or heteroaryl. Specific examples of “aryl” and “heteroaryl” are the same as the above-mentioned definition. Examples of preferred “aryl” or “heteroaryl” of Ar² include phenyl, naphthyl, pyridyl, thienyl, pirazolyl, benzofuryl, benzothienyl, indolyl, benzothiazolyl, benzoimidazolyl, benzoxazolyl, imidazolyl, and thiazolyl. Among these, C₆-C₁₀ aryl (especially phenyl) or pyridyl is preferable. In addition, when Ar² represents “heteroaryl”, the metabolic stability is excellent. Among these, it is especially excellent when the heteroaryl ring represents a pyridine ring substituted with a hydroxyl, i.e., pyridone ring.

In the above-mentioned formula (1), r represents 0 or 1, preferably 1. When r represents 0, n which will be mentioned later represents 0.

In the above-mentioned formula (1) and formula (1A), Ar³ represents C₆-C₁₀ aryl or heteroaryl. Specific examples of “aryl” and “heteroaryl” are the same as the above-mentioned definition. Examples of preferred “aryl” or “heteroaryl” of Ar³ include phenyl, pyridyl, pyrazinyl, pyrimidinyl, pyridazinyl, furyl, thienyl, pyrazolyl, isoxazolyl, isothiazolyl, imidazolyl, and thiazolyl.

In the above-mentioned formula (1) and formula (1A), n represents 0 or 1.

When n represents 1, Ar² and Ar³ each is preferably monocyclic “aryl” and “heteroaryl”.

In the above-mentioned formula (1) and formula (1A), R⁵ represents a group selected from the substituent group 1. Definition of “substituent group 1”, “R^{6a}”, “R^{6b}”, “R^{6c}”, “R⁷”, and “q” in “R⁵” of the above-mentioned formula (1) and formula (1A) is the same as the definition of “substituent group 1”, “R^{6a}”, “R^{6b}”, “R^{6c}”, “R⁷”, and “q” in “R¹” of the above-mentioned formula (1) and formula (1A). Among these, specific examples of preferred R⁵ are halogen atom, cyano, —R^{6a}, —OR^{6a}, —COOR^{6a}, and —N(R^{6a})(R^{6b}).

“3- to 7-membered ring structure” as R⁵ may contain two or less heteroatoms selected from a group consisting of oxygen, nitrogen, and sulfur, as an atom forming such ring structure. Examples of R⁵ which forms such “3- to 7-membered ring structure” include, but are not limited to, 1-piperidyl, 1-pyrrolidinyl, morpholino, thiomorpholino, 1,1-dioxothiomorpholin-4-yl, and 1-piperazinyl.

In the above-mentioned formula (1) and formula (1A), p represents an integer of 0 to 5, preferably an integer of 0 to 3.

In the above-mentioned formula (1) and formula (1A), the compound or the pharmaceutically acceptable salt thereof of which at least one of R¹, the substituent of R¹, the substituent of R² selected from the substituent group 2, R⁵, and the substituent of R⁵ represents —COOH has an excellent metabolic stability and preferable. Similarly, in the above-mentioned formula (1) and formula (1A), the compound or the pharmaceutically acceptable salt thereof of which the group selected from the substituent group 2 substituting R² represents —N(R^{6a})(R^{6b}) or —N(R^{6a})C(═NR^{6b})(NR^{6c}), as well as the compound or the pharmaceutically acceptable salt thereof of which at least one of R¹, the substituent of R¹, the substituent of R² selected from the substituent group 2, R⁵, and the substituent of R⁵ represents cyano are excellent in metabolic stability and preferable.

In addition, examples of preferred combination of “L”, “Ar²”, “Ar³”, “R⁵”, “r”, “n”, and “p” ((R⁵)ₚ—(Ar³)ₙ—(Ar²)ᵣ-L-) may be represented by the following structural formulae.

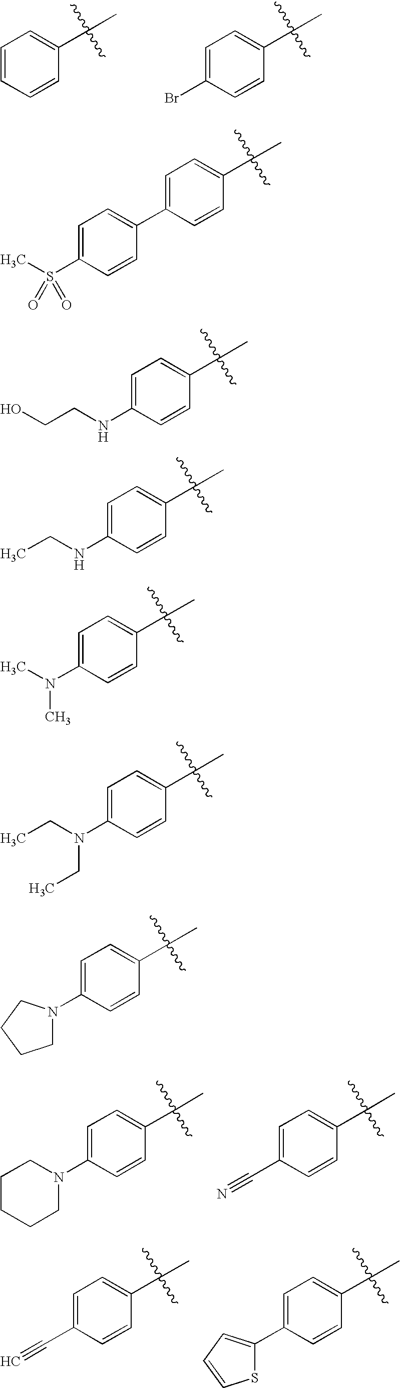

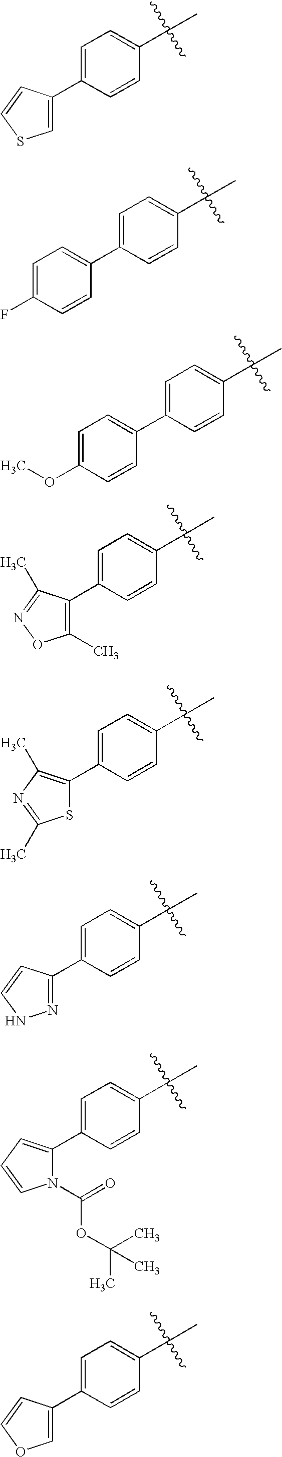

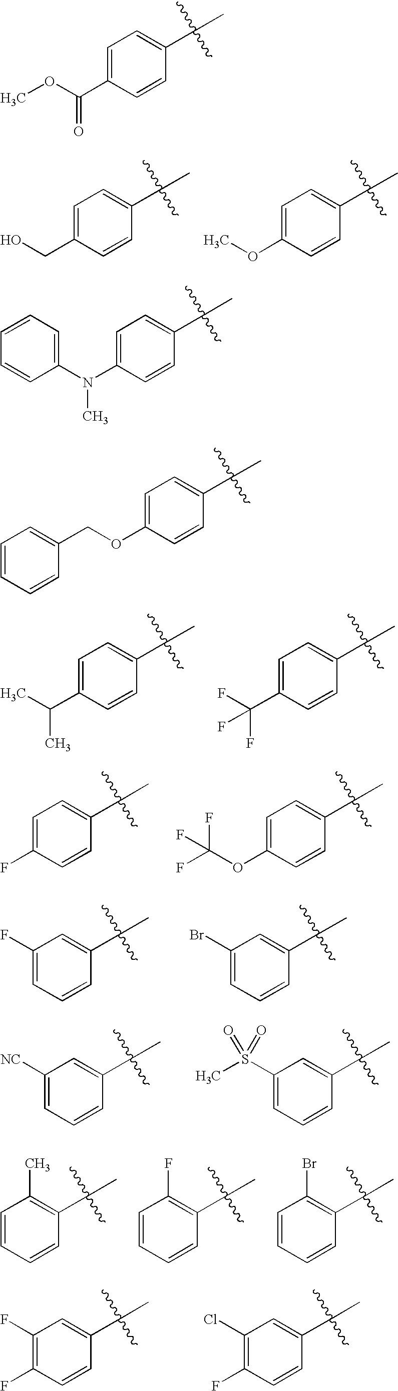

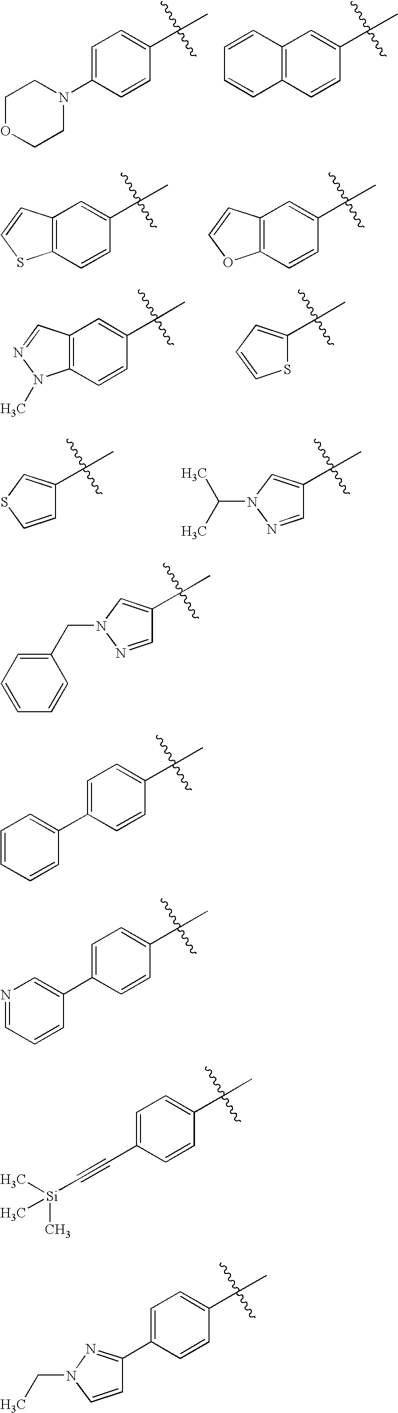

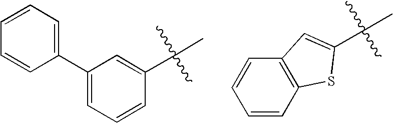

Among the compounds represented by the above-mentioned formula (1), those represented by the above-mentioned formula (1A) are preferable. In the above-mentioned formula (1A), as the combination of Ar¹, Ar², Ar³, R¹, R², R³, R⁴, R⁵, R^{6a}, R^{6b}, R^{6c}, R⁷, R⁸, n, m, and p, the combination of the preferred groups mentioned above for each is preferable. The combination of the groups mentioned as especially preferable is more preferable.

Among the compounds represented by the above-mentioned formula (1) or formula (1A), those exemplified in the following examples (Compound No. 1 to 161) are mentioned as the preferred compounds. In addition, the compounds exemplified in Table 1 below (Compound No. 162 to 264) are also preferable. Hereinafter, the compounds of the present invention are referred to as the compounds represented by formula (1) as the concept including the compounds represented by formula (1A).

TABLE 1

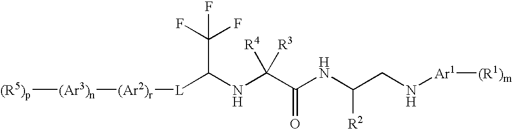

| Com- pound No. | 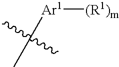 | 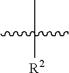 | 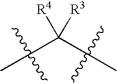 | 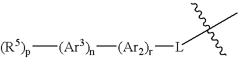 |
|---|---|---|---|---|
| 162 | 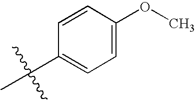 | 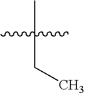 | 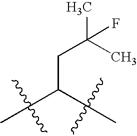 | 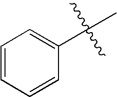 |
| 163 | 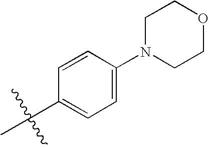 | 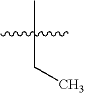 | 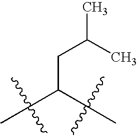 | 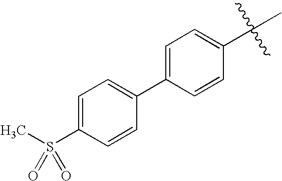 |
| 164 | 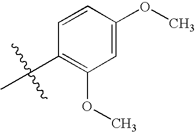 | 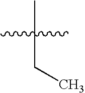 | 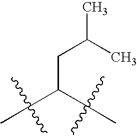 | 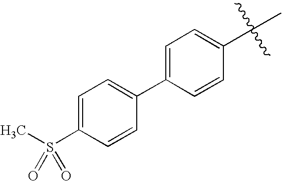 |
| 165 | 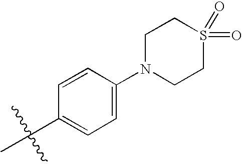 | 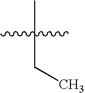 | 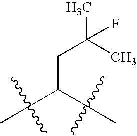 | 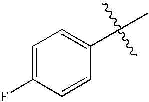 |
| 166 | 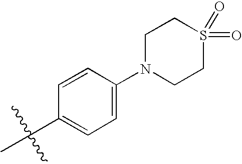 | 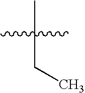 | 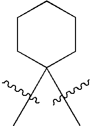 | 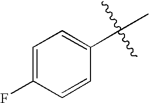 |
| 167 | 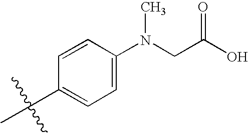 | 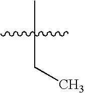 | 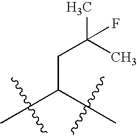 | 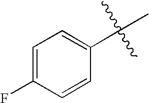 |
| 168 | 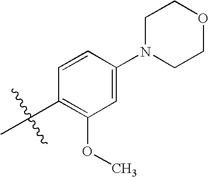 | 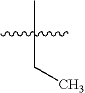 | 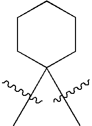 | 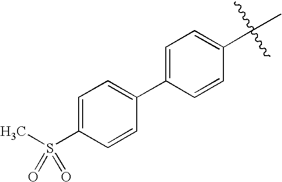 |
| 169 | 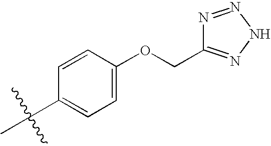 | 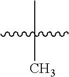 | 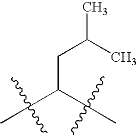 | 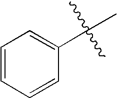 |
| 170 | 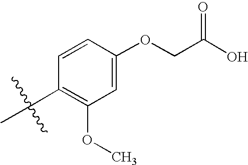 | 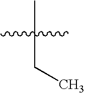 | 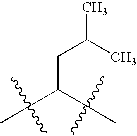 | 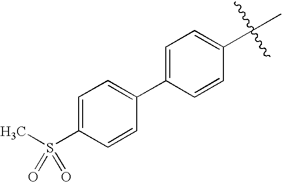 |
| 171 | 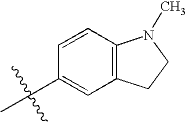 | 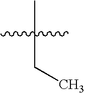 | 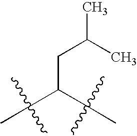 | 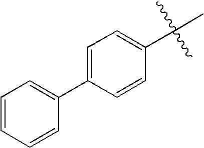 |
| 172 | 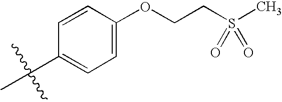 | 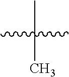 | 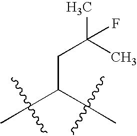 | 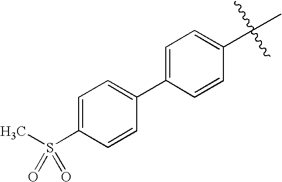 |
| 173 | 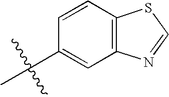 | 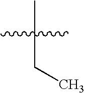 | 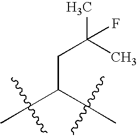 | 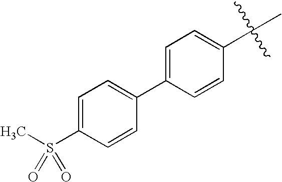 |
| 174 | 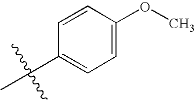 | 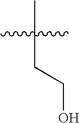 | 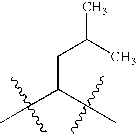 | 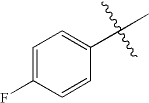 |
| 175 | 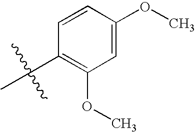 | 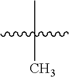 | 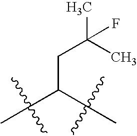 | 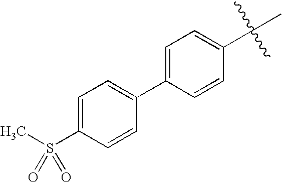 |
| 176 | 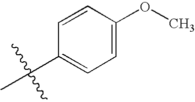 | 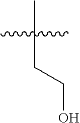 | 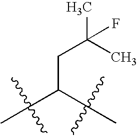 | 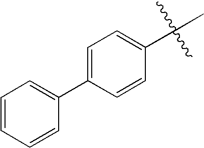 |
| 177 | 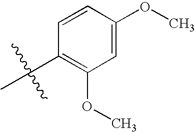 | 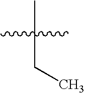 | 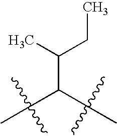 | 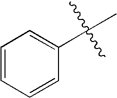 |
| 179 | 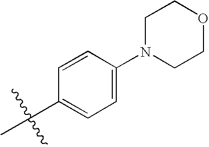 | 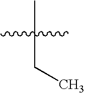 | 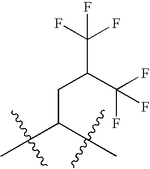 | 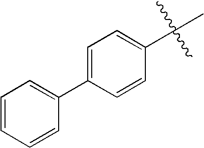 |
| 180 | 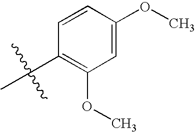 | 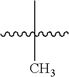 | 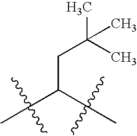 | 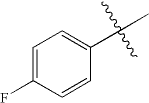 |
| 181 | 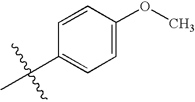 | 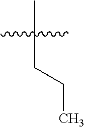 | 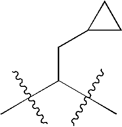 | 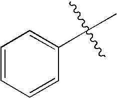 |
| 182 | 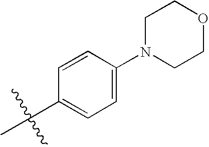 | 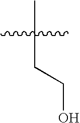 | 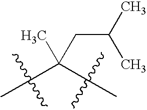 | 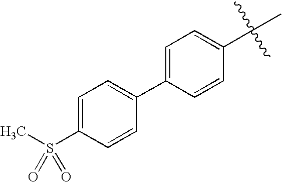 |
| 183 | 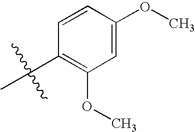 | 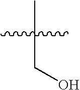 | 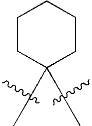 | 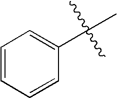 |
| 184 | 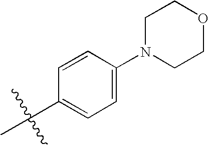 | 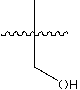 | 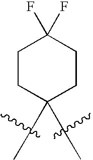 | 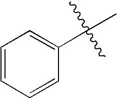 |
| 185 | 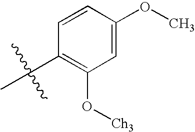 | 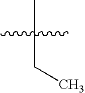 | 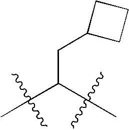 | 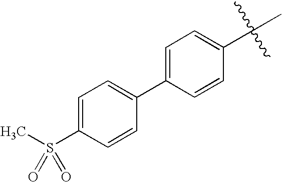 |
| 186 | 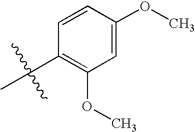 | 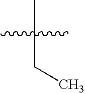 | 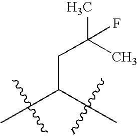 | 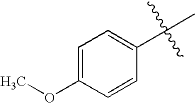 |
| 187 | 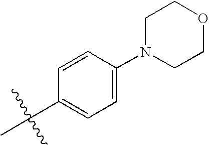 | 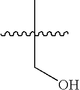 | 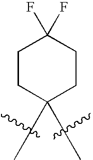 | 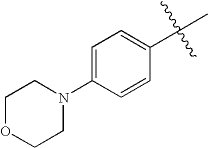 |
| 188 | 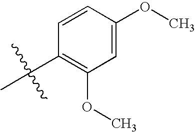 | 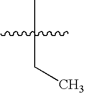 | 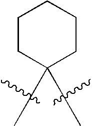 | 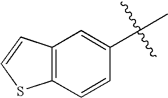 |
| 189 | 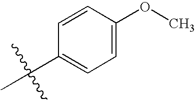 | 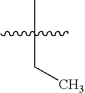 | 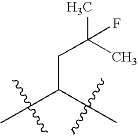 | 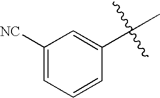 |
| 190 | 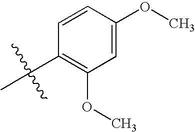 | 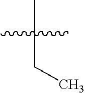 | 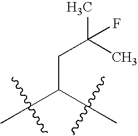 | 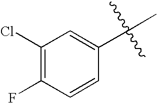 |
| 191 | 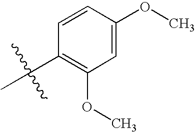 | 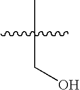 | 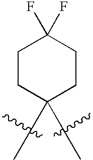 | 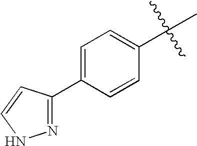 |
| 192 | 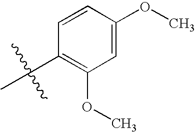 | 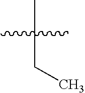 | 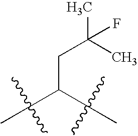 | 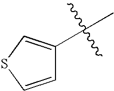 |
| 193 | 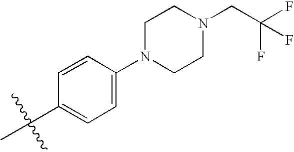 | 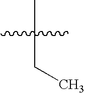 | 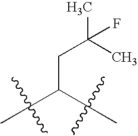 | 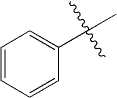 |
| 194 | 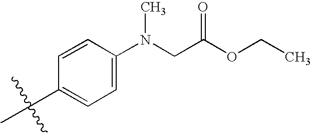 | 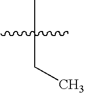 | 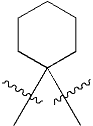 | 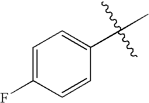 |
| 195 | 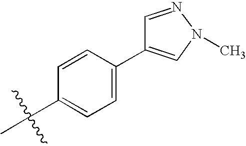 | 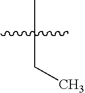 | 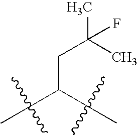 | 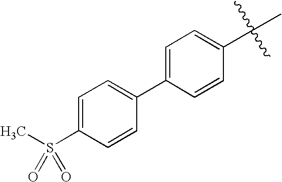 |
| 196 | 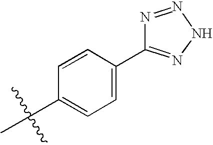 | 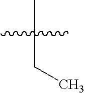 | 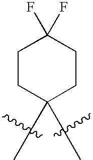 | 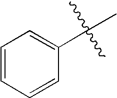 |
| 197 | 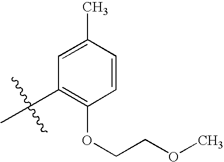 | 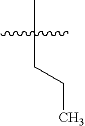 | 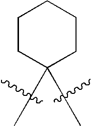 | 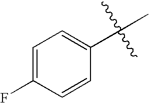 |
| 198 | 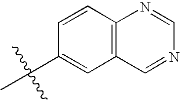 | 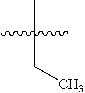 | 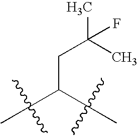 | 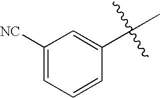 |
| 199 | 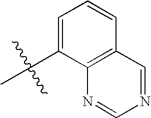 | 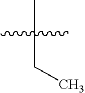 | 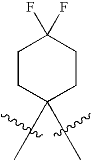 | 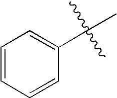 |
| 200 | 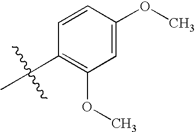 | 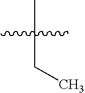 | 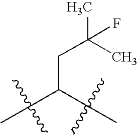 | 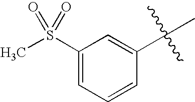 |
| 201 | 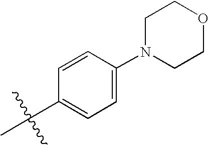 | 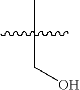 | 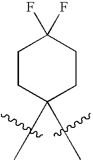 | 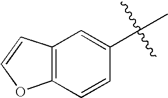 |
| 202 | 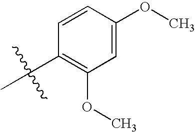 | 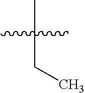 | 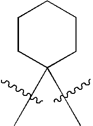 | 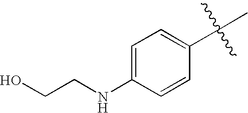 |
| 203 | 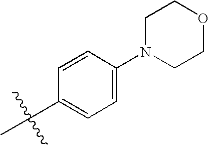 | 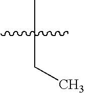 | 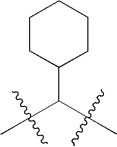 | 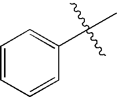 |
| 204 | 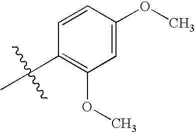 | 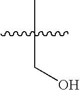 | 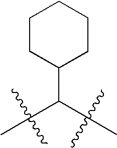 | 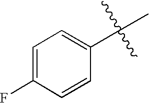 |
| 205 | 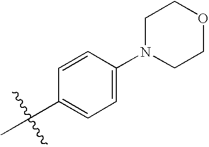 | 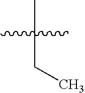 | 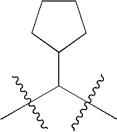 | 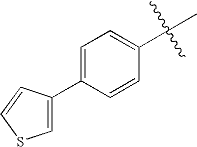 |
| 206 | 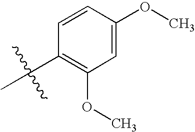 | 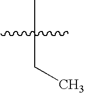 | 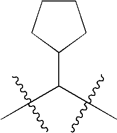 | 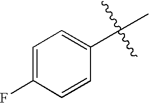 |
| 207 | 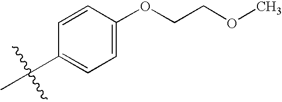 | 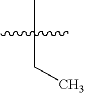 | 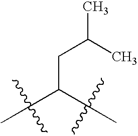 | 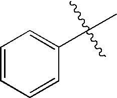 |
| 208 | 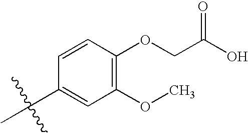 | 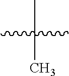 | 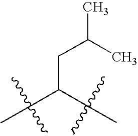 | 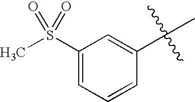 |
| 209 | 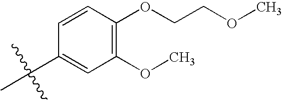 | 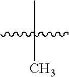 | 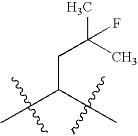 | 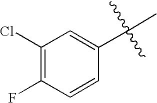 |
| 210 | 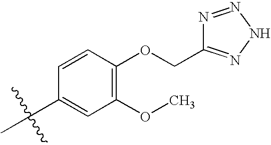 | 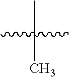 | 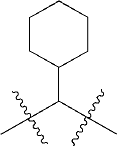 | 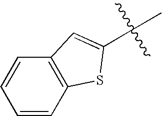 |
| 211 | 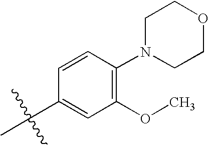 | 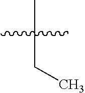 | 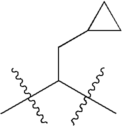 | 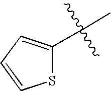 |
| 212 | 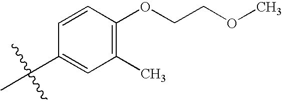 | 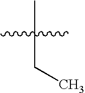 | 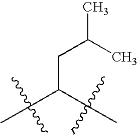 | 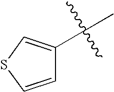 |
| 213 | 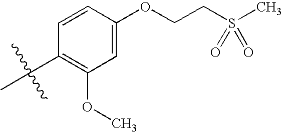 | 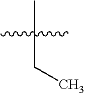 | 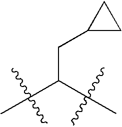 | 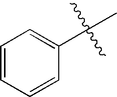 |
| 214 | 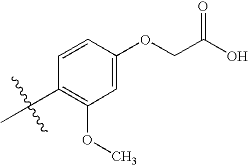 | 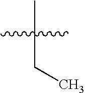 | 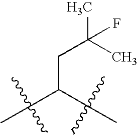 | 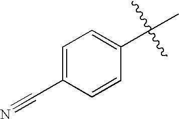 |
| 215 | 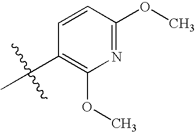 | 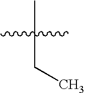 | 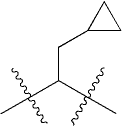 | 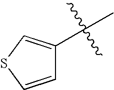 |
| 216 | 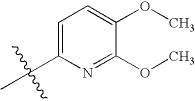 | 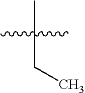 | 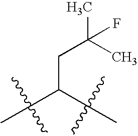 | 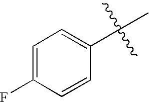 |
| 217 | 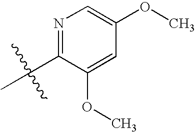 | 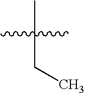 | 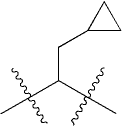 | 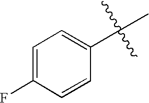 |
| 218 | 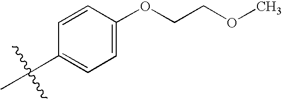 | 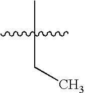 | 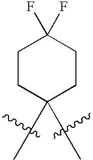 | 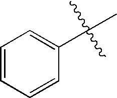 |
| 219 | 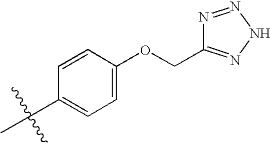 | 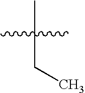 | 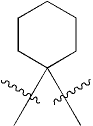 | 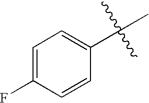 |
| 220 | 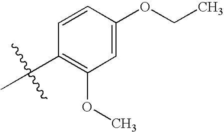 | 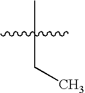 | 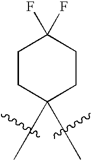 | 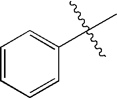 |
| 221 | 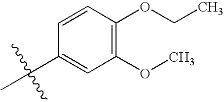 | 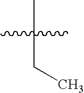 | 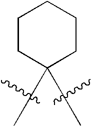 | 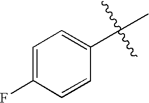 |
| 222 | 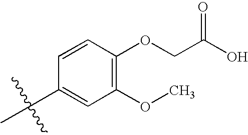 | 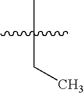 | 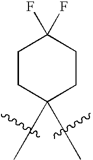 | 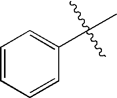 |
| 223 | 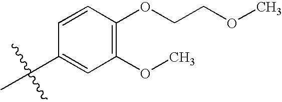 | 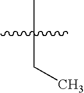 | 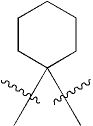 | 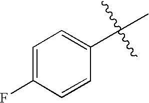 |
| 224 | 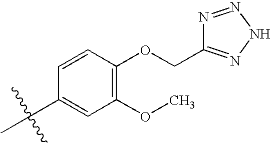 | 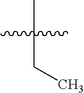 | 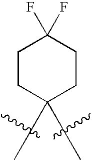 | 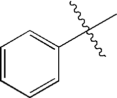 |
| 225 | 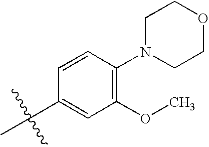 | 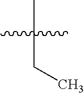 | 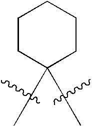 | 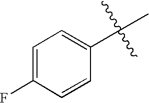 |
| 226 | 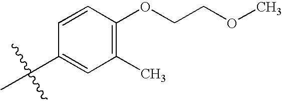 | 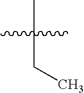 | 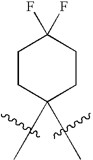 | 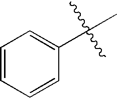 |
| 227 | 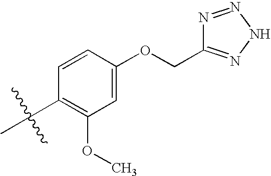 | 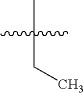 | 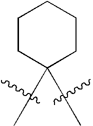 | 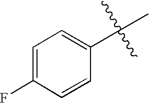 |
| 228 | 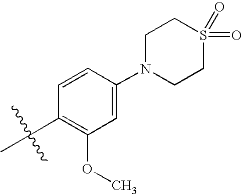 | 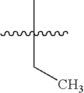 | 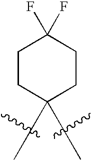 | 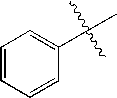 |
| 229 | 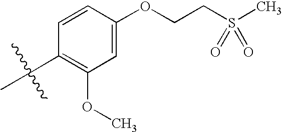 | 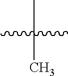 | 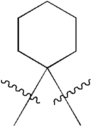 | 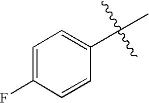 |
| 230 | 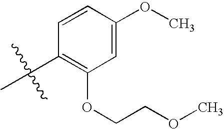 | 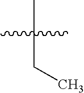 | 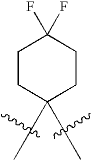 | 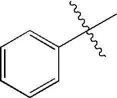 |
| 231 | 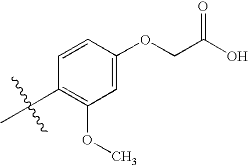 | 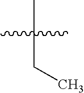 | 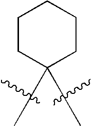 | 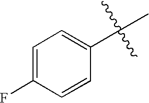 |
| 232 | 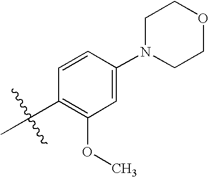 | 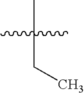 | 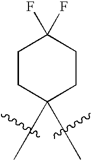 | 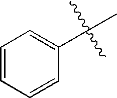 |
| 233 | 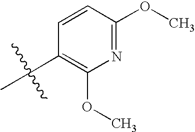 | 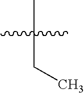 | 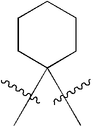 | 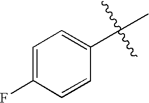 |
| 234 | 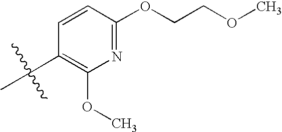 | 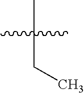 | 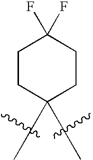 | 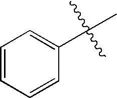 |
| 235 | 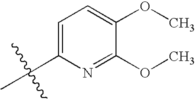 | 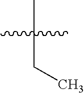 | 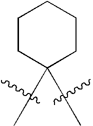 | 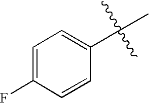 |
| 236 | 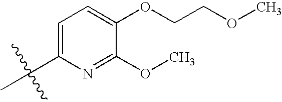 | 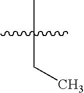 | 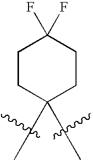 | 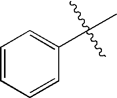 |
| 237 | 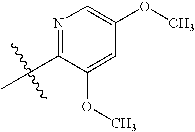 | 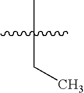 | 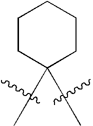 | 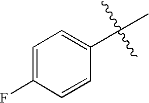 |
| 238 | 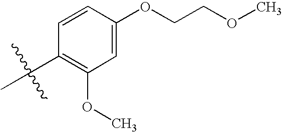 | 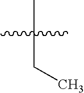 | 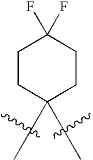 | 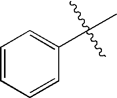 |
| 239 | 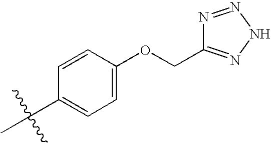 | 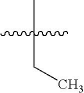 | 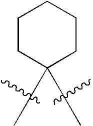 | 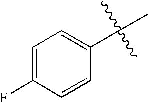 |
| 240 | 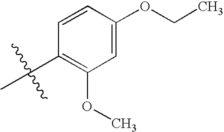 | 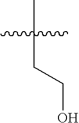 | 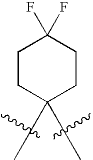 | 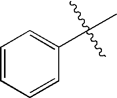 |
| 241 | 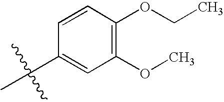 | 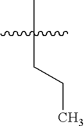 | 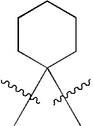 | 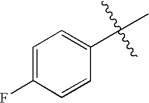 |
| 242 | 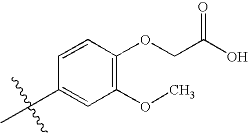 | 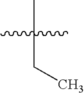 | 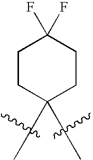 | 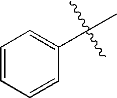 |
| 243 | 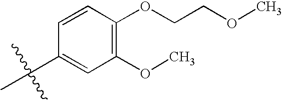 | 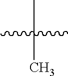 | 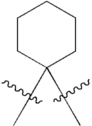 | 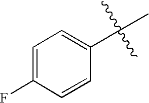 |
| 244 | 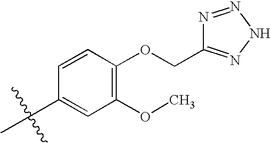 | 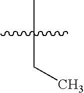 | 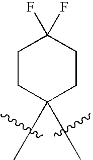 | 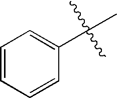 |
| 245 | 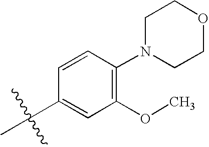 | 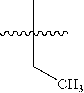 | 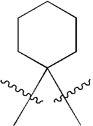 | 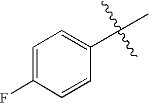 |
| 246 | 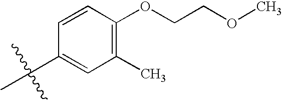 | 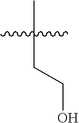 | 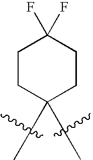 | 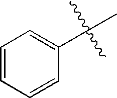 |
| 247 | 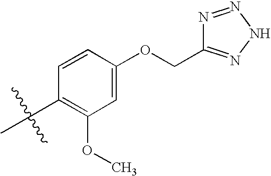 | 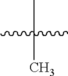 | 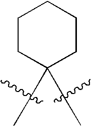 | 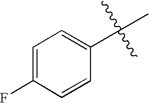 |
| 248 | 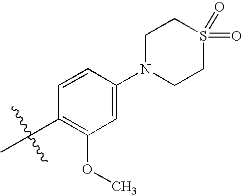 | 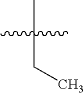 | 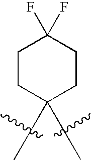 | 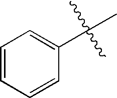 |
| 249 | 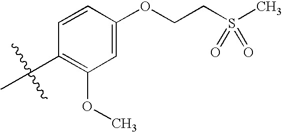 | 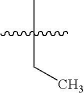 | 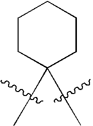 | 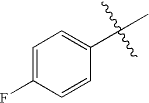 |
| 250 | 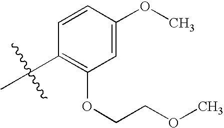 | 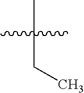 | 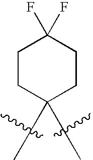 | 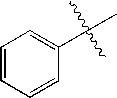 |
| 251 | 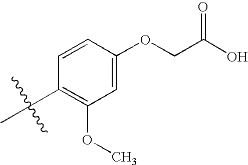 | 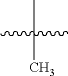 | 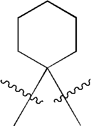 | 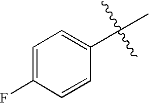 |
| 252 | 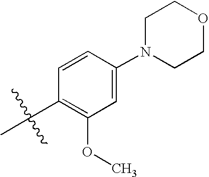 | 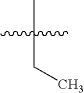 | 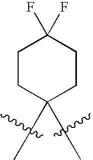 | 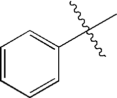 |
| 253 | 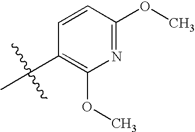 | 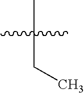 | 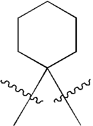 | 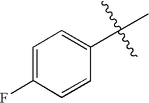 |
| 254 | 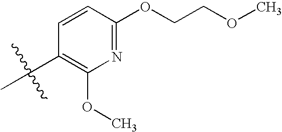 | 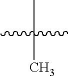 | 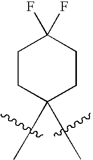 | 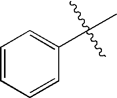 |
| 255 | 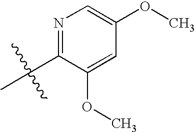 | 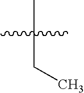 | 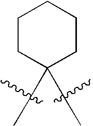 | 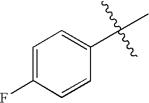 |
| 256 | 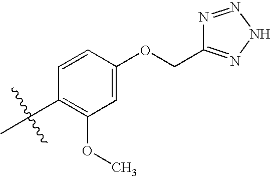 | 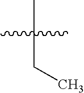 | 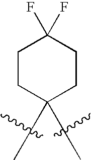 | 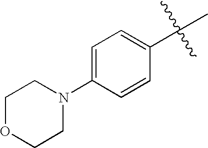 |
| 257 | 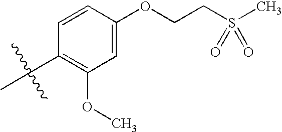 | 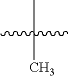 | 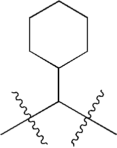 | 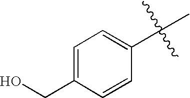 |
| 258 | 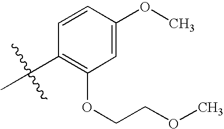 | 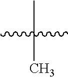 | 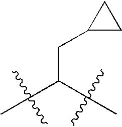 | 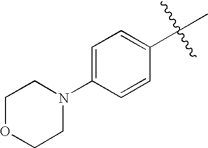 |
| 259 | 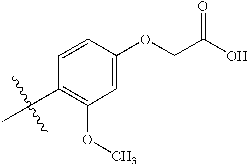 | 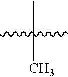 | 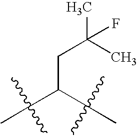 | 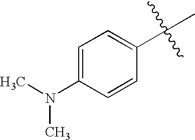 |
| 260 | 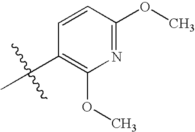 | 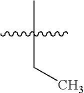 | 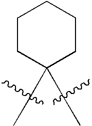 | 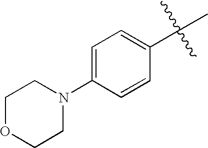 |
| 261 | 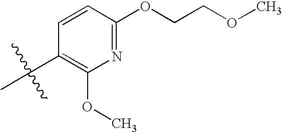 | 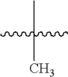 | 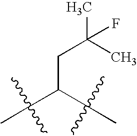 | 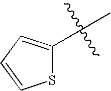 |
| 262 | 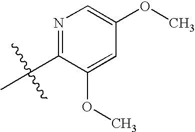 | 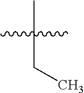 | 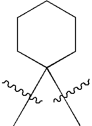 | 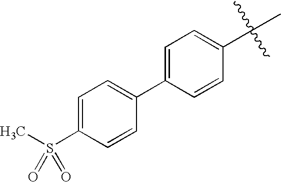 |
| 263 | 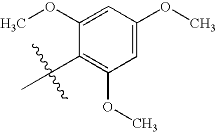 | 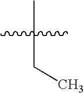 | 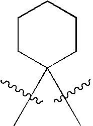 | 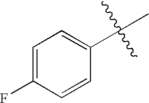 |
| 264 | 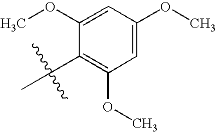 | 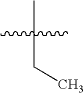 | 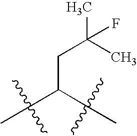 | 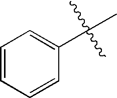 |

### General Synthetic Method

The compounds and their intermediates of the present invention can synthesized according to, for example, any of the synthetic methods described below. In each formula, Ar¹, Ar², Ar³, L, R¹, R², R³, R⁴, R⁵, m, n, p, and r are as defined in formula (1). In addition, the reagents, solvents or the like as the reaction conditions described in the chemical formulae are only for exemplification as described also in the present text. Each substituent may be protected by an appropriate protection group as needed, and may be deprotected at appropriate stage. Note that, as appropriate protection groups and methods of removal of the protection group, a protection group for each substituent widely used in this field and a known method can employed (Reference Literature: Protective Groups in Organic Synthesis, Third Edition, John Wiley & Sons, Inc.).

In addition, when abbreviation of the substituent, reagent, and solvent is used in the present text or Tables, the abbreviation each represents the followings.

HATU: O-(7-azabenzotriazol-1-yl)-N,N,N′,N′-tetramethyluronium hexafluorophosphate
PyBOP: benzotriazol-1-yloxytris(pyrrolidino)phosphonium hexafluorophosphate
X-Phos: 2-(di-tert-butylphosphino)-2′,4′,6′-triisopropyl-1,1′-biphenyl

### DMF: N,N-dimethylformamide

THF: tetrahydrofuran
Ph: phenyl
TFA: trifluoroacetic acid

### (1) Synthesis of Compound of Formula (7)

The compound of formula (7) may be synthesized according to the method described in, for example, US Patent Publication 2006/030731 and the like.

That is, first, the aminoacetic acid ester derivative of formula (4) is reacted with the ketone derivative of formula (5) to synthesize the imine intermediate of formula (6). Then, by reacting the imine intermediate of formula (6) with an appropriate reducing agent, the compound of formula (7) is synthesized. The ketone derivative of formula (5) cam be synthesized referring to, for example, Tetrahedron, 2006, 62, 5092-5098; Angew. Chem. Int. Ed., 1998, 37, 6, 820-821; and the like.

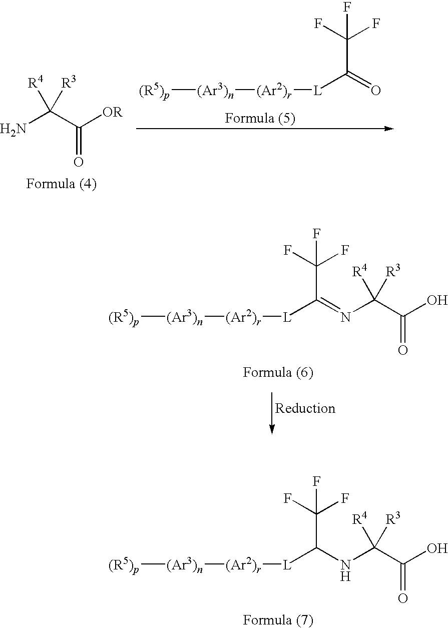

The compound of formula (7) may also be synthesized according to the method described in WO2003/075836; J. Org. Chem. 2006, 71, 4320-4323; Bioorg. Med. Chem. Lett., 2008, 18, 923-928; and the like.

That is, first, the amine derivative of formula (8) with a hydroxyl protected by an appropriate protection group is reacted with trifluoroacetaldehyde to synthesize the imine intermediate of formula (9). Meanwhile, an organometallic reagent of formula (10) such as an organolithium reagent or a Grignard reagent is prepared according to the common method. By reacting the organometallic reagent of formula (10) with the imine intermediate of formula (9), the intermediate of formula (11) is synthesized. By subsequent removal of the protection group P from the hydroxyl and oxidation, the compound of formula (7) is synthesized.

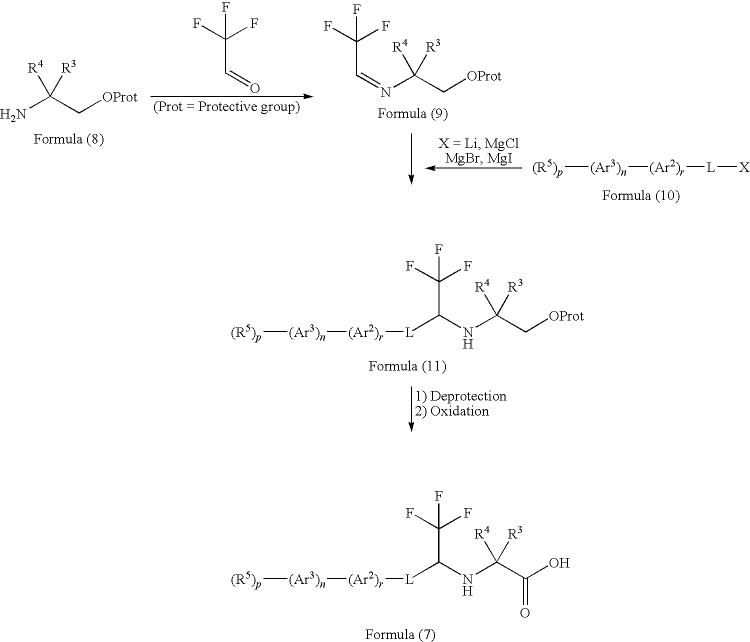

(2) Synthesis of the Compound of Formula (1) from the Compound of Formula (7)

### (Route A)

By reacting the compound of formula (7) with the amine derivative of formula (12) in the presence of appropriate activating agent of a carboxyl (for example, HATU or PyBOP) and in the presence or absence of appropriate base (for example, triethylamine or N-ethyl-N,N-diisopropylamine) and in an appropriate organic solvent (for example, DMF or THF) in a temperature range from 0° C. to the heat-reflux temperature of the solvent, the compound of formula (1) is synthesized.

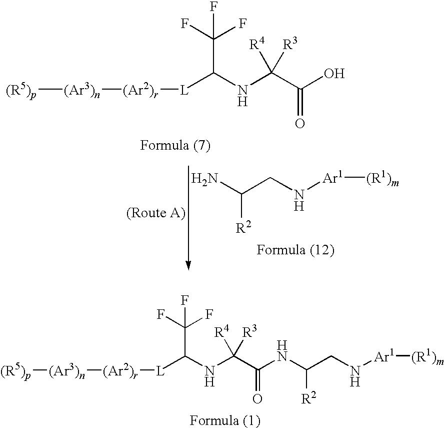

### (Route B)

By reacting the compound of formula (7) with an appropriately protected amine derivative represented by formula (13) in the presence of an appropriate activating agent of carboxyl (for example, HATU or PyBOP) and in the presence or absence of an appropriate base (for example, triethylamine or N-ethyl-N,N-diisopropylamine) and in an appropriate organic solvent (for example, DMF or THF) in a temperature range from 0° C. to the heat-reflux temperature of the solvent, followed by deprotection under an appropriate deprotection condition, the compound of formula (14) is synthesized. By reacting the compound of formula (14) with a reagent having a leaving group represented by formula (15) in the presence or absence of an appropriate Cu reagent (for example, copper (11) acetate), in the presence or absence of an appropriate additive (for example, myristic acid), in the presence of an appropriate base (for example, 2,6-lutidine, triethylamine, or N-ethyl-N,N-diisopropylamine) and in an appropriate organic solvent (for example, toluene, acetonitrile, DMF, or 2-propanol) or a mixed solvent thereof, in a temperature range from 0° C. to the heat-reflux temperature of the solvent, the compound represented by the formula (1) is synthesized.

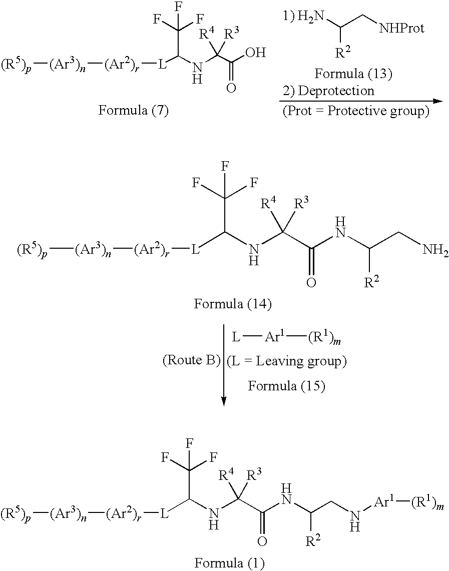

### (3) Conversion of the Compound of Formula (1) and the Compound of Formula (11)

### (Route C)

To the compound of formula (1) or the compound of formula (11), when n or r is 1 and R⁵ is bromine or iodine, by performing the Suzuki-Miyaura cross-coupling reaction, the compound of formula (1c) and formula (11c) in which the structure of R⁵ is converted into W (aryl or heteroaryl) can synthesized. That is, by reacting the compound of formula (1) or the compound of formula (11) with a boric acid reagent represented by WB(OR)₂ (in which W is an aryl or heteroaryl) in the presence of an appropriate Pd catalyst (for example, Pd₂(dba)₃) and an appropriate ligand (for example, X-Phos), or an appropriate complex of Pd catalyst and ligand (for example, PdCl₂(dppf).CH₂Cl₂), in the presence of an appropriate base (for example, cesium carbonate or potassium tert-butoxide), and in an appropriate solvent (for example, DMF, 2-propanol, or water) or a mixed solvent thereof, in a temperature range from room temperature to the heat-reflux temperature of the solvent, the compound of formula (1c) or the compound of formula (11c) is synthesized.

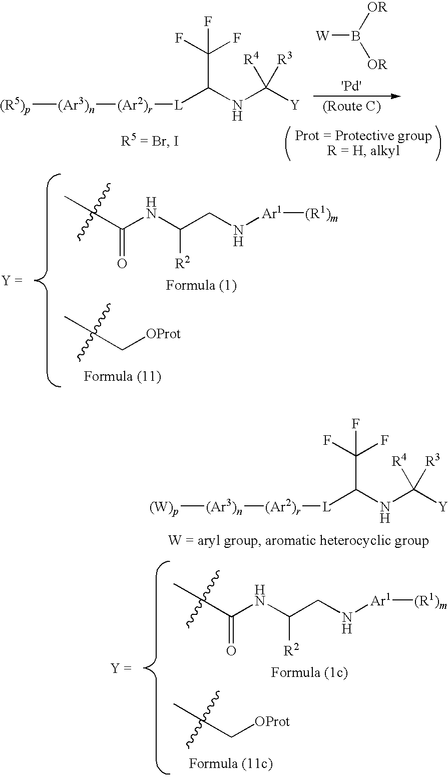

### (Route D)

To the compound of formula (1) or the compound of formula (11), when R⁵ is bromine or iodine, the compound (1d) and the compound (11d) in which the structure of R⁵ is converted into a cyano can be synthesized.

When n or r is 1, by performing the Negishi cross-coupling reaction, the structure of R⁵ can converted into a cyano. That is, by reacting the compound of formula (1) or the compound of formula (11) with an appropriate metal cyanide reagent (for example, Zn(CN)₂)) in the presence of an appropriate Pd catalyst (for example, Pd₂(dba)₃) and an appropriate ligand (for example, X-Phos), or an appropriate complex of Pd catalyst and ligand (for example, PdCl₂(dppf).CH₂Cl₂), and in an appropriate solvent (for example, DMF or THF), in a temperature range from room temperature to the heat-reflux temperature of the solvent, the compound of formula (1d) or the compound of formula (11d) is synthesized.

When n=r=0 and L is not a single bond, by reacting the compound of formula (1) or the compound of formula (11) with an appropriate metal cyanide reagent (for example, KCN) in an appropriate solvent (for example, DMF or THF) in a temperature range from room temperature to the heat-reflux temperature of the solvent, the compound of formula (1d) or the compound of formula (11d) is synthesized.

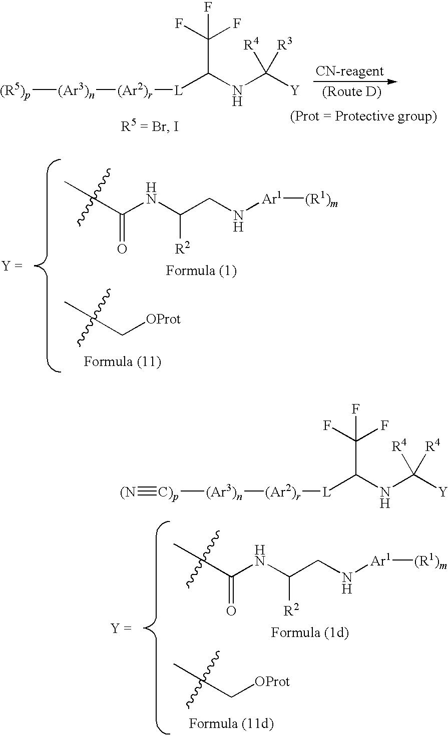

### (Route E)

To the compound of formula (1) or the compound of formula (11), when R⁵ is bromine or iodine, the compound (1e) and the compound (11e) in which the structure of R⁵ is converted into —N(R^{6a})(R^{6b}) can be synthesized.

When n or r is 1, by performing the Buchwald-Hartwig cross-coupling reaction, the structure of R⁵ can be converted into —N(R^{6a})(R^{6b}). That is, by reacting the compound of formula (1) or the compound of formula (11) with an amine represented by (R^{6a})(R^{6b})NH in the presence of an appropriate Pd catalyst (for example, Pd₂(dba)₃) and an appropriate ligand (for example, X-Phos), or an appropriate complex of Pd catalyst and ligand (for example, PdCl₂(dppf).CH₂Cl₂), in the presence of an appropriate base (for example, cesium carbonate or potassium tert-butoxide), and in an appropriate solvent (for example, toluene or DMF) or a mixed solvent thereof, in a temperature range from room temperature to the heat-reflux temperature of the solvent, the compound of formula (1e) or the compound of formula (11e) is synthesized.

When n=r=0 and L is not a single bond, by reacting the compound of formula (1) or the compound of formula (11) with an amine represented by (R^{6a})(R^{6b})NH in the presence or absence of an appropriate base (for example, N-ethyl-N,N-diisopropylamine) in an appropriate solvent (for example, DMF or THF) in a temperature range from room temperature to the heat-reflux temperature of the solvent, the compound of formula (1e) or the compound of formula (11e) is synthesized.

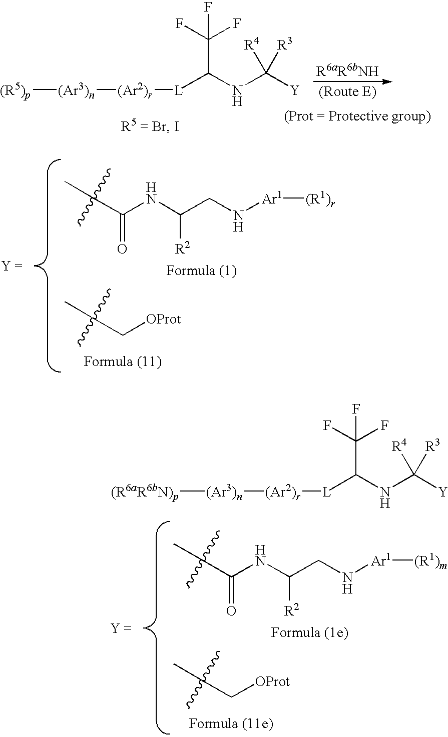

### (Route F)

To the compound of formula (1) or the compound of formula (11), when n or r is 1 and R⁵ is bromine or iodine, by performing the Sonogashira cross-coupling reaction, the compound of formula (1f) and formula (11f) in which the structure of R⁵ is converted into 1-alkynyl can be synthesized. That is, by reacting the compound of formula (1) or the compound of formula (11) with 1-alkyne in the presence of an appropriate Pd catalyst (for example, Pd₂(dba)₃) and an appropriate ligand (for example, X-Phos), or an appropriate complex of Pd catalyst and ligand (for example, PdCl₂(dppf).CH₂Cl₂), in the presence of an appropriate Cu catalyst (for example, copper(I) iodide or copper(I) bromide), and in the presence of an appropriate base (for example, triethylamine, diethylamine, or piperidine), and in an appropriate solvent (for example, DMF, THF, or triethylamine), in a temperature range from room temperature to the heat-reflux temperature of the solvent, the compound of formula (1f) or the compound of formula (11f) is synthesized.

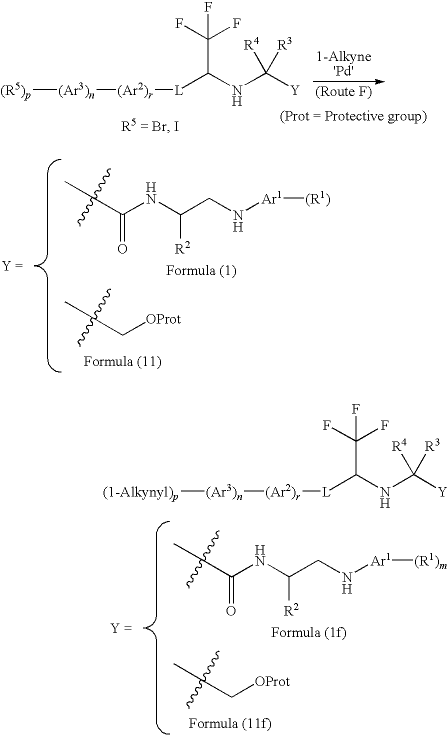

### (Route G)

To the compound of formula (1) or the compound of formula (11), when R⁵ is bromine or iodine, by performing the hydrogen reduction, the compound of formula (1g) and formula (11g) can be synthesized. That is, by reacting the compound of formula (1) or the compound of formula (11) with an appropriate hydrogen source (for example, hydrogen gas, ammonium formate, or cyclohexene) in the presence of an appropriate Pd catalyst (for example, Pd/C) and in an appropriate solvent (for example, methanol, ethanol, or tetrahydrofuran), in a temperature from room temperature to the heat-reflux temperature of the solvent, the compound of formula (1g) or the compound of formula (11g) is synthesized.

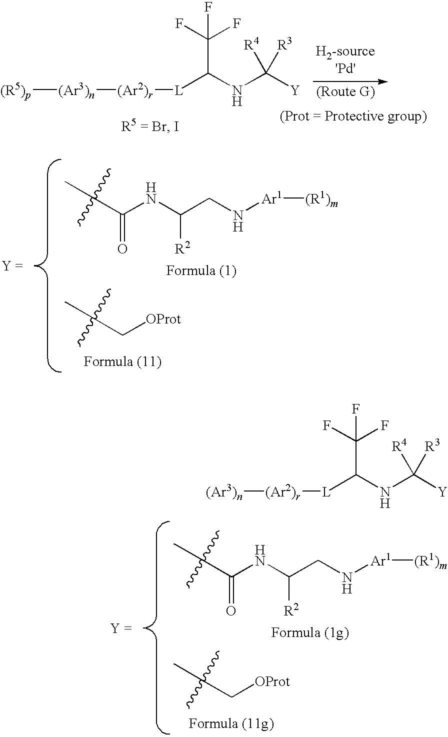

In addition, besides the conversion of the above-mentioned Route A to G, the conversion reaction that is well known to those skilled in the art can be performed to the compound of formula (1) of the present invention. For example, when the compound of formula (1) of the present invention has a substituent(s) which is easily convertible, such as —O(CO)R^{6a}, —COOR^{6a}, or nitro, each substituent can be converted by performing the reaction well known to those skilled in the art. That is, for example, —O(CO)R^{6a} can be converted into hydroxyl, —COOR^{6a} into carboxyl or hydroxymethyl, and nitro into amino.

When the compound of formula (1) of the present invention has carboxyl, the compound can converted into the compound of formula (1) of the present invention having a substituent(s) such as —COOR^{6a} and —CON(R^{6a})(R^{6b}) by the reaction well known to those skilled in the art.

When the compound of formula (1) of the present invention has a hydroxyl, the compound can converted into the compound of formula (1) of the present invention having a substituent(s) such as —OR^{6a} and —O(CO)R^{6a} by the reaction well known to those skilled in the art.

When the compound of formula (1) of the present invention has amino, the compound can converted into the compound of formula (1) having a substituent such as N(R^{6a})(R^{6b}), —NR^{6a}(CO)R^{6b}, —NR^{6a}(CO)N(R^{6b})(R^{6c}), and —NR^{6a}S(O)₂R^{6b} by the reaction well known to those skilled in the art.

When the compound of formula (1) of the present invention has cyano, the compound can converted into the compound of formula (1) of the present invention having a substituent such as triazolyl and tetrazolyl by the reaction well known to those skilled in the art.

The present invention also relates to the pharmaceutically acceptable salt of the compound represented by formula (1). Examples of such salt include a salt with an inorganic acid such as hydrogen chloride, hydrogen bromide, sulfuric acid, nitric acid, phosphoric acid, and carbonic acid; a salt with an organic acid such as maleic acid, fumaric acid, citric acid, malic acid, tartaric acid, lactic acid, succinic acid, benzoic acid, oxalic acid, methanesulfonic acid, benzenesulfonic acid, p-toluenesulfonic acid, acetic acid, trifluoroacetic acid, and formic acid; a salt with an amino acid such as glycine, lysine, arginine, hisitidine, ornithine, glutamic acid, and aspartic acid; a salt with an alkali metal such as sodium, potassium, and lithium; a salt with an alkali earth metal such as calcium and magnesium; a salt with a metal such as aluminum, zinc, and iron; a salt with an organic onium such as tetramethylammonium and choline; and a salt with an organic base such as ammonia, propanediamine, pyrrolidine, piperidine, pyridine, ethanolamine, N,N-dimethylethanolamine, 4-hydroxypiperidine, t-octylamine, dibenzylamine, morpholine, glucosamine, phenylglycylalkyl ester, ethylenediamine, N-methylglucamine, guanidine, diethylamine, triethylamine, dicyclohexylamine, N,N′-dibenzylethylenediamine, chloroprocaine, procaine, diethanolamine, N-benzylphenylamine, piperazine, and tris(hydroxymethyl)aminomethane.

The above-mentioned various pharmaceutically acceptable salts of the compound represented by formula (1) can be appropriately produced based on the ordinary knowledge of such technical field.

The compound of the present invention includes the stereoisomer, racemate, and all possible optically active substances of the compound represented by formula (1). In addition, the compound of the present invention may form tautomer depending on the combination of each substituent. Such tautomers are also included in the compound of the present invention. Examples of the combination of the substituent which forms such tautomer include, but are not limited to, the following structure.

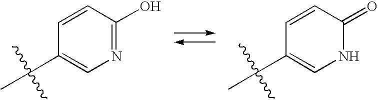

The compound represented by formula (1) of the present invention and the pharmaceutically acceptable salt thereof have excellent cysteine protease inhibitory effect, especially excellent cathepsin K inhibitory effect. Due to its excellent cysteine protease inhibitory effect, the compound represented by formula (1) of the present invention and the pharmaceutically acceptable salt thereof are useful as cysteine protease inhibitors (especially cathepsin K inhibitors).

The compound represented by formula (1) of the present invention and the pharmaceutically acceptable salt thereof can be used as drugs clinically applicable as a cathepsin K inhibitor for treatment and prevention of the disease selected from a group consisting of osteoporosis, osteoarthritis, chronic rheumatoid arthritis, Paget's disease of bone, hypercalcemia, bone metastasis of cancer, and ostealgia.

The compound represented by the above-mentioned formula (1) and the pharmaceutically acceptable salt thereof can be used to prepare a pharmaceutical composition along with a pharmaceutically acceptable carrier and/or diluent. The pharmaceutical composition can be formed into various formulations for oral or parenteral administration. Examples of a parenteral administration include venous, subcutaneous, intramuscular, percutaneous, or intrarectal administration.

The drug formulation containing one or more of the compound represented by formula (1) of the present invention or the pharmaceutically acceptable salt thereof as an active ingredient is prepared using a carrier, diluent, or other additives which are usually used for drug formulation. As a carrier or diluent for drug formulation, any of solid and liquid may be used, examples of which include lactose, magnesium stearate, starch, talc, gelatin, agar, pectin, gum Arabic, olive oil, sesame oil, cacao butter, ethyleneglycol, and others in common use. Administration may be done in any form of oral administration of tablet, ball, capsule, granule, powder, liquid, and the like, parenteral administration by injection such as venous or intramuscular injection and the like, suppository, percutaneous administration, and others.

The compound represented by formula (1) of the present invention and the pharmaceutically acceptable salt thereof have good properties as a drug in safety, stability, pharmaceutical effect, sustainability of the action, physical properties, pharmacokinetics, preservative property, producibility, and the like.

The compound represented by formula (1) of the present invention or the pharmaceutically acceptable salt thereof can be administered usually in the range of 0.1 to 1,000 mg, preferably in the range of 1 to 100 mg, per day for adult, dividing the dosage into one or several times, although the dosage varies according to the kind of disease, administration route, or symptom, age, sex, or body weight of the patient, and the like. However, since the dosage varies according to various conditions, the smaller dosage than the above-mentioned may be sufficient in some cases and the dosage exceeding the above range may be necessary in other cases. In the case of intravenous administration, the dosage is desirably administered in a range of 0.01 to 100 mg, preferably 0.1 to 10 mg, per day for adult, dividing the dosage into one or several times, depending on the symptom.

### EXAMPLES

Hereinafter the present invention will be explained based on specific examples. However, the present invention is not limited to these examples.

The structure of the novel compound isolated was identified by ¹H-NMR and/or mass spectrometry using single quadrupole instrumentation equipped with an electron spray source, and other appropriate analytical methods.

As for the compound which ¹H-NMR spectrum (400 MHz, DMSO-d₆ or CDCl₃) was measured, its chemical shift (δ: ppm) and coupling constant (J: Hz) are shown. As for the result of mass spectroscopy, the observed value of M⁺+H, that is the value of the molecular mass of the compound (M) with a proton (H⁺) added is shown. In addition, the following abbreviation each represents the followings. s=singlet, d=doublet, t=triplet, q=quartet, brs=broad singlet, m=multiplet.

### Reference Example 1

### Synthesis of (2S)-2-[((1S)-2,2,2-trifluoro-1-{4-[4-(methylsulfonyl)phenyl]phenyl}ethyl)amino]-4-fluoro-4-methylpentanoic acid (Reference Example Compound 1)

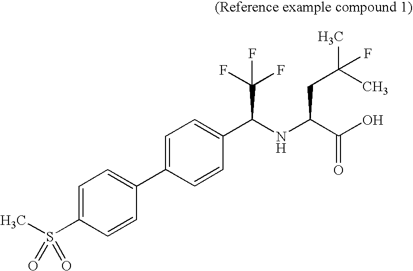

### Reference Example Compound 1

Reference example compound 1 was synthesized according to the method described in the literature (WO2003/075836 and J. Org. Chem., 2006, 71, 4320-4323), using benzyl N-(tert-butoxycarbonyl)-L-aspartate as a starting material.

¹H-NMR (400 MHz, CDCl₃) δ (ppm): 8.02 (d, J=8.0 Hz, 2H), 7.76 (d, J=8.0 Hz, 2H), 7.63 (d, J=8.0 Hz, 2H), 7.51 (d, J=8.0 Hz, 2H), 4.30 (q, J=7.0 Hz, 1H), 3.68 (dd, J=8.0, 4.1 Hz, 1H), 3.10 (s, 3H), 2.26-2.10 (m, 1H), 2.07-1.90 (m, 1H), 1.50 (d, J=8.0 Hz, 3H), 1.44 (d, J=8.0 Hz, 3H).

ESI/MS m/e: 462.0 (M⁺+H, C₂₁H₂₃F₄NO₄S).

### Reference Example 2

### Synthesis of (2S)-2-[{(1S)-2,2,2-trifluoro-1-(4-bromophenyl)ethyl}amino]-4-fluoro-4-methylpentanoic acid (Reference Example Compound 2)

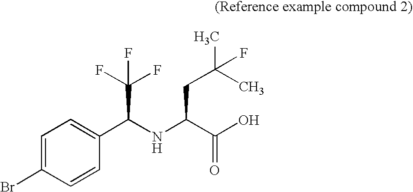

### Reference Example Compound 2

The reference example compound 2 was synthesized according to the method described in Bioorg. Med. Chem. Lett., 2008, 18, 923-928, using benzyl N-(tert-butoxycarbonyl)-L-aspartate as a starting substance.

¹H-NMR (400 MHz, CDCl₃) δ (ppm): 7.52 (2H, dt, J=8.9, 2.1 Hz), 7.26 (2H, t, J=4.3 Hz), 4.18 (1H, q, J=7.0 Hz), 3.65 (1H, dd, J=7.8, 4.4 Hz), 2.16 (1H, ddd, J=23.3, 15.0, 4.4 Hz), 1.96 (1H, dq, J=20.7, 6.1 Hz), 1.46 (6H, dd, J=21.7, 9.5 Hz).

ESI/MS m/e: 387.2 (M⁺+H, C₁₄H₁₆BrF₄NO₂).

### Reference Example 3

### Synthesis of 1-[(2,2,2-trifluoro-1-phenylethyl)amino]cyclohexanecarboxylic acid (Reference Example Compound 3)

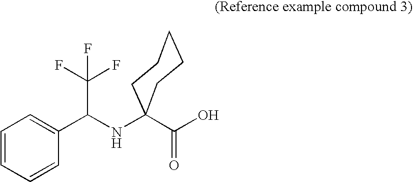

### Reference Example Compound 3

1-Aminocyclohexanecarboxylic acid methyl ester (157 mg) was dissolved in methanol (2.0 mL) and then potassium carbonate (138 mg) and 2,2,2-trifluoroacetophenone (154 μL) were added. The mixture solution was heated while stirring at 50° C. for 18 hours. The reaction solution was cooled to room temperature and the insoluble matter was separated by filtration. The filtrate was concentrated and the residue was washed with diethyl ether to obtain the crude product of imine intermediate.

The crude product was suspended in THF (6.4 mL) and sodium tetrahydroborate (151 mg) and water (0.26 mL) were added. The mixture solution was stirred at room temperature for 18 hours and then heated while stirring at 60° C. for 3 hours. The reaction solution was cooled to room temperature and the reaction was quenched with aqueous 1 mol/L sodium hydroxide solution (12 mL). To the solution, hexane (3 mL) was added and the separated organic layer was removed. After adding 2 mol/L hydrochloric acid (12 mL) to the aqueous layer, sodium chloride was added until the aqueous solution was saturated, and then extraction was performed with ethyl acetate. The organic layer was dried over anhydrous sodium sulfate and filtered. The filtrate was concentrated in vacuo to obtain the crude product of the title compound (Reference example compound 3: 120 mg). The crude product was used for the subsequent reaction without further purification.

¹H-NMR (400 MHz, DMSO-d₆) δ (ppm): 12.10 (brs, 1H), 7.55-7.25 (m, 5H), 6.53 (s, 1H), 4.44 (m, 2H), 2.92 (brs, 1H), 1.05-2.05 (m, 10H).

ESI/MS m/e: 302.1 (M⁺+H, C₁₅H₁₈F₃NO₂).

### Reference Example 4

### Synthesis of ((2S)-2-aminobutyl)(4-methoxyphenyl)amine (Reference Example Compound 4)

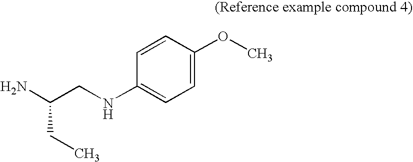

### Reference Example Compound 4

The reference example compound 4 was synthesized according to the method described in the literature (Bioorg. Med. Chem., 2006, 14, 6789-6806), using 4-methoxyaniline as a starting material, and obtained as a hydrochloride.

ESI/MS m/e: 195.1 (M⁺+H, C₁₁H₁₈N₂O).

### Reference Example 5

### Synthesis of ((2S)-2-amino-3-benzyloxypropyl)(4-methoxyphenyl)amine (Reference Example Compound 5)

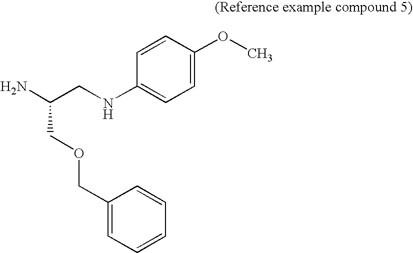

### Reference Example Compound 5

The reference example compound 5 was synthesized according to the method described in the literature (Bioorg. Med. Chem., 2006, 14, 6789-6806), using 4-methoxyaniline and (R)-(+)-3-benzyloxy-2-{(tert-butoxy)carbonylamino}-1-propanol as a starting material, and obtained as a hydrochloride.

ESI/MS m/e: 287.1 (M⁺+H, C₁₇H₂₂N₂O₂).

### Reference Example 6

### Synthesis of {(2S)-2-amino-3-(tert-butyldimethylsiloxy)propyl}(4-methoxyphenyl)amine (Reference Example Compound 6)

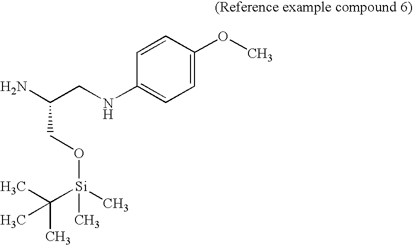

### Reference Example Compound 6

The reference example compound 6 was synthesized referring to the literature (Bioorg. Med. Chem., 2006, 14, 6789-6806), using 4-methoxyaniline and (R)-(+)-N-(tert-butoxycarbonyl)-O-(tert-butyldimethylsilyl)serinol as a starting material, and obtained as a free base using trifluoroacetic acid instead of hydrogen chloride.

ESI/MS m/e: 311.2 (M⁺+H, C₁₆H₃₀N₂O₂Si).

### Reference Example 7

### Synthesis of ((2S)-2-aminobutyl)(2,4-dimethoxyphenyl)amine (Reference Example Compound 7)

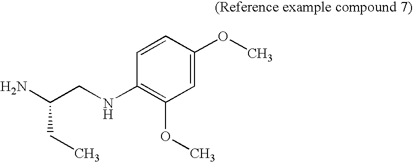

### Reference Example Compound 7

The reference example compound 7 was synthesized according to the method described in the literature (Bioorg. Med. Chem., 2006, 14, 6789-6806), using 2,4-dimethoxyaniline as a starting material, and obtained as a hydrochloride.

ESI/MS m/e: 225.1 (M⁺+H, C₁₂H₂₀N₂O₂).

### Reference Example 8

### Synthesis of ((2S)-2-aminobutyl)(3,4-diethoxyphenyl)amine (Reference Example Compound 8)

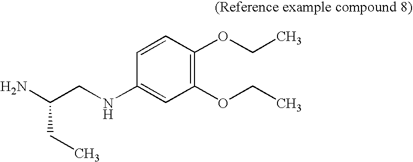

### Reference Example Compound 8

The reference example compound 8 was synthesized according to the method described in the literature (Bioorg. Med. Chem., 2006, 14, 6789-6806), using 3,4-diethoxyaniline as a starting material, and obtained as a hydrochloride.

ESI/MS m/e: 253.2 (M⁺+H, C₁₄H₂₄N₂O₂).

### Reference Example 9

### Synthesis of ((2S)-2-aminobutyl)(4-morpholin-4-ylphenyl)amine (Reference Example Compound 9)

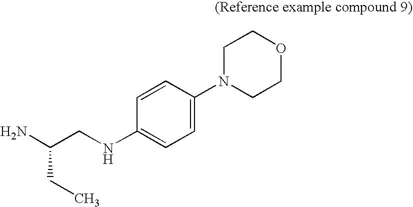

### Reference Example Compound 9

The reference example compound 9 was synthesized according to the method described in the literature (Bioorg. Med. Chem. Lett., 2006, 16, 1502-1505), using 4-morpholinoaniline as a starting material, and obtained as a hydrochloride.

ESI/MS m/e: 250.1 (M⁺+H, C₁₄H₂₃N₃O).

### Reference Example 10

### Synthesis of ((2S)-2-aminobutyl)(4-piperidin-1-ylphenyl)amine (Reference Example Compound 10)

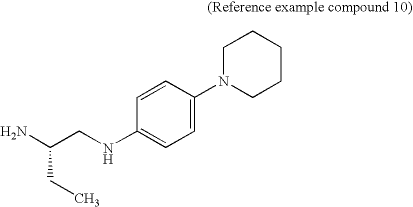

### Reference Example Compound 10

The reference example compound 10 was synthesized according to the method described in the literature (Bioorg. Med. Chem. Lett., 2006, 16, 1502-1505), using 4-piperidin-1-ylaniline as a starting material, and obtained as a hydrochloride.

ESI/MS m/e: 248.2 (M+H, C₁₅H₂₅N₃).

### Example 1

### Synthesis of N-((1S)-1-{[(2,4-dimethoxyphenyl)amino]methyl}propyl){[(2,2,2-trifluoro-1-phenylethyl)amino]cyclohexyl}carboxamide (8) (Route A)

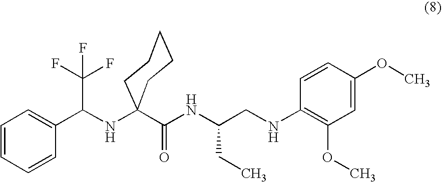

1-[(2,2,2-Trifluoro-1-phenylethyl)amino]cyclohexane carboxylic acid (Reference Example Compound 3: 15 mg) was dissolved in N,N-dimethylformamide (500 μL). To this solution, HATU (19 mg) and triethylamine (7 μL) were added under ice cooling, and the solution was stirred. This solution was added to ((2S)-2-aminobutyl)(2,4-dimethoxyphenyl)amine (Reference Example Compound 7: 18 mg, hydrochloride) under ice cooling, and further triethylamine (14 μL) was added to the mixture solution. The mixture was stirred for 1 hour under ice cooling. The reaction was quenched with saturated aqueous ammonium chloride solution. The organic layer was extracted with ethyl acetate, washed with saturated saline, dried over anhydrous sodium sulfate, and filtered. The filtrate was concentrated in vacuo and the residue was purified by high performance liquid chromatography to obtain the title compound (8: 19 mg, trifluoroacetate).

In addition, a portion of the obtained title compound (8, trifluoroacetate) was dissolved in ethyl acetate and the solution was washed with aqueous sodium hydrogen carbonate solution. The organic layer was dried over anhydrous sodium sulfate and filtered. The filtrate was concentrated in vacuo to obtain the title compound (8, free base).

¹H-NMR (400 MHz, CDCl₃) δ (ppm): 7.37-7.27 (m, 5H), 6.96 (t, J=7.7 Hz, 1H), 6.61 (d, J=8.5 Hz, 0.5H), 6.55 (d, J=8.5 Hz, 0.5H), 6.47-6.39 (m, 2H), 4.15-3.98 (m, 2H), 3.82 (s, 1.5H), 3.79 (s, 1.5H), 3.76 (s, 1.5H), 3.75 (s, 1.5H), 3.25-2.94 (m, 2H), 2.21-2.09 (m, 1H), 2.06-1.94 (m, 1H), 1.86-1.75 (m, 1H), 1.72-1.18 (m, 8H), 1.00-0.85 (m, 4H).

ESI/MS m/e: 508.2 (M⁺+H, C₂₇H₃₆F₃N₃O₃).

### Example 2

### Synthesis of (2S)—N-((1S)-1-{[(4-methoxyphenyl)amino]methyl}propyl)-2-[((1S)-2,2,2-trifluoro-1-{4-[4-(methylsulfonyl)phenyl]phenyl}ethyl)amino]-4-fluoro-4-methylpentanamide (1) (Route A)

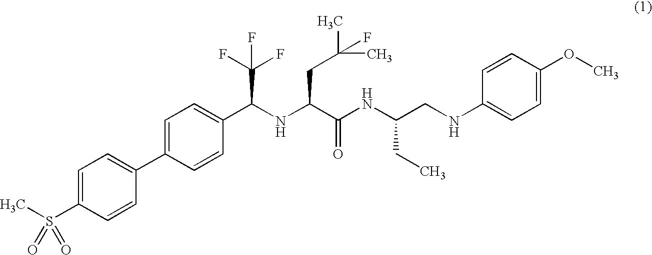

Similarly to Example 1, by reacting (2S)-2-[((1S)-2,2,2-trifluoro-1-{4-[4-(methylsulfonyl)phenyl]phenyl}ethyl)amino]-4-fluoro-4-methylpentanoic acid (Reference Example Compound 1: 20 mg) with ((2S)-2-aminobutyl)(4-methoxyphenyl)amine (Reference Example Compound 4: 17 mg), the title compound (1: 24 mg, trifluoroacetate) was obtained.

¹H-NMR (400 MHz, CDCl₃) δ (ppm): 7.99 (d, J=8.5 Hz, 2H), 7.64 (d, J=8.5 Hz, 2H), 7.42 (d, J=8.3 Hz, 2H), 7.34 (d, J=8.0 Hz, 2H), 6.96 (d, J=9.3 Hz, 1H), 6.73 (d, J=9.0 Hz, 2H), 6.49 (d, J=8.8 Hz, 2H), 4.17 (t, J=7.1 Hz, 1H), 4.07-3.95 (m, 1H), 3.76-3.71 (m, 4H), 3.09 (s, 3H), 3.05-2.99 (m, 2H), 2.78-2.72 (m, 1H), 2.18-1.92 (m, 2H), 1.65-1.30 (m, 8H), 0.88 (t, J=7.4 Hz, 3H).

ESI/MS m/e: 638.2 (M⁺+H, C₃₂H₃₉F₄N₃O₄S).

### Example 3

### Synthesis of N-{(1R)-2-[(4-methoxyphenyl)amino]-1-[(phenylmethoxy)methyl]ethyl}(2S)-2-[((1S)-2,2,2-trifluoro-1-{4-[4-(methylsulfonyl)phenyl]phenyl}ethyl)amino]-4-fluoro-4-methylpentanamide (2) (Route A)

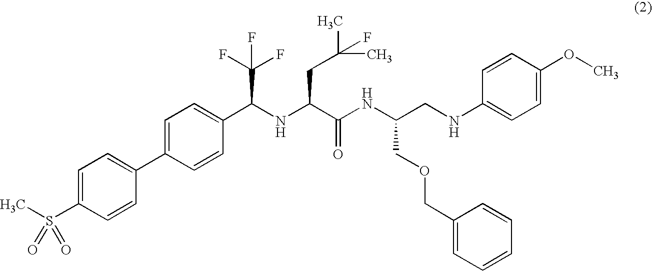

Similarly to Example 1, by reacting (2S)-2-[((1S)-2,2,2-trifluoro-1-{4-[4-(methylsulfonyl)phenyl]phenyl}ethyl)amino]-4-fluoro-4-methylpentanoic acid (Reference Example Compound 1: 20 mg) with ((2S)-2-amino-3-benzyloxypropyl)(4-methoxyphenyl)amine (Reference Example Compound 5: 19 mg), the title compound (2: 22 mg, trifluoroacetate) was obtained.

¹H-NMR (400 MHz, CDCl₃) δ (ppm): 7.97 (d, J=8.3 Hz, 2H), 7.63-7.53 (m, 3H), 7.41-7.26 (m, 9H), 6.69 (d, J=9.0 Hz, 2H), 6.48 (d, J=9.0 Hz, 2H), 4.48 (d, J=12.2 Hz, 1H), 4.44 (d, J=12.2 Hz, 1H), 4.22-4.15 (m, 2H), 3.72 (s, 3H), 3.67-3.56 (m, 2H), 3.46 (dd, J=9.5 Hz, J=3.9 Hz, 1H), 3.10 (s, 3H), 3.08-2.99 (m, 2H), 2.93 (brs, 1H), 2.17-1.88 (m, 2H), 1.53-1.40 (m, 6H).

ESI/MS m/e: 730.2 (M⁺+H, C₃₈H₄₃F₄N₃O₅S).

### Example 4

### Synthesis of N-((1R)-2-hydroxy-1-{[(4-methoxyphenyl)amino]methyl}ethyl)(2S)-2-[((1S)-2,2,2-trifluoro-1-{4-[4-(methylsulfonyl)phenyl]phenyl}ethyl)amino]-4-fluoro-4-methylpentanamide (3) (Route A)

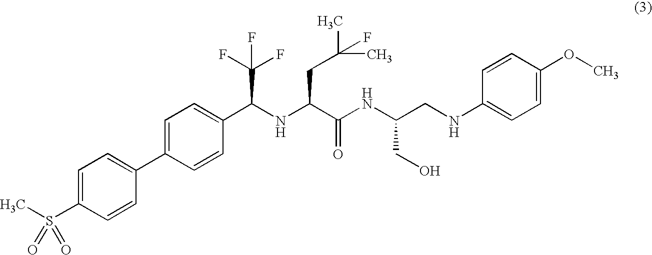

Similarly to Example 1, by reacting (2S)-2-[((1S)-2,2,2-trifluoro-1-{4-[4-(methylsulfonyl)phenyl]phenyl}ethyl)amino]-4-fluoro-4-methylpentanoic acid (Reference Example Compound 1: 33 mg) with {(2S)-2-amino-3-(tert-butyldimethylsiloxy)propyl}(4-methoxyphenyl)amine (Reference Example Compound 6: 27 mg), N-{(1R)-2-[(4-methoxyphenyl)amino]-1-[(1,1,2,2-tetramethyl-1-silapropoxy)methyl]ethyl}(2S)-2-[((1S)-2,2,2-trifluoro-1-{4-[4-(methylsulfonyl)phenyl]phenyl}ethyl)amino]-4-fluoro-4-methylpentanamide (48 mg, free base) was obtained.

The N-{(1R)-2-[(4-methoxyphenyl)amino]-1-[(1,1,2,2-tetramethyl-1-silapropoxy)methyl]ethyl}(2S)-2-[((1S)-2,2,2-trifluoro-1-{4-[4-(methylsulfonyl) phenyl]phenyl}ethyl)amino]-4-fluoro-4-methylpentanamide was dissolved in methanol (0.64 mL) and then hydrogen chloride (64 μL, 4 mol/L dioxane solution) was added to the mixture solution. The mixture was stirred at room temperature for 1.5 hours. This reaction solution was concentrated in vacuo and the residue was purified by high performance liquid chromatography (neutral system). To a fraction containing the title compound (3), 6 mol/L hydrochloric acid (20 mL) was added, and the mixture solution was concentrated in vacuo to obtain the title compound (3: 32 mg, hydrochloride).

¹H-NMR (400 MHz, CDCl₃) δ (ppm): 7.99 (d, J=8.5 Hz, 2H), 7.65 (d, J=8.5 Hz, 2H), 7.55 (d, J=7.8 Hz, 1H), 7.45-7.38 (m, 4H), 6.71 (d, J=8.8 Hz, 2H), 6.53 (d, J=8.8 Hz, 2H), 4.27-4.20 (m, 1H), 4.10-4.00 (m, 1H), 3.76-3.62 (m, 6H), 3.17-3.10 (m, 4H), 3.07-2.92 (m, 2H), 2.20-1.95 (m, 2H), 1.50 (d, J=11.0, 3H), 1.45 (d, J=11.0, 3H).

ESI/MS m/e: 640.2 (M⁺+H, C₃₁H₃₇F₄N₃O₅S).

### Example 5

### Synthesis of (2S)—N-((1S)-1-{[(3,4-diethoxyphenyl)amino]methyl}propyl)-2-[((1S)-2,2,2-trifluoro-1-{4-[4-(methylsulfonyl)phenyl]phenyl}ethyl)amino]-4-fluoro-4-methylpentanamide (4) (Route A)

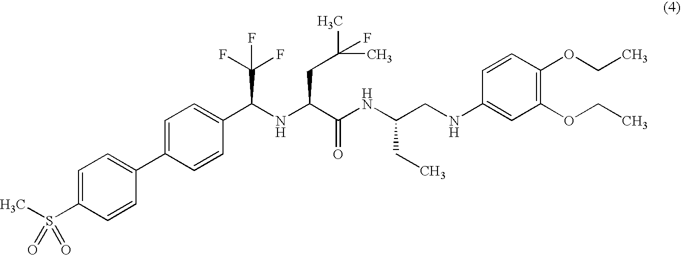

Similarly to Example 1, by reacting (2S)-2-[((1S)-2,2,2-trifluoro-1-{4-[4-(methylsulfonyl)phenyl]phenyl}ethyl)amino]-4-fluoro-4-methylpentanoic acid (Reference Example Compound 1: 23 mg) with ((2S)-2-aminobutyl)(3,4-diethoxyphenyl)amine (Reference Example Compound 8: 20 mg, hydrochloride), the title compound (4: 23 mg, trifluoroacetate) was obtained.

¹H-NMR (400 MHz, CDCl₃) δ (ppm): 7.99 (d, J=8.5 Hz, 2H), 7.65 (d, J=8.5 Hz, 2H), 7.42 (d, J=8.3 Hz, 2H), 7.32 (d, J=8.0 Hz, 2H), 6.96 (d, J=9.5 Hz, 1H), 6.75 (d, J=8.5 Hz, 1H), 6.14 (d, J=2.7 Hz, 1H), 6.03 (dd, J=8.5 Hz, 2.7 Hz, 1H), 4.23-4.14 (m, 1H), 4.07-3.90 (m, 5H), 3.74 (d, J=9.5 Hz, 1H), 3.66 (brs, 1H), 3.12 (s, 3H), 3.03 (dd, J=11.6 Hz, J=3.8 Hz, 2H), 2.72 (dd, J=11.6 Hz, J=8.9 Hz, 1H), 2.18-1.92 (m, 2H), 1.63-1.55 (m, 1H), 1.51 (d, J=17.7 Hz, 3H), 1.46 (d, J=17.7 Hz, 3H), 1.42-1.32 (m, 7H), 0.89 (t, J=7.4 Hz, 3H).

ESI/MS m/e: 696.3 (M⁺+H, C₃₅H₄₅F₄N₃O₅S).

### Example 6

### Synthesis of (2S)—N-((1S)-1-{[(2,4-dimethoxyphenyl)amino]methyl}propyl)-2-[((1S)-2,2,2-trifluoro-1-{4-[4-(methylsulfonyl)phenyl]phenyl}ethyl)amino]-4-fluoro-4-methylpentanamide (5) (Route A)

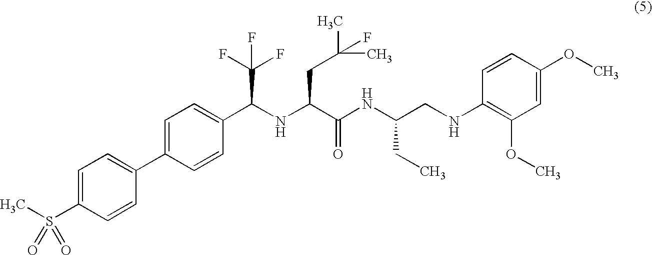

Similarly to Example 1, by reacting (2S)-2-[((1S)-2,2,2-trifluoro-1-{4-[4-(methylsulfonyl)phenyl]phenyl}ethyl)amino]-4-fluoro-4-methylpentanoic acid (Reference Example Compound 1: 23 mg) with ((2S)-2-aminobutyl)(2,4-dimethoxyphenyl)amine (Reference Example Compound 7: 18 mg, hydrochloride), the title compound (5: 28 mg, trifluoroacetate) was obtained.

¹H-NMR (400 MHz, CDCl₃) δ (ppm): 7.99 (d, J=8.5 Hz, 2H), 7.66 (d, J=8.5 Hz, 2H), 7.38 (d, J=8.3 Hz, 2H), 7.29 (d, J=8.0 Hz, 2H), 6.88 (d, J=9.3 Hz, 1H), 6.47-6.35 (m, 3H) 4.26-4.14 (m, 1H), 4.10-4.00 (m, 1H), 3.83-3.72 (m, 7H), 3.13-3.02 (m, 5H), 2.73 (dd, J=12.1 Hz, J=4.6 Hz, 1H), 2.19-1.92 (m, 2H), 1.63-1.31 (m, 8H), 0.88 (t, J=7.4 Hz, 3H).

ESI/MS m/e: 668.2 (M⁺+H, C₃₃H₄₁F₄N₃O₅S).

### Example 7

### Synthesis of (2S)—N-((1S)-1-{[(2,4-dimethoxyphenyl)amino]methyl}propyl)-2-{[(1S)-1-(4-bromophenyl)-2,2,2-trifluoroethyl]amino}-4-fluoro-4-methylpentanamide(6) (Route A)

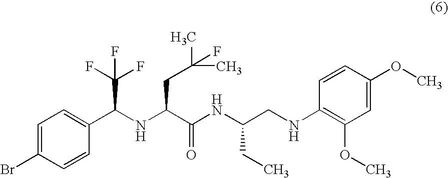

Similarly to Example 1, by reacting (2S)-2-[{(1S)-2,2,2-trifluoro-1-(4-bromophenyl)ethyl}amino]-4-fluoro-4-methylpentanoic acid (Reference Example Compound 2: 50 mg) with ((2S)-2-aminobutyl)(2,4-dimethoxyphenyl)amine (Reference Example Compound 7: 46 mg, hydrochloride), the title compound (6: 22 mg, trifluoroacetate) was obtained.

¹H-NMR (400 MHz, CDCl₃) δ (ppm): 7.29-7.26 (2H, m), 7.00 (2H, d, J=8.0 Hz), 6.74 (1H, d, J=9.3 Hz), 6.49-6.45 (3H, m), 4.11-4.01 (2H, m), 3.83 (3H, d, J=1.0 Hz), 3.78 (3H, d, J=1.2 Hz), 3.08-3.00 (2H, m), 2.73-2.68 (1H, m), 2.15-1.90 (2H, m), 1.61-1.55 (2H, m), 1.52-1.42 (6H, m), 0.88 (3H, t, J=7.3 Hz).

ESI/MS m/e: 593.1 (M⁺+H, C₂₆H₃₄BrF₄N₃O₃).

### Example 8

### Synthesis of (2S)—N-((1S)-1-{[(4-morpholin-4-ylphenyl)amino]methyl}propyl)-2-[((1S)-2,2,2-trifluoro-1-{4-[4-(methylsulfonyl)phenyl]phenyl}ethyl)amino]-4-fluoro-4-methylpentanamide(9) (Route A)

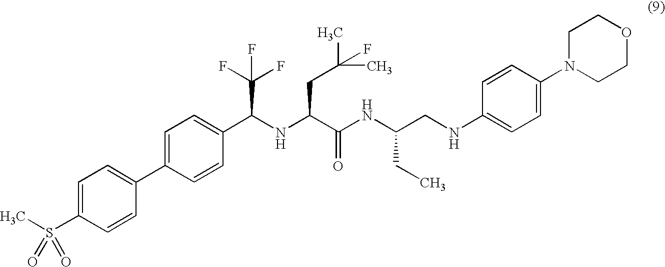

Similarly to Example 1, by reacting (2S)-2-[((1S)-2,2,2-trifluoro-1-{4-[4-(methylsulfonyl)phenyl]phenyl}ethyl)amino]-4-fluoro-4-methylpentanoic acid (Reference Example Compound 1: 20 mg) with ((2S)-2-aminobutyl)(4-morpholin-4-ylphenyl)amine (Reference Example Compound 9: 22 mg, hydrochloride), the title compound (9: 8 mg, trifluoroacetate) was obtained.

ESI/MS m/e: 693.2 (M⁺+H, C₃₅H₄₄F₄N₄O₄S).

### Example 9

### Synthesis of (2S)—N-((1S)-1-{[(4-piperidin-1-ylphenyl)amino]methyl}propyl)-2-[((1S)-2,2,2-trifluoro-1-{4-[4-(methylsulfonyl)phenyl]phenyl}ethyl)amino]-4-fluoro-4-methylpentanamide(10) (Route A)

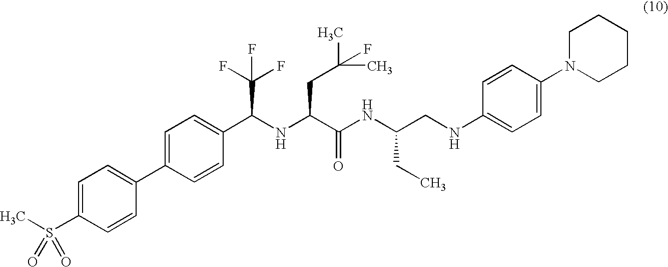

Similarly to Example 1, by reacting (2S)-2-[((1S)-2,2,2-trifluoro-1-{4-[4-(methylsulfonyl)phenyl]phenyl}ethyl)amino]-4-fluoro-4-methylpentanoic acid (Reference Example Compound 1: 20 mg) with ((2S)-2-aminobutyl)(4-piperidin-1-ylphenyl)amine (Reference Example Compound 10: 22 mg, hydrochloride), the title compound (10: 18 mg, trifluoroacetate) was obtained.

ESI/MS m/e: 691.2 (M⁺+H, C₃₆H₄₆F₄N₄O₃S).

### Example 10

### Synthesis of N-((1S)-1-{[(4-morpholin-1-ylphenyl)amino]methyl}propyl) {[(2,2,2-trifluoro-1-phenylethyl)amino]cyclohexyl}carboxamide(11) (Route A)

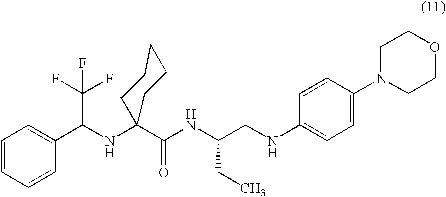

Similarly to Example 1, by reacting 1-[(2,2,2-trifluoro-1-phenylethyl)amino]cyclohexanecarboxylic acid (Reference Example Compound 3: 9 mg) with ((2S)-2-aminobutyl)(4-morpholin-4-ylphenyl)amine (Reference Example Compound 9: 12 mg, hydrochloride), the title compound (11: 8 mg, trifluoroacetate) was obtained.

ESI/MS m/e: 533.3 (M⁺+H, C₂₉H₃₉F₃N₄O₂).

### Example 11

### Synthesis of (2S)—N-((1S)-1-{[(2,4-dimethoxyphenyl)amino]methyl}propyl)-2-[((1S)-2,2,2-trifluoro-1-phenylethyl)amino]-4-fluoro-4-methylpentanamide(7) (Route G)

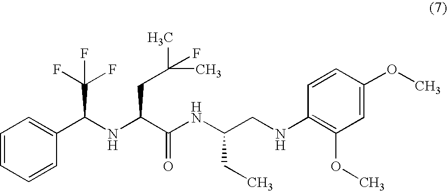

(2S)—N-((1S)-1-{[(2,4-dimethoxyphenyl)amino]methyl}propyl)-2-{[(1S)-1-(4-bromophenyl)-2,2,2-trifluoroethyl]amino}-4-fluoro-4-methylpentanamide (6: 31 mg) was dissolved in methanol (1 mL). To this solution, palladium-activated carbon (10% Pd) (3 mg) was added, and the mixture was stirred under hydrogen atmosphere at room temperature for 2.5 hours. The reaction solution was filtered through celite and celite was washed with ethyl acetate. The filtrate was concentrated in vacuo and the residue was purified by high performance liquid chromatography to obtain the title compound (7: 13 mg, trifluoroacetate).

¹H-NMR (400 MHz, CDCl₃) δ (ppm): 7.30-7.26 (1H, m), 7.24-7.18 (4H, m), 6.78 (1H, d, J=9.5 Hz), 6.48-6.41 (3H, m), 4.13-3.95 (2H, m), 3.85-3.81 (3H, m), 3.81-3.77 (3H, m), 3.12-3.04 (1H, m), 2.99 (1H, dd, J=12.3, 5.0 Hz), 2.70 (1H, dd, J=12.2, 7.8 Hz), 2.16-1.91 (2H, m), 1.60-1.39 (8H, m), 0.85 (3H, t, J=7.4 Hz).

ESI/MS m/e: 514.3 (M⁺+H, C₂₆H₃₅F₄N₃O₃).

### Reference Example 11

### Synthesis of {(1S)-1-[(1,1-diethyl-1-silapropoxy)methyl]-3-methylbutyl}[(1S)-2,2,2-trifluoro-1-(4-methylthiophenyl)ethyl]amine (Reference Example Compound 11)

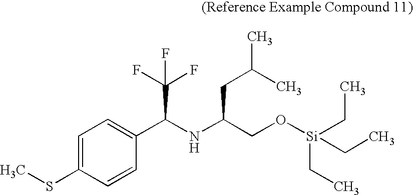

### Reference Example Compound 11

Reference example compound 11 was synthesized according to the method described in the literature (WO2003/075836 and J. Org. Chem., 2006, 71, 4320-4323), using 1-bromo-4-methylthiobenzene as a starting material.

ESI/MS m/e: 436.2 (M⁺+H, C₂₁H₃₆F₃NOSSi).

### Reference Example 12

### Synthesis of (1-(2H-benzo[3,4-d]1,3-dioxolen-5-yl)(1S)-2,2,2-trifluoroethyl){(1S)-1-[(1,1-diethyl-1-silapropoxy)methyl]-3-methylbutyl}amine (Reference Example Compound 12)

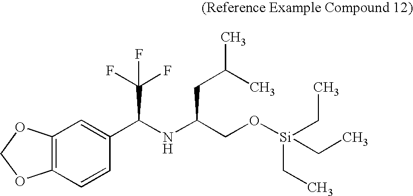

### Reference Example Compound 12

Reference example compound 12 was synthesized according to the method described in Reference Example A, using 4-bromo-1,2-(methylenedioxy)benzene as a starting material.

ESI/MS m/e: 434.2 (M⁺+H, C₂₁H₃₄F₃NO₃Si).

### Reference Example 13

### Synthesis of 2,2,2-trifluoro-1-[4-(1,1,2,2-tetramethyl-1-silapropoxy)phenyl]ethan-1-one (Reference Example Compound 13)

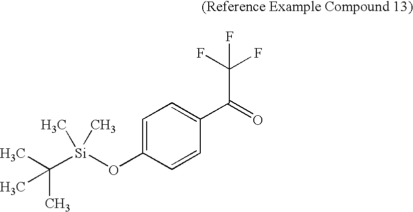

### Reference Example Compound 13

Reference example compound 13 was synthesized according to the method described in the literature (J. Org. Chem., 1991, 56, 2, 893-896), using 1-bromo-4-(1,1,2,2-tetramethyl-1-silapropoxy)benzene as a starting material.

ESI/MS m/e: 247.2 (M⁺+H, C₁₄H₁₉F₃O₂Si).

Hereinafter, the compounds described in Reference Example 14 to Reference Example 18 were synthesized according to the method described in Reference Example 13, using the corresponding starting materials and reagents. Their structures and M⁺+H observed by GC/MS, i.e., the measured value observed as the value of the compound molecular weight (M) with proton (H⁺), are summarized in Table 2 below.

TABLE 2

| Reference | | |
|---|---|---|
| Example | | |
| No. | Structure | M⁺ + H |
| 14 | 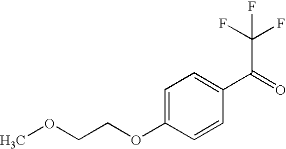 | 248.1 |
| 15 | 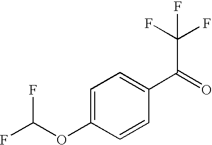 | 240.1 |
| 16 | 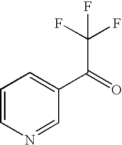 | 175.1 |
| 17 | 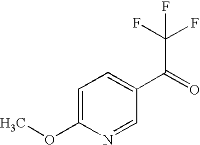 | 205.1 |
| 18 | 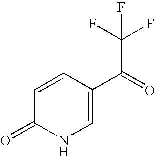 | 191.1 |

Hereinafter, the compounds described in Reference Example 19 to Reference Example 44 were synthesized according to the method described in Reference Examples 1 to 3, using the corresponding starting materials and reagents. Their structures, NMR spectra and M⁺+H observed by LC/MS, i.e., the measured value observed as the value of the compound molecular weight (M) with proton (H⁺), are summarized in Table 3 below.

TABLE 3

| Reference | | | |
|---|---|---|---|
| Example No. | Structure | M⁺ + H | NMR |
| 19 | 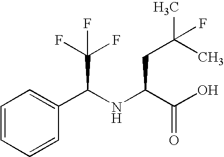 | 308.1 | ¹H-NMR (CDCl₃) δ: 7.43-7.36 (5H, m), 4.21 (1H, q, J = 7.2 Hz), 3.64 (1H, dd, J = 7.9, 4.3 Hz), 2.23-2.10 (1H, m), 2.03-1.90 (1H, m), 1.48 (3H, d, J = 8.3 Hz), 1.43 (3H, d, J = 8.3 Hz). |
| 20 | 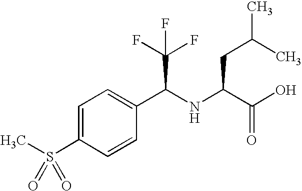 | 368.1 | |
| 21 | 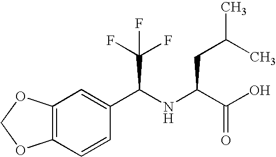 | 334.1 | |
| 22 | 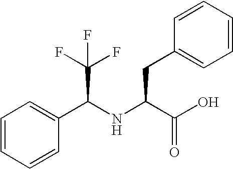 | 324.1 | ¹H-NMR (CDCl₃) δ: 7.40-7.23 (10H, m), 3.97 (1H, q, J = 7.2 Hz), 3.62 (1H, t, J = 6.1 Hz), 3.12 (2H, ddd, J = 45.3, 13.8, 6.2 Hz). |
| 23 | 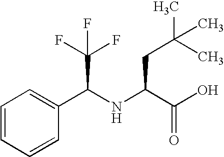 | 304.2 | ¹H-NMR (CDCl₃) δ: 7.41-7.36 (5H, m), 4.09 (1H, q, J = 7.1 Hz), 3.61 (1H, dd, J = 8.0, 3.9 Hz), 1.72 (1H, dd, J = 14.1, 3.9 Hz), 1.44 (1H, dd, J = 14.3, 7.9 Hz), 1.02 (9H, s). |
| 24 | 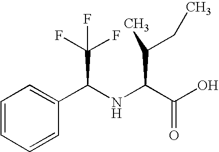 | 290.1 | ¹H-NMR (CDCl₃) δ: 7.38 (5H, s), 4.09 (1H, q, J = 7.2 Hz), 3.36 (1H, d, J = 4.9 Hz), 1.82 (1H, ddt, J = 16.9, 10.0, 4.1 Hz), 1.57-1.46 (1H, m), 1.34-1.21 (1H, m), 1.01 (3H, d, J = 6.8 Hz), 0.91 (3H, t, J = 7.3 Hz). |
| 25 | 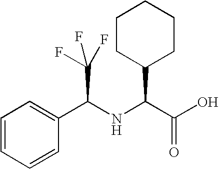 | 316.1 | ¹H-NMR (CDCl₃) δ: 7.38 (5H, s), 4.08 (1H, q, J = 7.2 Hz), 3.30 (1H, d, J = 5.1 Hz), 1.79-1.67 (6H, m), 1.35-1.09 (5H, m). |
| 26 | 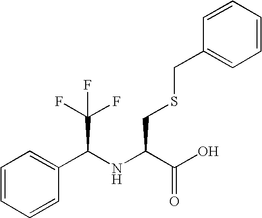 | 370 | ¹H-NMR (CDCl₃) δ: 7.45-7.16 (10H, m), 4.11 (1H, q, J = 7.0 Hz), 3.75 (2H, s), 3.52 (1H, t, J = 5.5 Hz), 2.85 (2H, ddd, J = 29.9, 14.0, 5.5 Hz). |
| 27 | 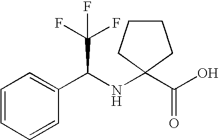 | 288.1 | ¹H-NMR (CDCl₃) δ: 7.40-7.34 (5H, m), 4.12 (1H, q, J = 7.6 Hz), 2.14- 2.01 (2H, m), 1.82-1.57 (5H, m), 1.49-1.43 (1H, m). |
| 28 | 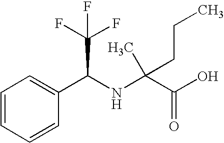 | 290.1 | ¹H-NMR (CDCl₃) δ: 7.41-7.33 (5H, m), 4.21 (1H, q, J = 7.7 Hz), 1.70- 1.61 (1H, m), 1.58-1.50 (1H, m), 1.33-1.21 (3H, m), 1.19 (2H, s), 0.85 (3H, t, J = 7.3 Hz). |
| 29 | 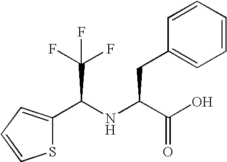 | 330 | ¹H-NMR (CDCl₃) δ: 7.41-7.22 (6H, m), 7.07-6.99 (2H, m), 4.28 (1H, q, J = 6.7 Hz), 3.69 (1H, t, J = 6.2 Hz), 3.11 (2H, ddd, J = 47.1, 14.0, 6.2 Hz). |
| 30 | 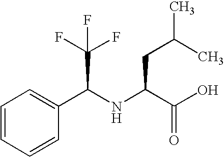 | 290.1 | ¹H-NMR (CDCl₃) δ: 7.40-7.36 (5H, m), 4.12 (1H, q, J = 7.2 Hz), 3.53 (1H, dd, J = 8.5, 5.6 Hz), 1.95- 1.85 (1H, m), 1.62-1.46 (2H, m), 0.95 (6H, t, J = 6.0 Hz). |
| 31 | 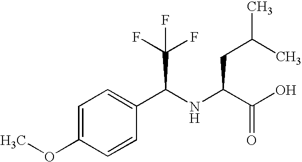 | 320.1 | ¹H-NMR (CDCl₃) δ: 7.32 (2H, d, J = 8.3 Hz), 6.90 (2H, d, J = 8.8 Hz), 4.13 (1H, q, J = 7.1 Hz), 3.81 (3H, s), 3.52 (1H, dd, J = 8.3, 5.6 Hz), 1.93-1.83 (1H, m), 1.64-1.47 (2H, m), 0.94 (6H, t, J = 5.9 Hz). |
| 32 | 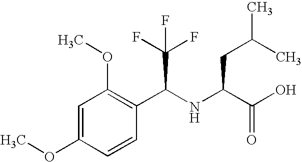 | 350.1 | |
| 33 | 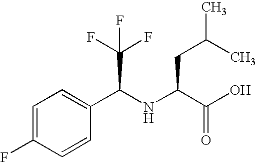 | 308.2 | |
| 34 | 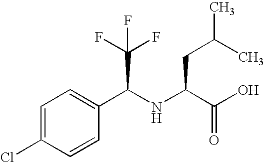 | 324.1 | |
| 35 | 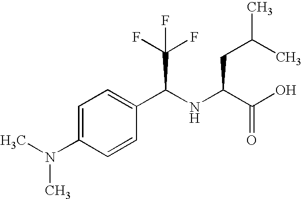 | 333.2 | |
| 36 | 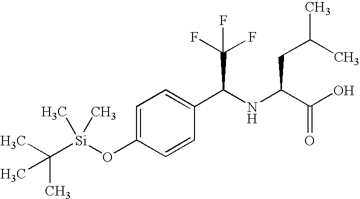 | 420.2 | |
| 37 | 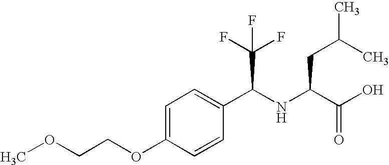 | 364.2 | |
| 38 | 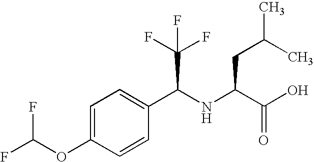 | 356.1 | |
| 39 | 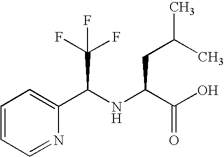 | 291.1 | |
| 40 | 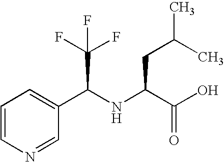 | 291.1 | |
| 41 | 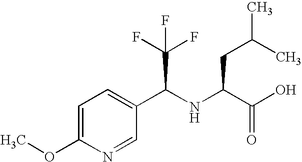 | 321.1 | |
| 42 | 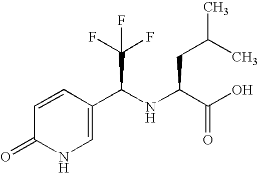 | 307.1 | |
| 43 | 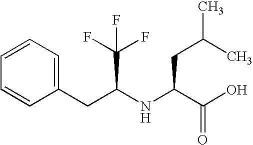 | 304.2 | |
| 44 | 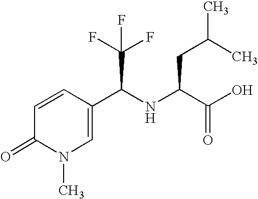 | 321.2 | |

### Reference Example 45

### Synthesis of 1-(4-nitrophenyl)pyrrolidin-2-one (Reference Example Compound 45)

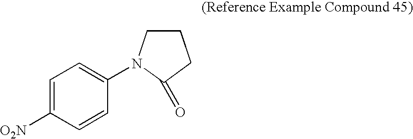

### Reference Example Compound 45

Reference example compound 45 was synthesized according to the method described in the literature (Tetrahedron, 1988, 44, 10, 3025-3036), using 4-nitroaniline as a starting material.

ESI/MS m/e: 207.1 (M⁺+H, C₁₀H₁₀N₂O₃).

### Reference Example 46

### Synthesis of ethyl 1-(4-nitrophenyl)piperidine-4-carboxylate (Reference Example Compound 46)

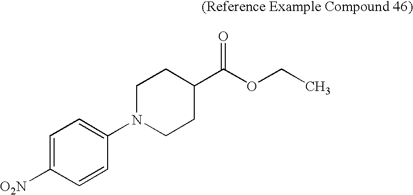

### Reference Example Compound 46

Reference example compound 46 was synthesized according to the method described in the literature (WO2005/058824), using 1-fluoro-4-nitrobenzene and ethyl isonipecotate as a starting material.

ESI/MS m/e: 279.2 (M⁺+H, C₁₄H₁₈N₂O₄).

Hereinafter, the compounds described in Reference Example 47 to Reference Example 49 were synthesized according to the method described in Reference Example 46, using the corresponding starting materials and reagents. Their structure and M⁺+H observed by LC/MS, i.e., the measured value observed as the value of the compound molecular weight (M) with proton (HR) added are summarized in Table 4 below.

TABLE 4

| Reference | | |
|---|---|---|
| Example No. | Structure | M⁺ + H |
| 47 | 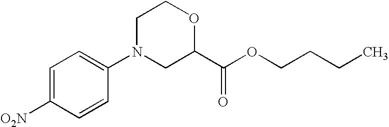 | 309.1 |
| 48 | 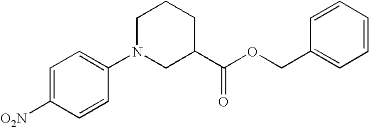 | 341.1 |
| 49 | 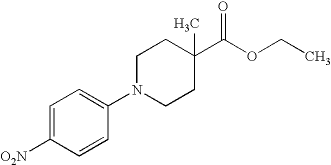 | 293.2 |

### Reference Example 50

### Synthesis of phenylmethyl 1-(3-methoxy-4-nitrophenyl)cyclopropanecarboxylate (Reference Example Compound 50)

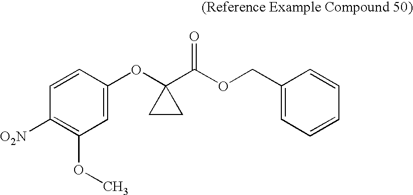

### Reference Example Compound 50

Sodium hydride (50 to 72% in mineral oil, 92 mg) was suspended in tetrahydrofuran (2.7 mL). To this suspension, a tetrahydrofuran solution (2.0 mL) of benzyl 1-hydroxy-1-cyclopropanecarboxylate (404 mg) was added dropwise under ice-cooling and the mixture was stirred at room temperature for 10 minutes. After adding 18-crown-6-ether (26 mg) under ice-cooling to the reaction solution, 1-fluoro-3-methoxy-4-nitrobenzene (342 mg) was added in small portions and the mixture was stirred at room temperature for 42 hours. The reaction was quenched with a 1:1 mixed solution of saturated aqueous ammonium chloride solution and saturated saline, and extracted with ethyl acetate The organic layer was washed with saturated saline, dried over anhydrous sodium sulfate, and filtered. The filtrate was concentrated in vacuo and the residue was purified by silica gel column chromatography to obtain the title compound (50: 524 mg).

ESI/MS m/e: 344.2 (M⁺+H, C₁₈H₁₇NO₆).

Hereinafter, the compounds described in Reference Example 51 to Reference Example 57 were synthesized by converting the nitro of the corresponding starting materials into amino, through hydrogen reduction in the presence of Pd catalyst (Reference literature: J. Med. Chem., 2000, 43, 3052-3066) or reduction using a reducing agent such as tin (II) chloride iron (Reference literature: Synthesis, 1999, 7, 1246-1250, Bioorg. Med. Chem., 2007, 15, 5912-5949, etc.) and the like, according to the common reduction method of nitro. Their structures, NMR spectra and M⁺+H observed by LC/MS, i.e., the measured value observed as the value of the compound molecular weight (M) with proton (H⁺) are summarized in Table 5 below.

TABLE 5

| Reference | | |
|---|---|---|
| Example No. | Structure | M⁺ + H |
| 51 | 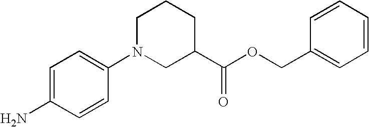 | 311.2 |
| 52 | 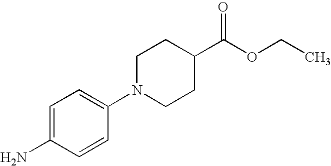 | 249.2 |
| 53 | 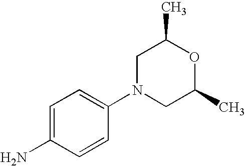 | 207.2 |
| 54 | 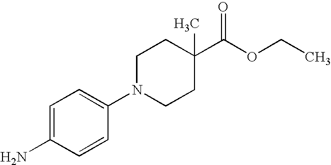 | 263.2 |
| 55 | 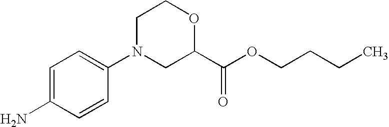 | 279.1 |
| 56 | 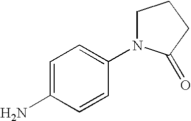 | 177.1 |
| 57 | 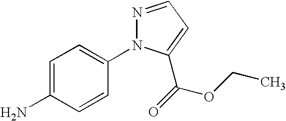 | 232.1 |

Hereinafter, the compounds described in Reference Example 58 to Reference Example 97 were synthesized according to the method described in the literature (Bioorg. Med. Chem., 2006, 14, 6789-6806), using the corresponding starting materials and reagents, similarly to Reference Examples 4-10. Their structures and M⁺+H observed by LC/MS, i.e., the measured value observed as the value of the compound molecular weight (M) with proton (H⁺) are summarized in Table 6 below.

TABLE 6

| Reference | | |
|---|---|---|
| Example No. | Structure | M⁺ + H |
| 58 | 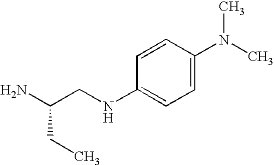 | 208.1 |
| 59 | 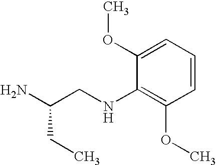 | 224.3 |
| 60 | 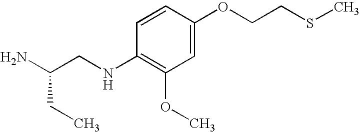 | 285.1 |
| 61 | 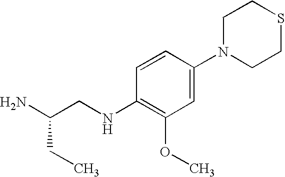 | 296.1 |
| 62 | 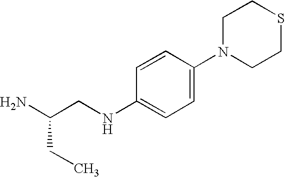 | 266.1 |
| 63 | 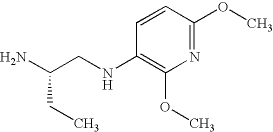 | 226.1 |
| 64 | 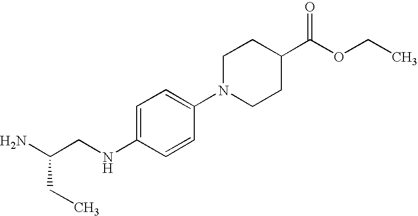 | 320.2 |
| 65 | 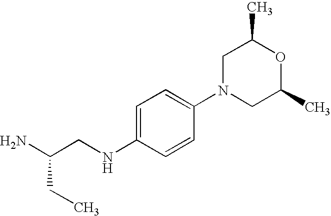 | 278.2 |
| 66 | 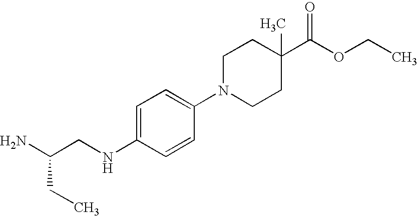 | 334.3 |
| 67 | 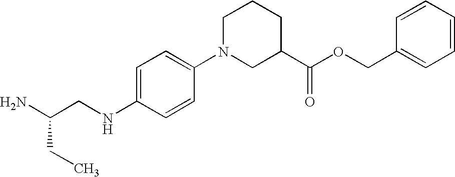 | 382.2 |
| 68 | 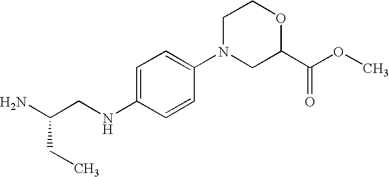 | 308.2 |
| 69 | 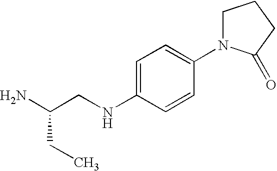 | 248.2 |
| 70 | 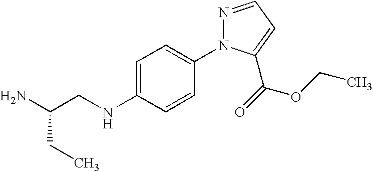 | 231.2 |
| 71 | 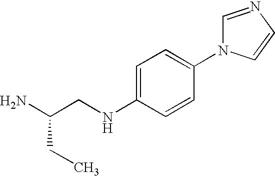 | 231.2 |
| 72 | 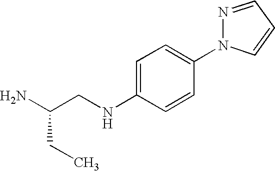 | 303.2 |
| 73 | 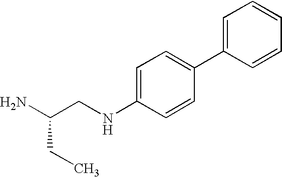 | 241.2 |
| 74 | 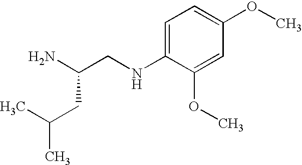 | 253.1 |
| 75 | 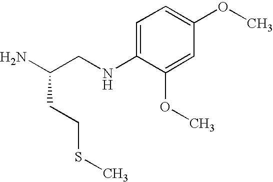 | 271.1 |
| 76 | 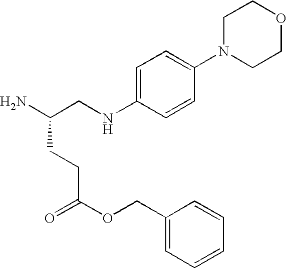 | 384.2 |
| 77 | 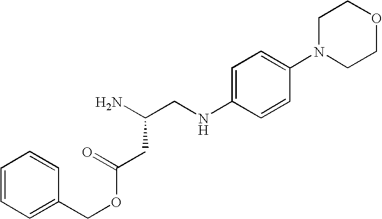 | 370.2 |
| 78 | 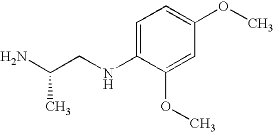 | 211.1 |
| 79 | 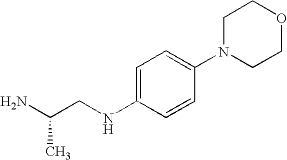 | 236.1 |
| 80 | 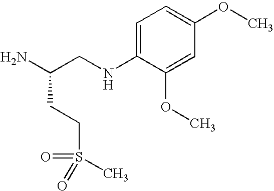 | 303.1 |
| 81 | 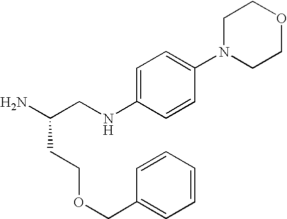 | 356.2 |
| 82 | 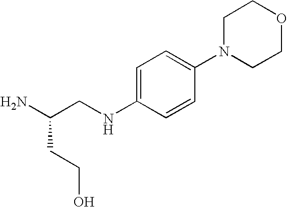 | 266.1 |
| 83 | 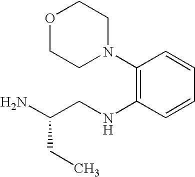 | 250.2 |
| 84 | 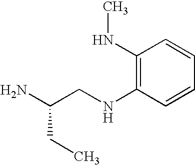 | 194.2 |
| 85 | 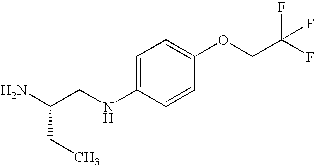 | 263.1 |
| 86 | 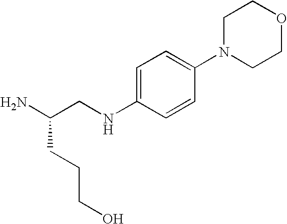 | 380.2 |
| 87 | 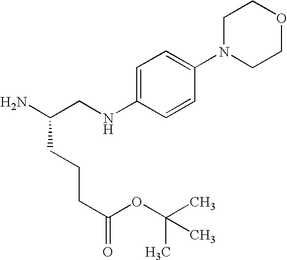 | 364.3 |
| 88 | 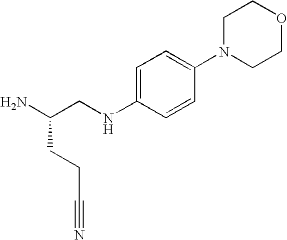 | 275.1 |
| 89 | 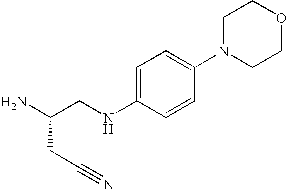 | 261.1 |
| 90 | 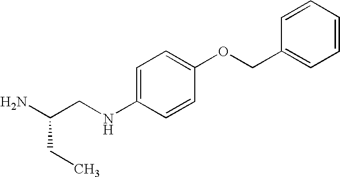 | 271.1 |
| 91 | 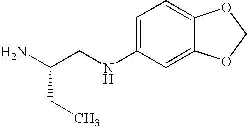 | 209.1 |
| 92 | 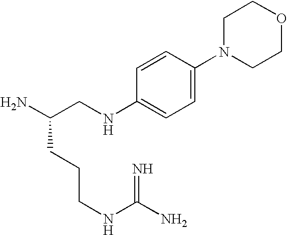 | 321.2 |
| 93 | 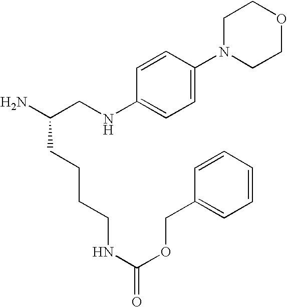 | 427.2 |
| 94 | 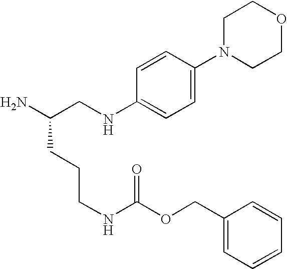 | 413.2 |
| 95 | 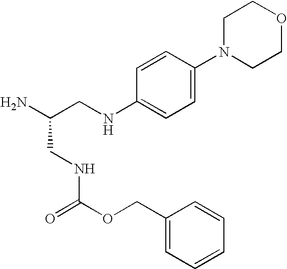 | 385.2 |
| 96 | 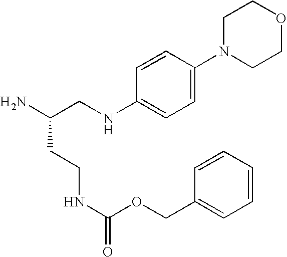 | 399.2 |
| 97 | 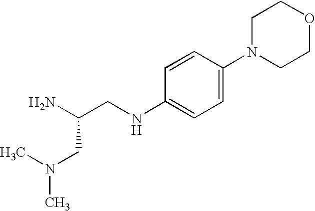 | 279.1 |

### Reference Example 98

### Synthesis of N-((1S)-1-{[(4-hydroxy-2-methoxyphenyl)amino]methyl}propyl)(tert-butoxy)carboxamide (Reference Example Compound 98)

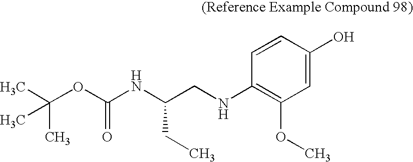

### Reference Example Compound 98

N-[(1S)-1-({[2-methoxy-4-(phenylmethoxy)phenyl]amino}methyl)propyl](tert-butoxy)carboxamide was synthesized according to the method described in the literature (Bioorg. Med. Chem., 2006, 14, 6789-6806), similarly to Reference Examples 4 to 10. N-[(1S)-1-({[2-methoxy-4-(phenylmethoxy)phenyl]amino}methyl)propyl](tert-butoxy)carboxamide (300 mg) was dissolved in tetrahydrofuran (7.5 mL) and methanol (7.5 mL). To this solution, palladium-activated carbon (10% Pd) (30 mg) was added and the mixture was stirred under hydrogen atmosphere at room temperature for 3 hours. The reaction solution was filtered through celite, and celite was washed with ethyl acetate and methanol. The filtrate was concentrated in vacuo to obtain the crude product of the title compound (Reference Example Compound 98: 233 mg). The crude product was used in the subsequent reaction without further purification.

ESI/MS m/e: 311.2 (M⁺+H, C₁₆H₂₆N₂O₄).

### Reference Example 99

### Synthesis of Phenylmethyl 2-[4-({(2S)-2-[(tert-butoxy)carbonylamino]butyl}amino)-3-methoxyphenoxy]acetate (Reference Example Compound 99)

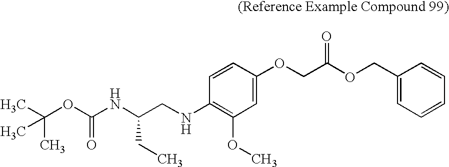

### Reference Example Compound 99

Sodium hydride (50 to 72% in mineral oil, 33 mg) was suspended in tetrahydrofuran (1.75 mL). To this suspension, a tetrahydrofuran solution (2.0 mL) of N-((1S)-1-{[(4-hydroxy-2-methoxyphenyl)amino]methyl}propyl)(tert-butoxy)carboxamide (Reference Example Compound 98: 233 mg) was added dropwise under ice-cooling and the mixture was stirred at room temperature for 5 minutes. After adding benzyl bromoacetate (131 μL) dropwise to the reaction solution, N,N-dimethylformamide (3.75 mL) was added and the mixture was stirred at room temperature for 2 hours. The reaction was quenched with a 1:1 mixed solution of saturated aqueous ammonium chloride solution and saturated saline, and extracted with ethyl acetate. The organic layer was washed with saturated saline, dried over anhydrous sodium sulfate, and filtered. The filtrate was concentrated in vacuo and the residue was purified by silica gel column chromatography to obtain the title compound (Reference Example Compound 99: 195 mg).

¹H-NMR (400 MHz, CDCl₃) δ (ppm): 7.39-7.29 (5H, m), 6.52-6.48 (2H, m), 6.35 (1H, dd, J=8.5, 2.7 Hz), 5.23 (2H, s), 4.59 (2H, s), 4.49 (1H, brs), 4.17 (1H, brs), 3.80-3.69 (4H, m), 3.18 (1H, dd, J=12.6, 4.8 Hz), 3.10-3.00 (1H, m), 1.68-1.55 (1H, m), 1.53-1.42 (10H, m), 0.97 (3H, t, J=7.4 Hz).

ESI/MS m/e: 459.2 (M⁺+H, C₂₅H₃₄N₂O₆).

### Reference Example 100

### Synthesis of phenylmethyl 2-{4-[((2S)-2-aminobutyl)amino]-3-methoxyphenoxy}acetate (Reference Example Compound 100)

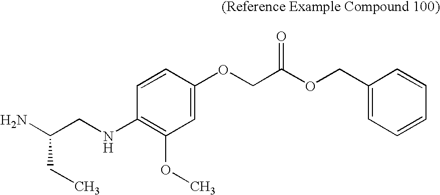

### Reference Example Compound 100

Phenylmethyl 2-[4-({(2S)-2-[(tert-butoxy)carbonylamino]butyl}amino)-3-methoxyphenoxy]acetate (Reference Example Compound 99: 195 mg) was dissolved in dichloromethane (4.3 mL). To this solution, hydrogen chloride (4 mol/L, 1,4-dioxane solution, 1.1 mL) was added and the mixture was stirred at room temperature for 2 hours. The reaction solution was concentrated in vacuo to obtain the crude product of the title compound (Reference Example Compound 100: 183 mg, hydrochloride). The crude product was used for the subsequent reaction without further purification.

ESI/MS m/e: 359.1 (M⁺+H, C₂₀H₂₆N₂O₄).

Hereinafter, the compounds described in Reference Example 101 to Reference Example 109 were synthesized using the corresponding starting materials and reagents, similarly to Reference Example 100. Their structures and M⁺+H observed by LC/MS, i.e., the measured value observed as the value of the compound molecular weight (M) with proton (H⁺) are summarized in Table 7 below.

TABLE 7

| Reference | | |
|---|---|---|
| Example No. | Structure | M⁺ + H |
| 101 | 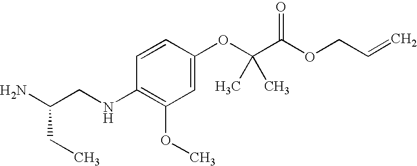 | 337.1 |
| 102 | 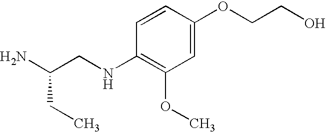 | 255.1 |
| 103 | 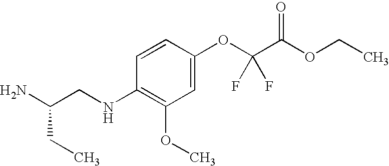 | 333.1 |
| 104 | 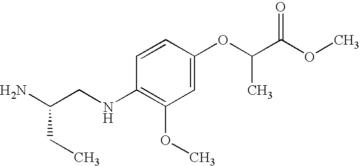 | 297.1 |
| 105 | 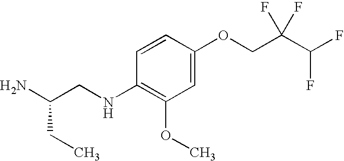 | 325.1 |
| 106 | 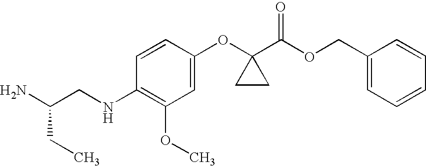 | 385.1 |
| 107 | 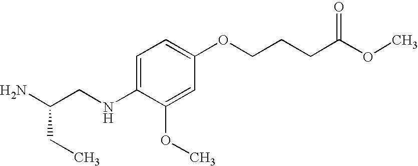 | 311.1 |
| 108 | 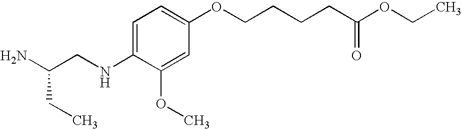 | 339.2 |
| 109 | 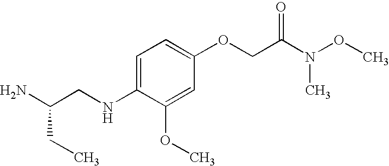 | 312.1 |

Hereinafter, the compounds described in Example 12 to Example 116 were synthesized according to the method described in Example 1, using the corresponding starting materials and reagents. Their structures, NMR spectra, and M⁺+H observed by LC/MS, i.e., the measured value observed as the value of the compound molecular weight (M) with proton (H⁺) are summarized in Table 8 below.

TABLE 8

| Example | Compound | | | |
|---|---|---|---|---|
| No. | No. | Structure | M⁺ + H | NMR |
| 12 | 12 | 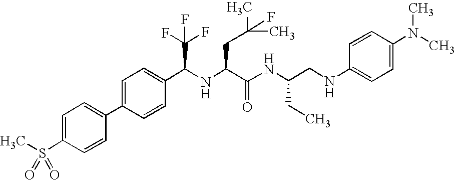 | 651.2 | |
| 13 | 13 | 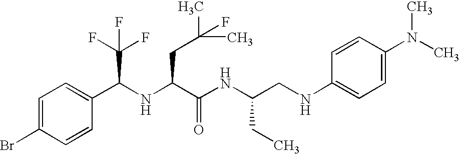 | 575.1, 577.1 | ¹H-NMR (CD₃OD) δ: 7.56 (2H, d, J = 8.8 Hz), 7.41 (4H, dd, J = 14.8, 8.9 Hz), 6.89 (2H, d, J = 9.3 Hz), 4.59 (1H, q, J = 7.4 Hz), 3.75 (2H, ddd, J = 19.6, 10.9, 4.9 Hz), 3.31- 3.29 (2H, m), 3.23 (6H, d, J = 8.5 Hz), 3.12 (2H, d, J = 6.3 Hz), 2.18- 1.97 (2H, m), 1.67-1.56 (1H, m), 1.40 (7H, dt, J = 29.3, 9.2 Hz), 0.88 (3H, t, J = 7.4 Hz). |
| 14 | 14 | 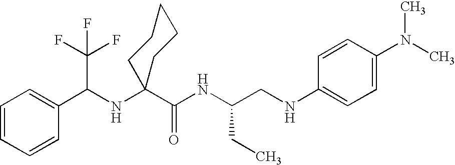 | 491.2 | |
| 15 | 15 | 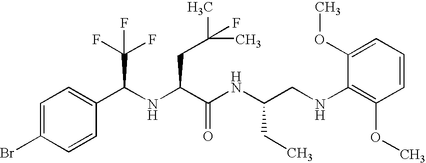 | 592.1, 594.1 | ¹H-NMR (DMSO-d₆) δ: 7.80 (1H, d, J = 7.8 Hz), 7.53 (2H, t, J = 4.3 Hz), 7.32 (2H, d, J = 8.3 Hz), 7.01 (1H, t, J = 7.8 Hz), 6.69 (2H, d, J = 8.5 Hz), 4.23 (1H, q, J = 7.8 Hz), 3.78 (6H, s), 3.59 (1H, td, J = 12.6, 7.0 Hz), 3.38 (1H, t, J = 6.2 Hz), 3.22 (1H, dd, J = 12.8, 5.0 Hz), 2.96 (1H, dd, J = 12.7, 7.3 Hz), 1.89-1.74 (2H, m), 1.40 (7H, dd, J = 21.8, 8.4 Hz), 128- 1.17 (1H, m), 0.73 (3H, t, J = 7.4 Hz). |
| 16 | 16 | 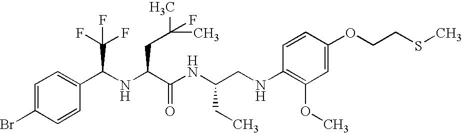 | 652.1, 654.1 | ¹H-NMR (CDCl₃) δ: 7.28 (2H, t, J = 4.1 Hz), 7.00 (2H, d, J = 8.0 Hz), 6.74 (1H, d, J = 9.3 Hz), 6.50-6.41 (3H, m), 4.15-4.00 (4H, m), 3.84 (3H, t, J = 5.6 Hz), 3.79 (1H, d, J = 8.3 Hz), 3.05 (2H, dd, J = 12.1, 4.3 Hz), 2.87 (2H, td, J = 6.8, 0.9 Hz), 2.70 (1H, dd, J = 12.0, 8.3 Hz), 2.24-2.18 (3H, m), 2.15-1.90 (2H, m), 1.58 (1H, dt, J = 20.8, 6.7 Hz), 1.52-1.42 (6H, m), 1.35 (1H, td, J = 14.5, 7.2 Hz), 1.26 (1H, td, J = 7.2, 1.1 Hz), 0.88 (3H, dd, J = 7.7, 7.0 Hz). |
| 17 | 17 | 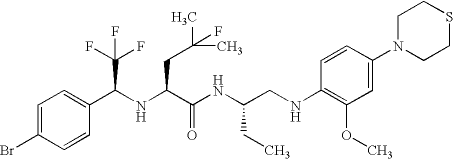 | 663.1, 665.1 | ¹H-NMR (CDCl₃) δ: 7.25 (3H, dt, J = 8.9, 2.1 Hz), 6.98 (2H, d, J = 8.3 Hz), 6.74 (1H, d, J = 9.3 Hz), 6.53 (2H, dd, J = 7.1, 2.2 Hz), 6.44 (1H, dd, J = 6.8, 2.2 Hz), 4.07-4.00 (3H, m), 3.83 (3H, s), 3.79 (1H, t, J = 7.9 Hz), 3.32 (4H, dd, J = 6.2, 4.0 Hz), 3.04 (2H, dt, J = 22.4, 8.3 Hz), 2.80 (4H, d, J = 4.9 Hz), 2.72 (1H, dd, J = 8.2 Hz), 2.15-2.06 (1H, m), 2.02-1.89 (1H, m), 1.58 (2H, tt, J = 13.3, 4.4 Hz), 1.47 (6H, t, J = 21.3 Hz), 1.38-1.30 (1H, m), 0.87 (3H, dd, J = 9.3, 5.4 Hz). |
| 18 | 18 | 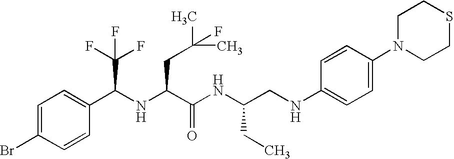 | 633.1, 635.1 | ¹H-NMR (CDCl₃) δ: 7.32 (2H, dt, J = 8.9, 2.2 Hz), 7.04 (2H, d, J = 8.3 Hz), 6.87 (2H, d, J = 8.5 Hz), 6.75 (1H, d, J = 9.3 Hz), 6.51 (2H, d, J = 8.8 Hz), 4.03-3.94 (2H, m), 3.75 (1H, d, J = 9.8 Hz), 3.32 (4H, t, J = 4.9 Hz), 3.06-3.00 (2H, m), 2.79 (4H, t, J = 5.0 Hz), 2.68 (1H, dd, J = 12.0, 8.5 Hz), 2.09 (1H, dt, J = 19.0, 8.0 Hz), 2.01-1.88 (1H, m), 1.60-1.53 (2H, m), 1.49 (3H, t, J = 9.0 Hz), 1.44 (3H, d, J = 18.5 Hz), 1.32 (1H, dd, J = 14.8, 7.0 Hz), 0.86 (3H, t, J = 7.4 Hz). |
| 19 | 19 | 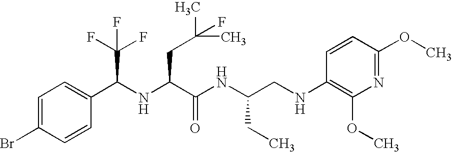 | 593.1, 595.1 | ¹H-NMR (CDCl₃) δ: 7.33 (2H, dd, J = 6.6, 1.7 Hz), 7.07 (2H, d, J = 8.3 Hz), 6.75 (2H, dd, J = 6.0, 8.7 Hz), 6.25 (1H, d, J = 8.0 Hz), 4.08 (2H, tt, J = 22.2, 6.2 Hz), 3.96 (3H, d, J = 12.0 Hz), 3.85 (3H, d, J = 9.8 Hz), 3.77 (1H, d, J = 9.8 Hz), 3.01 (2H, dt, J = 22.6, 8.4 Hz), 2.68 (1H, dd, J = 12.2, 7.8 Hz), 2.15-1.90 (2H, m), 1.60-1.38 (8H, m), 1.30 (1H, dq, J = 26.0, 6.7 Hz), 0.87 (3H, t, J = 7.4 Hz). |
| 20 | 20 | 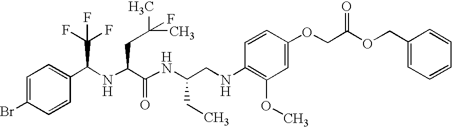 | 726.1, 728.1 | ¹H-NMR (CDCl₃) δ: 7.35 (5H, dq, J = 9.1, 2.6 Hz), 7.28 (1H, t, J = 2.2 Hz), 6.99 (2H, d, J = 8.3 Hz), 6.72 (1H, d, J = 9.3 Hz), 6.56 (1H, d, J = 2.0 Hz), 6.42-6.37 (2H, m), 5.24 (2H, s), 4.62 Hz), (2H, s), 4.03 (2H, dq, J = 17.1, 4.3 Hz), 3.79 (4H, dd, J = 8.2, 3.8 Hz), 3.03 (2H, dd, J = 12.2, 4.6 Hz), 2.67 (1H, dd, J = 12.1, 8.2 Hz), 2.08 (1H, tt, J = 23.9, 8.0 Hz), 1.94 (1H, ddd, J = 18.9, 8.7, 6.3 Hz), 1.58 (1H, ddd, J = 18.8, 9.0, 5.4 Hz), 1.52-141 (7H, m), 1.34 (1H, td, J = 14.6, 7.6 Hz), 1.27 (1H, d, J = 6.1 Hz), 0.87 (3H, t, J = 7.4 Hz). |
| 21 | 21 | 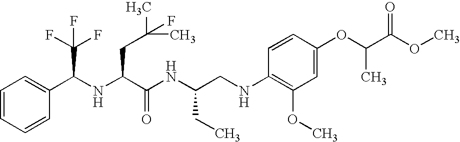 | 586.2 | ¹H-NMR (CDCl₃) δ: 7.34 (6H, s), 7.25 (1H, t, J = 6.1 Hz), 6.58 (1H, t, J = 2.3 Hz), 6.40-6.35 (1H, m), 4.76 (1H, q, J = 6.7 Hz), 4.23 (1H, td, J = 7.3, 4.8 Hz), 3.95 (1H, s), 3.82 (3H, d, J = 2.4 Hz), 3.76 (4H, t, J = 4.4 Hz), 3.28 (1H, d, J = 12.7 Hz), 2.95- 2.89 (1H, m), 2.12 (1H, tt, J = 23.5, 6.8 Hz), 1.99-1.91 (1H, m), 1.62 (3H, d, J =6.8 Hz), 1.47 (7H, dd, J = 22.1, 17.7 Hz), 1.28 (1H, tt, J = 20.5, 7.4 Hz), 0.77 (3H, t, J = 7.4 Hz). |
| 22 | 22 | 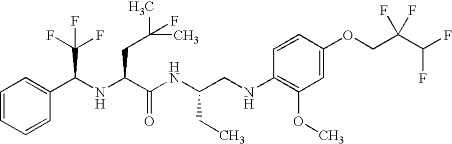 | 614.2 | ¹H-NMR (CDCl₃) δ: 7.34 (6H, dd, J = 20.5, 12.9 Hz), 7.21 (1H, d, J = 8.8 Hz), 7.02 (2H, s), 6.56 (1H, d, J = 1.5 Hz), 6.49 (1H, dd, J = 8.4, 2.1 Hz), 6.04 (1H, tt, J = 53.2, 4.6 Hz), 4.34 (2H, t, J = 12.0 Hz), 4.22 (1H, q, J = 7.3 Hz), 3.94 (1H, td, J = 15.1, 8.2 Hz), 3.85 (3H, s), 3.77 (1H, d, J = 9.0 Hz), 3.25 (1H, d, J = 12.4 Hz), 2.89 (1H, dd, J = 12.4, 9.0 Hz), 2.12 (1H, dd, J = 32.8, 15.2 Hz), 1.94 (2H, tt, J = 18.0, 6.0 Hz), 1.47 (7H, dd, J = 21.6, 18.4 Hz), 1.28 (1H, ddd, J = 30.1, 15.8, 8.2 Hz), 0.79 (3H, t, J = 7.4 Hz). |
| 23 | 23 | 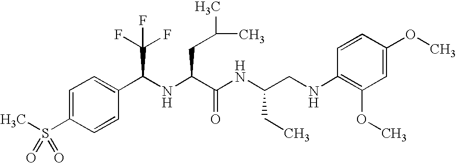 | 574.24 | |
| 24 | 24 | 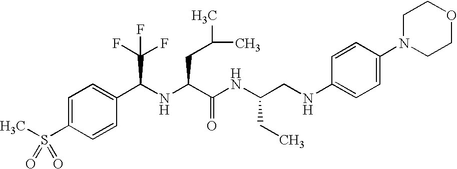 | 599.33 | |
| 25 | 25 | 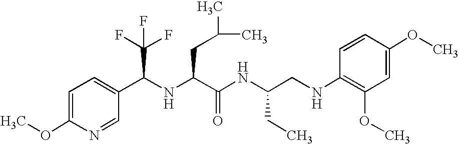 | 527.44 | |
| 26 | 26 | 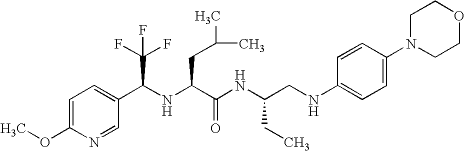 | 551.25 | |
| 27 | 27 | 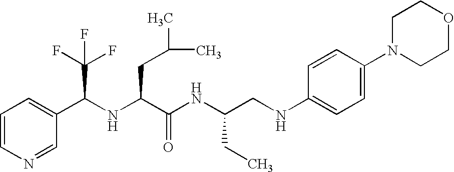 | 521.2 | |
| 28 | 29 | 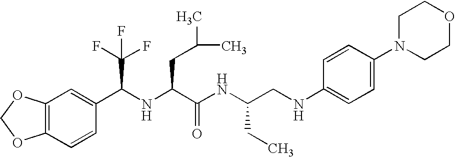 | 564.2 | |
| 29 | 30 | 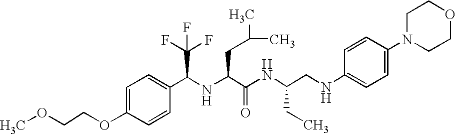 | 594.25 | |
| 30 | 31 | 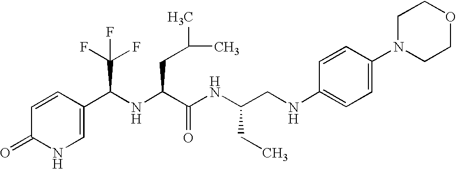 | 538.25 | |
| 31 | 32 | 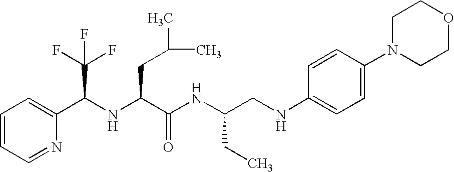 | 521.2 | |
| 32 | 33 | 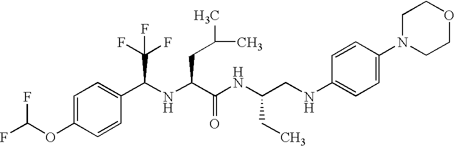 | 586.2 | |
| 33 | 34 | 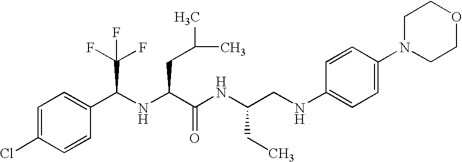 | 554.2 | |
| 34 | 35 | 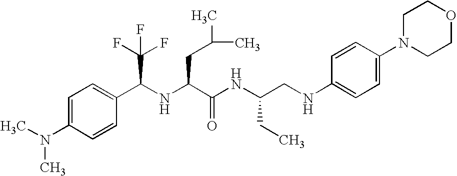 | 563.25 | |
| 35 | 36 | 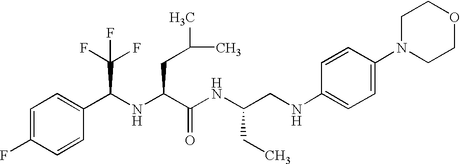 | 538.2 | |
| 36 | 37 | 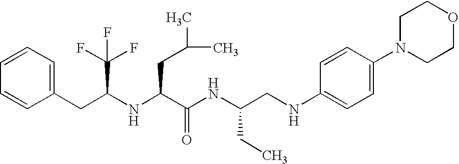 | 535.25 | |
| 37 | 38 | 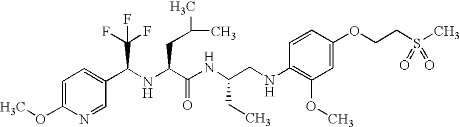 | 619.2 | |
| 38 | 39 | 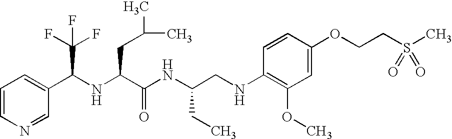 | 589.25 | |
| 39 | 40 | 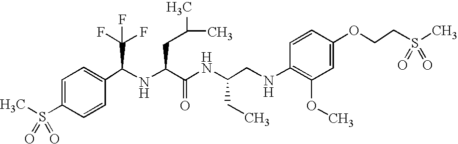 | 666.2 | |
| 40 | 41 | 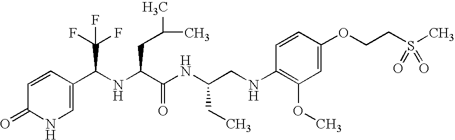 | 605.15 | |
| 41 | 42 | 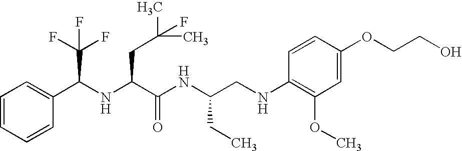 | 544.2 | ¹H-NMR (CDCl₃) δ: 7.29 (1H, dq, J = 9.5, 2.2 Hz), 7.21 (4H, tt, J = 8.2, 3.7 Hz), 6.78 (1H, d, J = 9.3 Hz), 6.50 (1H, d, J = 2.0 Hz), 6.45 (2H, d, J = 2.2 Hz), 4.13-4.06 (1H, m), 4.05-4.03 (2H, m), 4.01-3.97 (1H, m), 3.94-3.92 (2H, m), 3.82- 3.78 (4H, m), 3.07 (1H, s), 2.99 (1H, dd, J = 12.3, 5.0 Hz), 2.69 (1H, dd, J = 12.3, 2.15-1.90 (2H, m), 1.61- 1.54 (1H, m) 1.51 (3.0H, dd, J = 13.2, 8.5 Hz), 1.44 (3H, t, J = 11.0 Hz), 1.28 (1H, ddt, J = 19.5, 12.9, 3.6 Hz), 0.85 (3H, t, J = 7.4 Hz). |
| 42 | 43 | 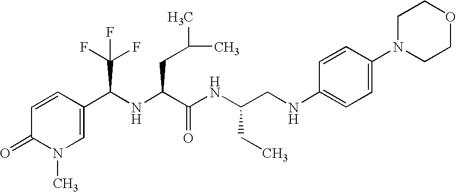 | 552.25 | ¹H-NMR (CD₃OD) δ: 7.68 (1H, d, J = 2.2 Hz), 7.62-7.44 (1H, m), 7.28 (2H, d, J = 8.8 Hz), 6.75 (2H, d, J = 8.8 Hz), 6.42 (0.9H, d, J = 9.5 Hz), 4.00-3.96 (6H, m), 3.84-3.77 (1H, m), 3.62-3.52 (5H, m), 3.46-3.40 (3H, m), 3.28-3.24 (2H, m), 3.17- 2.96 (2H, m), 2.15-1.96 (1H, m), 1.89-1.25 (4H, m), 1.21-0.79 (9H, m). |
| 43 | 44 | 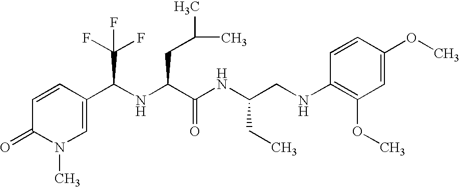 | 527.2 | ¹1-NMR (CD₃OD) δ: 7.75-7.61 (1H, m), 7.55 (1H, d, J = 9.3 Hz), 7.20 (1H, d, J = 7.6 Hz), 6.70 (1H, br s), 6.58-6.57 (1H, m), 6.51-6.46 (1H, m), 4.02 (1H, tt, J = 11.2, 5.4 Hz), 3.92 (3H, t, J = 8.4 Hz), 3.72 (4H, t, J = 27.9 Hz), 3.61-3.46 (3H, m), 3.33-3.19 (3H, m), 2.01-1.97 (1H, m), 1.82 (1H, td, J = 18.5, 14.3 Hz), 1.69-1.22 (4H, m), 1.07-0.72 (9H, m). |
| 44 | 45 | 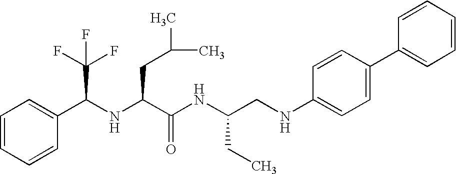 | 512.2 | |
| 45 | 46 | 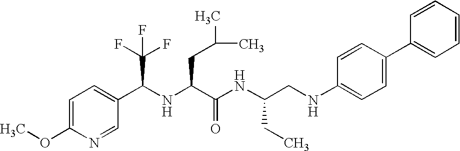 | 543.25 | |
| 46 | 47 | 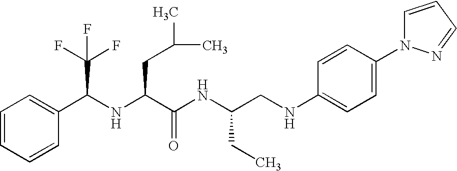 | 502.2 | ¹H-NMR (CD₃OD) δ: 7.89-7.88 (1H, m), 7.76-7.74 (1H, m), 7.55 (1H, d, J = 1.7 Hz), 7.34-7.23 (5H, m), 6.64 (2H, dt, J = 15.4, 6.3 Hz), 6.37 (1H, t, J = 2.1 Hz), 4.02 (1H, dt, J = 34.7, 12.1 Hz), 3.80 (1H, t, J = 5.5 Hz), 3.34 (1H, dd, J = 8.5, 5.9 Hz), 3.01- 2.57 (2H, m), 1.73 (1H, dt, J = 20.4, 6.8 Hz), 1.62-1.52 (1H, m), 1.41- 1.21 (3H, m), 0.92-0.73 (10H, m). |
| 47 | 48 | 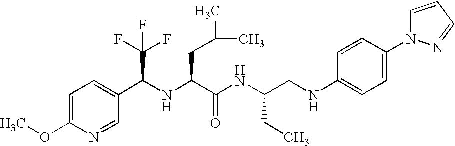 | 533.2 | |
| 48 | 49 | 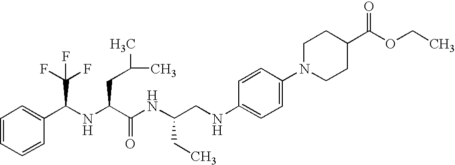 | 591.2 | |
| 49 | 50 | 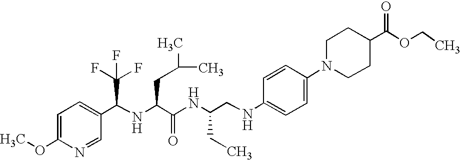 | 622.3 | |
| 50 | 51 | 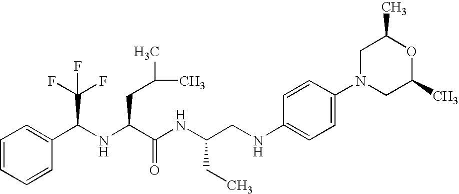 | 549.2 | ¹H-NMR (CDCl₃) δ: 7.37-7.29 (7H, m), 6.67 (2H, dd, J = 8.9, 4.3 Hz), 4.11 (3H, dt, J = 26.6, 7.9 Hz), 3.96- 3.92 (2H, m), 3.59 (3H, d, J = 11.5 Hz), 3.03 (1H, dd, J = 12.7, 3.9 Hz), 2.87 (3H, t, J = 11.3 Hz), 2.66 (1H, dd, J = 12.4, 8.8 Hz), 1.76-1.45 (3H, m), 1.31-1.27 (8H, m), 1.01-0.96 (6H, m), 0.85 (3H, t, J = 7.3 Hz). |
| 51 | 52 | 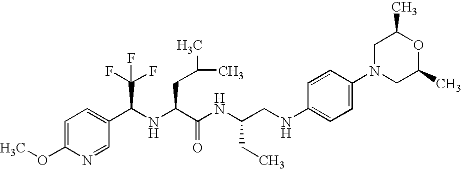 | 580.2 | |
| 52 | 53 | 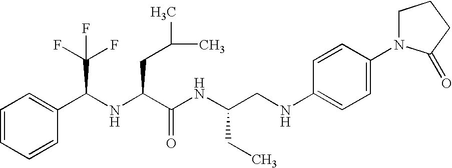 | 519.2 | |
| 53 | 54 | 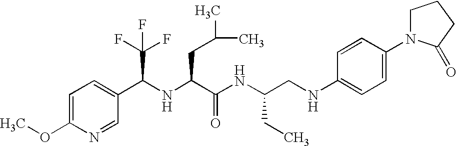 | 550.2 | |
| 54 | 55 | 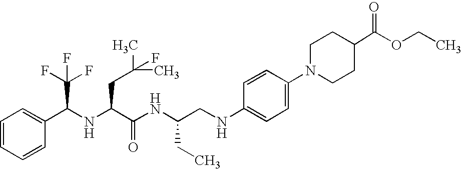 | 609.25 | |
| 55 | 56 | 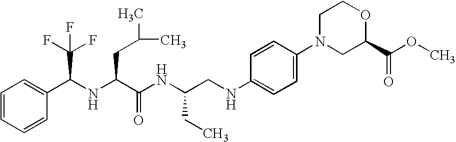 | 579.2 | |
| 56 | 57 | 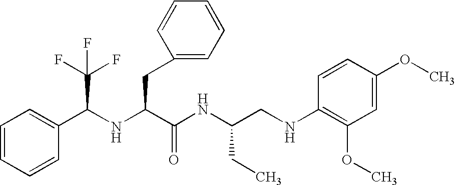 | 530.2 | ¹H-NMR (CD₃OD) δ: 7.33 (5H, s), 7.20 (4H, d, J = 4.4 Hz), 7.18-7.12 (1H, m), 7.04 (1H, dd, J = 8.8, 2.2 Hz), 6.70 (1H, d, J = 2.4 Hz), 6.59 (1H, dd, J = 8.8, 2.7 Hz), 4.10 (1H, q, J = 7.6 Hz), 3.89 (3H, s), 3.81 (3H, s), 3.62 (1H, ddt, J = 11.2, 6.2, 2.7 Hz), 3.49 (1H, t, J = 7.1 Hz), 3.05-2.88 (3H, m), 2.75 (1H, dd, J = 12.7, 7.1 Hz), 1.56-1.46 (1H, m), 1.39-1.28 (1H, m), 0.77 (3H, t, J = 7.4 Hz). |
| 57 | 58 | 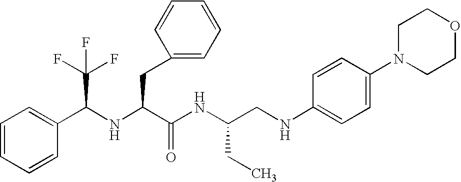 | 556.3 | ¹H-NMR (CD₃OD) δ: 7.32-7.16 (12H, m), 6.76 (2H, d, J = 7.6 Hz), 4.07 (1H, q, J = 7.5 Hz), 3.98 (4H, s), 3.70 (1H, s), 3.47-3.51 (4H, m), 2.98-2.82 (5H, m), 1.61-1.51 (1H, m), 1.33-1.24 (1H, m), 0.80 (3H, t, J = 7.4 Hz). |
| 58 | 59 | 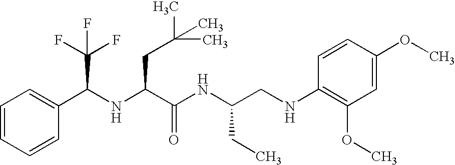 | 510.2 | ¹H-NMR (CD₃OD) δ: 7.44-7.30 (5H, m), 7.06 (1H, s), 6.68-6.57 (2H, m), 4.09 (1H, q, J = 7.6 Hz), 3.90 (3H, s), 3.81 (3H, s), 3.67 (1H, s), 3.48 (1H, t, J = 6.2 Hz), 3.20 (1H, s), 3.07-2.98 (1H, m), 1.73-1.39 (4H, m), 1.02 (9H, s), 0.90 (3H, t, J = 7.6 Hz). |
| 59 | 60 | 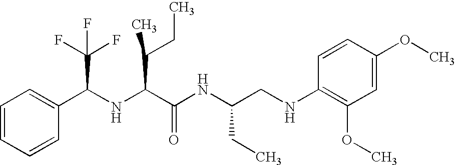 | 496.2 | |
| 60 | 61 | 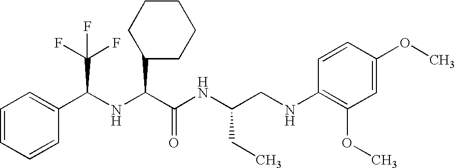 | 522.2 | |
| 61 | 28 | 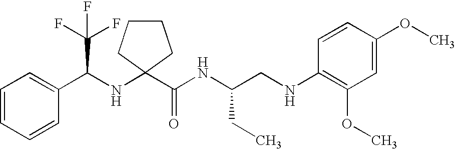 | 494.2 | |
| 62 | 62 | 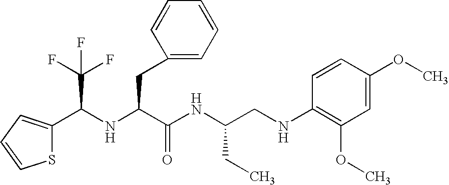 | 536.1 | |
| 63 | 63 | 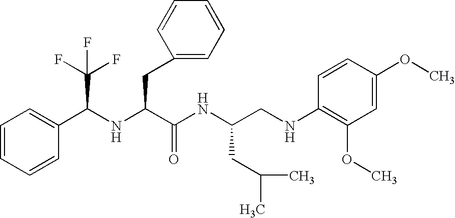 | 558.3 | |
| 64 | 64 | 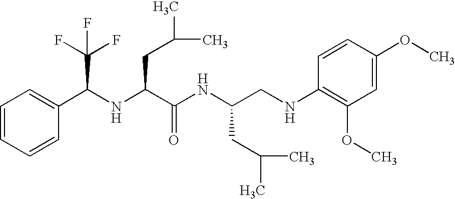 | 524.2 | |
| 65 | 65 | 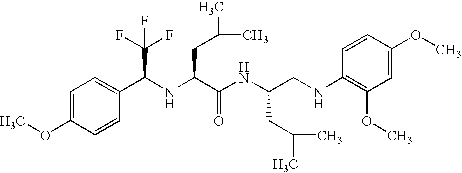 | 554.3 | |
| 66 | 66 | 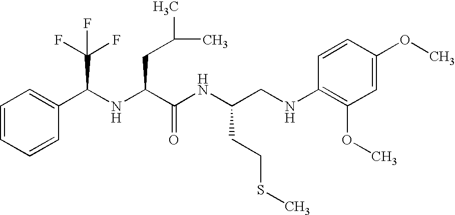 | 542.2 | |
| 67 | 67 | 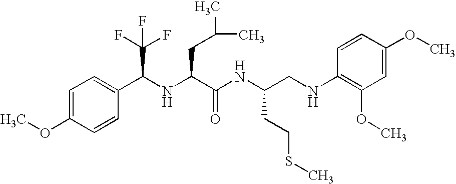 | 572.2 | |
| 68 | 68 | 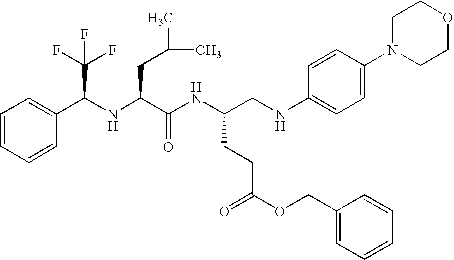 | 655.3 | |
| 69 | 69 | 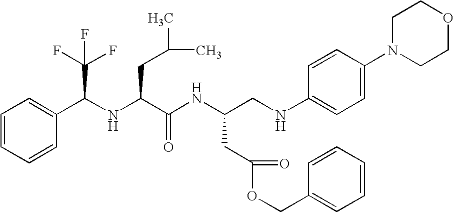 | 641.3 | |
| 70 | 70 | 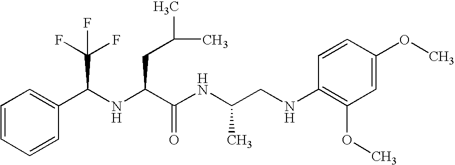 | 482.2 | |
| 71 | 71 | 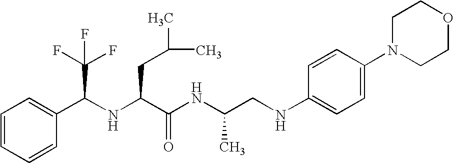 | 506.3 | |
| 72 | 72 | 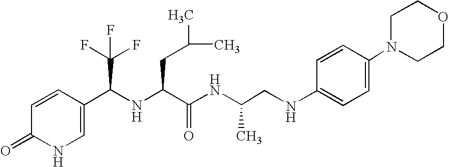 | 524.3 | |
| 73 | 73 | 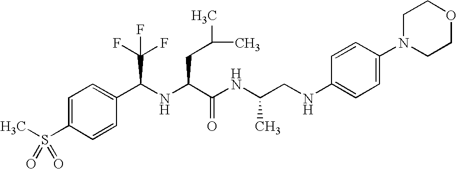 | 585.3 | |
| 74 | 74 | 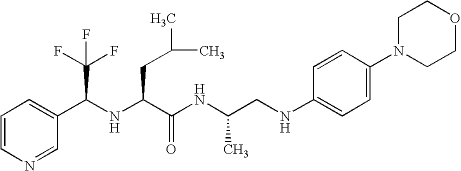 | 508.3 | |
| 75 | 75 | 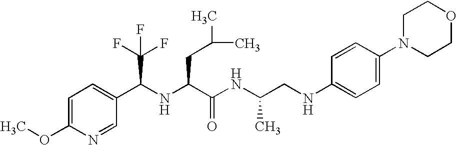 | 538.3 | |
| 76 | 76 | 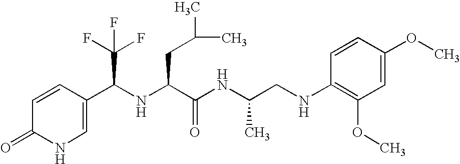 | 499.2 | |
| 77 | 77 | 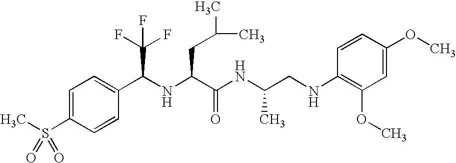 | 560.2 | |
| 78 | 78 | 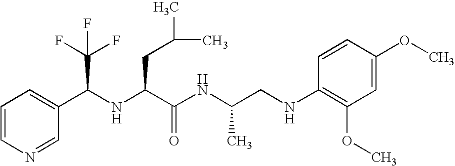 | 483.2 | |
| 79 | 79 | 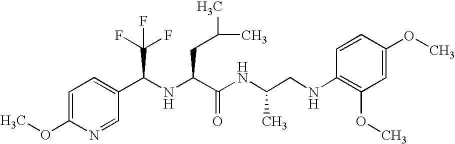 | 513.2 | |
| 80 | 80 | 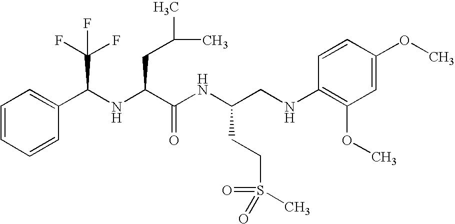 | 574.2 | |
| 81 | 81 | 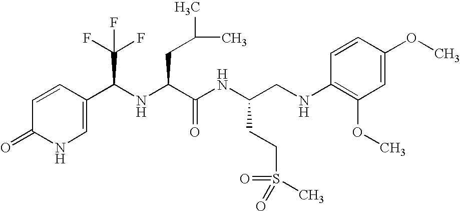 | 591.5 | |
| 82 | 82 | 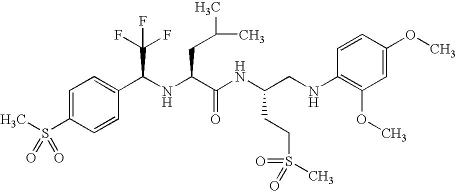 | 652.5 | |
| 83 | 83 | 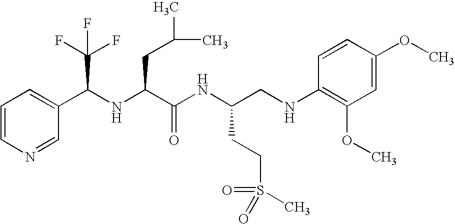 | 575.2 | |
| 84 | 84 | 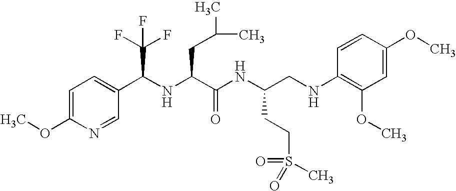 | 605.2 | |
| 85 | 85 | 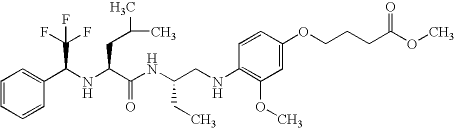 | 582.3 | |
| 86 | 86 | 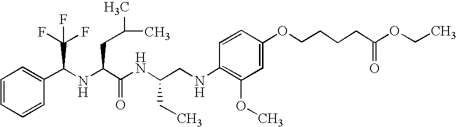 | 610.3 | |
| 87 | 87 | 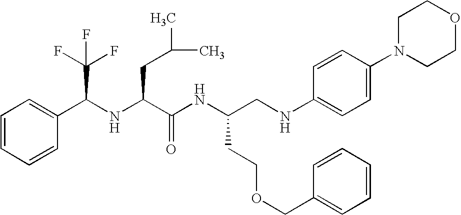 | 627.3 | |
| 88 | 88 | 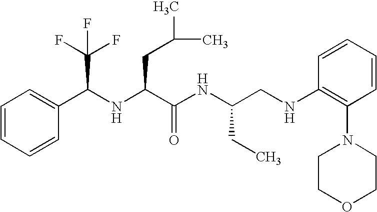 | 521.3 | |
| 89 | 89 | 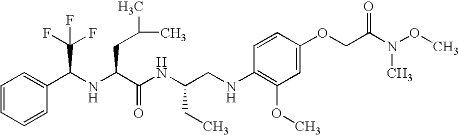 | 583.3 | |
| 90 | 90 | 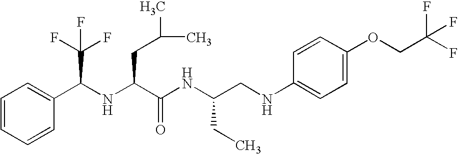 | 534.2 | |
| 91 | 91 | 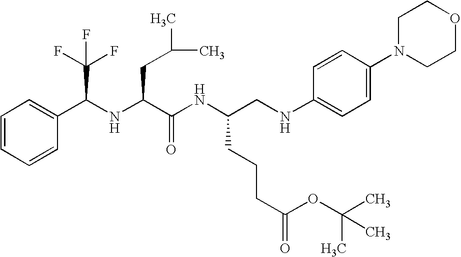 | 635.3 | |
| 92 | 92 | 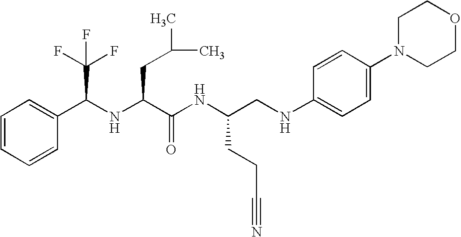 | 546.2 | |
| 93 | 93 | 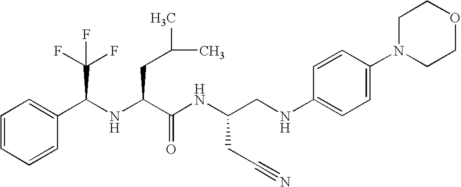 | 532.2 | |
| 94 | 94 | 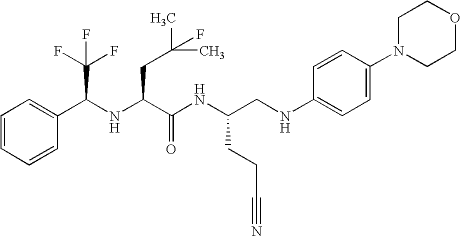 | 564.1 | |
| 95 | 95 | 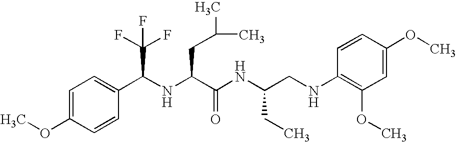 | 526.2 | |
| 96 | 96 | 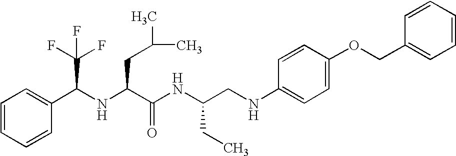 | 542.3 | |
| 97 | 97 | 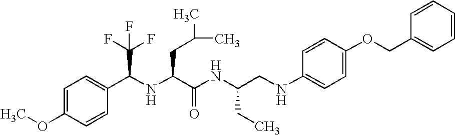 | 572.3 | |
| 98 | 98 | 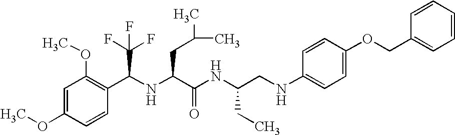 | 556.3 | |
| 99 | 99 | 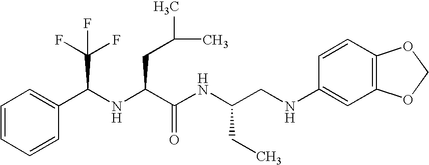 | 480.2 | |
| 100 | 100 | 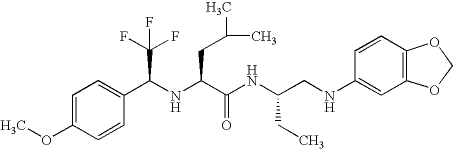 | 510.1 | |
| 101 | 101 | 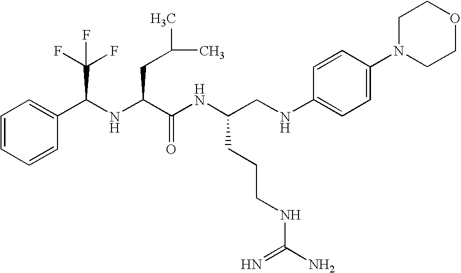 | 592.3 | |
| 102 | 102 | 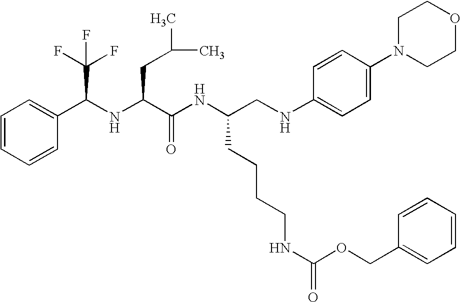 | 698.3 | |
| 103 | 103 | 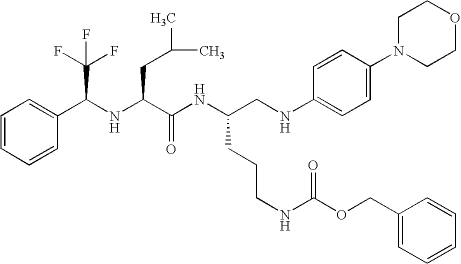 | 683.3 | |
| 104 | 104 | 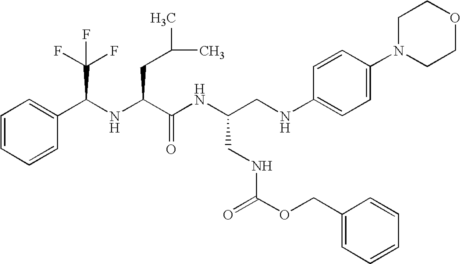 | 656.2 | |
| 105 | 105 | 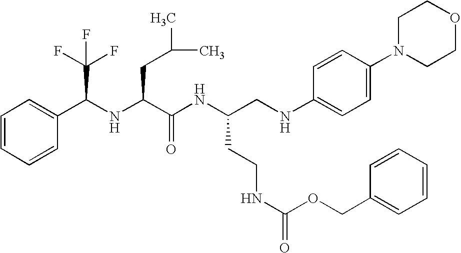 | 670.2 | |
| 106 | 106 | 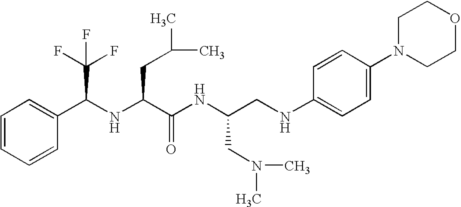 | 550.3 | |
| 107 | 107 | 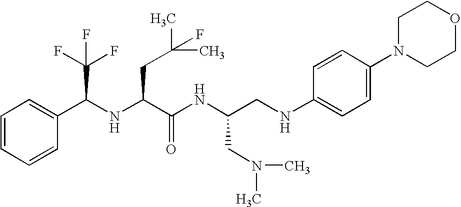 | 568.2 | |
| 108 | 108 | 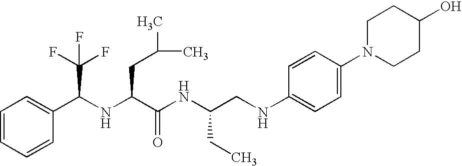 | 535.3 | |
| 109 | 109 | 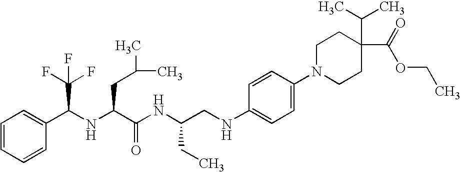 | 633.3 | |
| 110 | 110 | 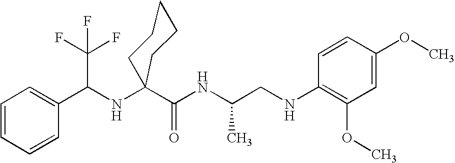 | 494.2 | ¹H-NMR (CD₃OD) δ: 7.42 (2H, t, J = 7.3 Hz), 7.36-7.21 (4H, m), 6.74 (1H, s), 6.63 (1H, s), 4.25 (1H, q, J = 8.1 Hz), 3.94 (3H, s), 3.83 (3H, s), 3.68 (0.5H, dd, J = 12.2, 6.1 Hz), 3.10 (1H, s), 2.89 (0.5H, s), 1.85 (2H, ddt, J = 33.9, 15.0, 5.3 Hz), 1.72- 1.24 (8H, m), 1.19 (1H, d, J = 2.7 Hz), 0.98 (1H, d, J = 6.8 Hz). |
| 111 | 111 | 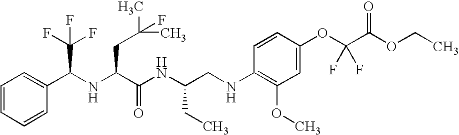 | 622.2 | ¹H-NMR (CDCl₃) δ: 7.28 (1H, dt, J = 7.0, 1.9 Hz), 7.18 (4H, ddd, J = 15.8, 9.0, 3.1 Hz), 6.88 (1H, d, J = 9.0 Hz), 6.77 (1H, dd, J = 8.5, 2.4 Hz), 6.68 (1H, d, J = 2.4 Hz), 6.47 (1H, d, J = 8.5 Hz), 4.37 (2H, q, J = 7.2 Hz), 4.03 (2H, tt, J = 17.0, 5.4 Hz), 3.82 (4H, t, J = 6.2 Hz), 3.01 (1H, dd, J = 12.3, 4.8 Hz), 2.86 (6H, s), 2.68 (1H, dd, J = 12.2, 8.3 Hz), 2.15-1.91 (2H, m), 1.58 (1H, dt, J = 12.1, 4.7 Hz), 1.48 (6H, dd, J = 23.2, 22.2 Hz), 1.37 (3H, t, J = 7.2 Hz), 1.28 (1H, ddt, J = 19.4, 11.0, 3.9 Hz), 0.85 (3H, t, J = 7.4 Hz). |
| 112 | 112 | 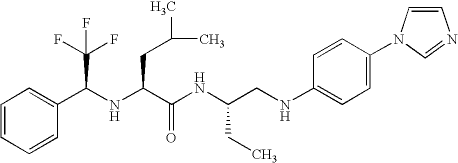 | 502.15 | |
| 113 | 113 | 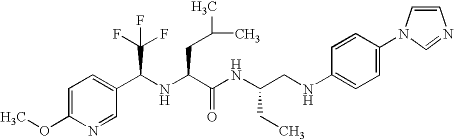 | 533.25 | |
| 114 | 114 | 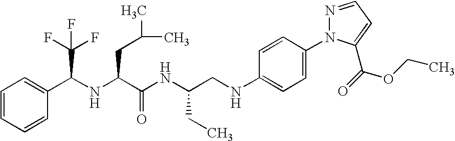 | 574.2 | |
| 115 | 115 | 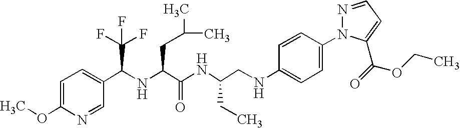 | 605.2 | |
| 116 | 116 | 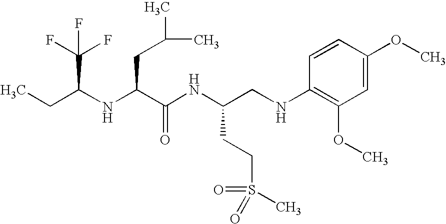 | 526.2 | |

### Example 117

### Synthesis of 2-{4-[((2S)-2-{(2S)-2-[((1S)-2,2,2-trifluoro-1-phenylethyl)amino]-4-fluoro-4-methylpentanoylamino}butyl)amino]-3-methoxyphenoxy}acetic acid (117)

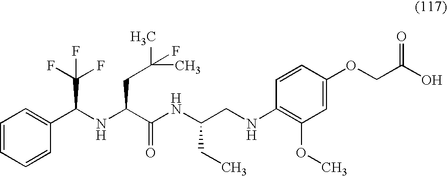

Phenylmethyl 2-{4-[((2S)-2-{(2S)-2-{[(1S)-2,2,2-trifluoro-1-(4-bromophenyl)ethyl]amino}-4-fluoro-4-methylpentanoylamino}butyl)amino]-3-methoxyphenoxy}acetate (20: 28.5 mg) was dissolved in tetrahydrofuran (784 μL). To this solution, palladium-activated carbon (10% Pd) (3 mg) was added and the mixture was stirred under hydrogen atmosphere at room temperature for 1 hour. The reaction solution was filtered through celite, and celite was washed with ethyl acetate and methanol. The filtrate was concentrated in vacuo and the residue was purified by high performance liquid chromatography to obtain the title compound (117: 15.1 mg, trifluoroacetate).

¹H-NMR (400 MHz, CD₃OD) δ (ppm): 7.33 (2H, dd, J=6.6, 4.9 Hz), 7.29 (3H, dq, J=7.0, 2.1 Hz), 7.10 (1H, d, J=8.0 Hz), 6.74 (1H, s), 6.55 (1H, d, J=7.3 Hz), 4.67 (2H, s), 4.16 (1H, q, J=7.6 Hz), 3.87 (3H, s), 3.65-3.59 (1H, m), 3.54 (1H, dd, J=7.6, 5.1 Hz), 3.27-3.25 (1H, m), 3.19 (1H, d, J=11.0 Hz), 2.99 (1H, t, J=9.4 Hz), 2.04-1.83 (2H, m), 1.55-1.48 (1H, m), 1.40 (7H, dd, J=21.7, 9.8 Hz), 0.84 (3H, t, J=7.4 Hz).

ESI/MS m/e: 558.2 (M⁺+H, C₂₇H₃₅F₄N₃O₅).

Hereinafter, the compounds described in Example 118 to Example 130 were synthesized according to the method described in Example 117, using the corresponding starting materials and reagents. Their structures, NMR spectra, and M⁺+H observed by LC/MS, i.e., the measured value observed as the value of the compound molecular weight (M) with proton (H⁺) are summarized in Table 9 below.

TABLE 9

| Example | Compound | | | |
|---|---|---|---|---|
| No. | No. | Structure | M⁺ + H | NMR |
| 118 | 118 | 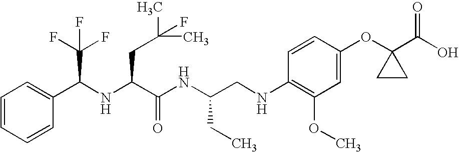 | 584.2 | ¹H-NMR (CDCl₃) δ: 7.33 (6H, ddt, J = 18.6, 10.4, 4.0 Hz), 7.01 (1H, d, J = 8.5 Hz), 6.49 (1H, d, J = 2.4 Hz), 6.45 (1H, dd, J = 8.8, 2.4 Hz), 4.30 (1H, q, J = 7.3 Hz), 4.00-3.96 (1H, m), 3.77 (1H, dd, J = 9.6, 2.8 Hz), 3.64 (3H, s), 3.09 (1H, dd, J = 12.7, 3.2 Hz), 2.86 (1H, dd, J = 12.4, 9.0 Hz), 2.17-2.02 (1H, m), (1H, m), 1.94 (1H, ddt, J = 20.1, 10.5, 4.0 Hz), 1.67 (2H, dd, J = 7.3, 4.1 Hz), 1.47 (7H, dt, J = 25.3, 7.6 Hz), 1.26 (3H, ddt, J = 25.8, 14.7, 4.7 Hz), 0.76 (3H, t, J = 7.4 Hz). |
| 119 | 119 | 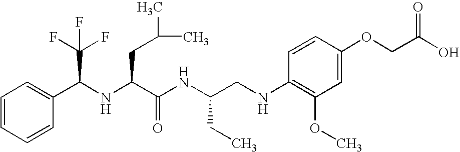 | 540.2 | ¹H NMR (CD₃OD) δ: 7.53-7.21 (6H, m), 6.80 (1H, d, J = 2.4 Hz), 6.60 (1H, dd, J = 8.8, 4.4 Hz), 4.72 (2H, s), 4.21-4.02 (1H, m), 3.97-3.94 (3H, m), 3.74-3.65 (1H, m), 3.43-3.23 (3H, m), 3.08-3.01 (1H, m), 1.93- 1.66 (1H, m), 1.62-1.28 (4H, m), 1.00-0.83 (9H, m). |
| 120 | 120 | 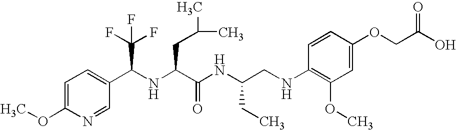 | 571.2 | |
| 121 | 121 | 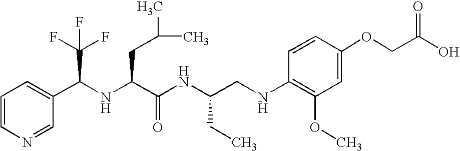 | 541.2 | |
| 122 | 122 | 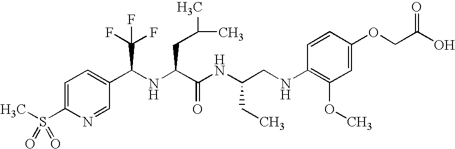 | 618.1 | |
| 123 | 123 | 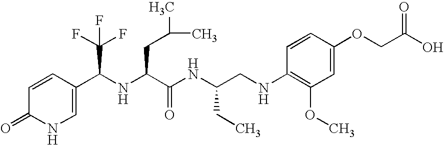 | 557.12 | |
| 124 | 124 | 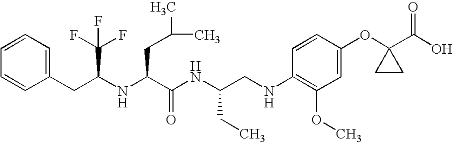 | 580.1 | |
| 125 | 125 | 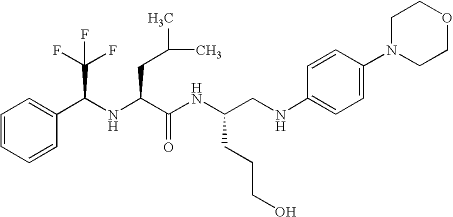 | 550.3 | |
| 126 | 126 | 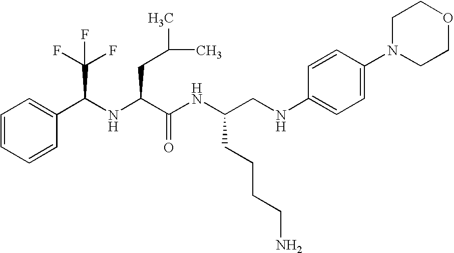 | 564.3 | |
| 127 | 127 | 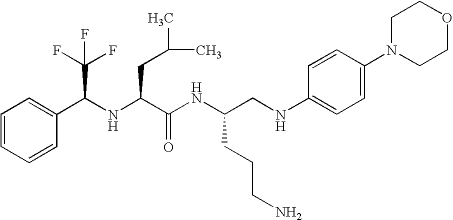 | 550.3 | |
| 128 | 128 | 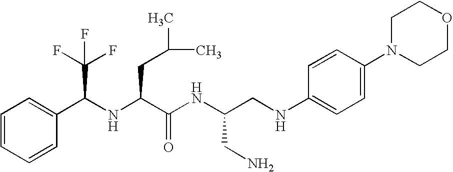 | 522.2 | |
| 129 | 129 | 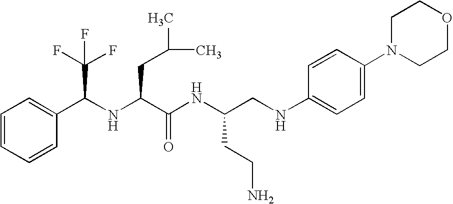 | 536.2 | |
| 130 | 130 | 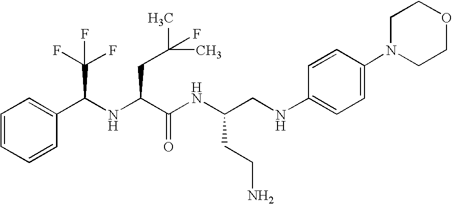 | 554.2 | |

### Example 131

### Synthesis of 2-{4-[((2S)-2-{(2S)-2-[((1S)-2,2,2-trifluoro-1-phenylethyl)amino]-4-fluoro-4-methylpentanoylamino}butyl)amino]-3-methoxyphenoxy}-2-methylpropanoic acid (131)

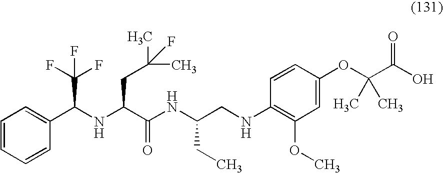

2-Propenyl 2-{4-[((2S)-2-{(2S)-2-[((1S)-2,2,2-trifluoro-1-phenylethyl)amino]-4-fluoro-4-methylpentanoylamino}butyl)amino]-3-methoxyphenoxy}-2-methylpropanoate was synthesized according to the method described in Example 1. 2-Propenyl 2-{4-[((2S)-2-{(2S)-2-[((1S)-2,2,2-trifluoro-1-phenylethyl)amino]-4-fluoro-4-methylpentanoylamino}butyl)amino]-3-methoxyphenoxy}-2-methylpropanoate (23 mg) was dissolved in acetonitrile (500 μL) and ethyl acetate (500 μL). To this solution, pyrrolidine (4.6 μL), tetrakis(triphenylphosphine)palladium (4.2 mg) and triphenylphosphine (1.9 mg) was added. After adding water (50 μL), the mixture was stirred at room temperature for 30 minutes. The reaction was quenched with a 1:1 mixed solution of saturated aqueous ammonium chloride solution and saturated saline, and extracted with ethyl acetate. The organic layer was washed with saturated saline, dried over anhydrous sodium sulfate, and filtered. The filtrate was concentrated in vacuo and the residue was purified by high performance liquid chromatography to obtain the title compound (131: 16.5 mg, trifluoroacetate).

¹H-NMR (400 MHz, CDCl₃) δ (ppm): 7.41 (1H, d, J=7.1 Hz), 7.34 (5H, d, J=10.0 Hz), 7.08 (1H, d, J=8.5 Hz), 6.50 (1H, d, J=2.4 Hz), 6.36-6.26 (7H, m), 4.24 (1H, q, J=7.4 Hz), 3.94 (1H, t, J=7.3 Hz), 3.76 (1H, dd, J=9.4, 2.8 Hz), 3.71 (3H, s), 3.21 (1H, t, J=6.2 Hz), 2.94 (1H, dd, J=12.7, 9.0 Hz), 2.11 (1H, tt, J=23.3, 6.4 Hz), 2.00-1.90 (1H, m), 1.60 (6.3H, s), 1.49 (4H, d, J=17.1 Hz), 1.43 (3H, d, J=17.1 Hz), 1.28 (1H, dq, J=24.5, 6.2 Hz), 0.77 (3H, t, J=7.3 Hz).

ESI/MS m/e: 586.2 (M⁺+H, C₂₉H₃₉F₄N₃O₅).

### Example 132

### Synthesis of 2-(4-{[(2S)-2-((2S)-2-{[(1S)-2,2,2-trifluoro-1-benzylethyl]amino}-4-fluoro-4-methylpentanoylamino)butyl]amino}-3-methoxyphenoxy)-2-methylpropanoic acid (132)

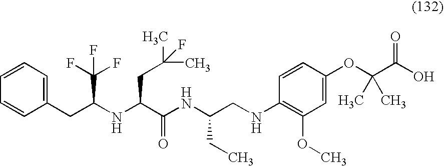

2-(4-{[(2S)-2-((2S)-2-{[(1S)-2,2,2-trifluoro-1-benzylethyl]amino}-4-fluoro-4-methylpentanoylamino)butyl]amino}-3-methoxyphenoxy)-2-methylpropanoic acid (132) was synthesized according to the method described in Example 132, using 2-propenyl 2-(4-{[(2S)-2-((2S)-2-{[(1S)-2,2,2-trifluoro-1-benzylethyl]amino}-4-fluoro-4-methylpentanoylamino)butyl]amino}-3-methoxyphenoxy)-2-methylpropanoate as a starting material.

ESI/MS m/e: 600.2 (M⁺+H, C₃₀H₄₁F₄N₃O₅).

### Example 133

### Synthesis of (2S)—N-[(1S)-1-({[4-(carbamoylmethoxy)-2-methoxyphenyl]amino}methyl)propyl]-2-[((1S)-2,2,2-trifluoro-1-phenylethyl)amino]-4-fluoro-4-methylpentanamide (133)

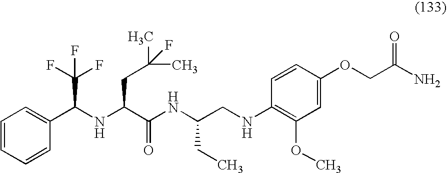

2-{4-[((2S)-2-{(2S)-2-[((1S)-2,2,2-trifluoro-1-phenylethyl)amino]-4-fluoro-4-methylpentanoylamino}butyl)amino]-3-methoxyphenoxy}acetic acid (117: 30 mg) was dissolved in N,N-dimethylformamide (538 mL). To this solution, HATU (22.5 mg), ammonia (28% aqueous solution, 4 μL) and triethylamine (7.5 μL) were added under ice-cooling and the mixture was stirred under ice-cooling for 3 hours. The reaction was quenched with acetic acid (30 μL) and the solution was purified by high performance liquid chromatography to obtain the title compound (133: 11.9 mg, trifluoroacetate).

¹H-NMR (400 MHz, CDCl₃) δ (ppm): 7.36-7.29 (6H, m), 7.20 (1H, d, J=8.8 Hz), 6.70 (1H, s), 6.55 (1H, d, J=2.7 Hz), 6.48 (1H, dd, J=8.8, 2.7 Hz), 6.41 (I H, s), 4.50 (2H, s), 4.23 (1H, q, J=7.5 Hz), 3.98-3.91 (1H, m), 3.84 (3H, s), 3.78-3.71 (2H, m), 3.25 (1H, dd, J=12.7, 2.7 Hz), 2.89 (1H, dd, J=12.6, 8.9 Hz), 2.11 (1H, tt, J=22.3, 6.5 Hz), 1.96 (1H, dt, J=22.4, 7.1 Hz), 1.55-1.38 (7H, m), 1.33-1.21 (1H, m), 0.79 (3H, t, J=7.4 Hz).

ESI/MS m/e: 557.2 (M⁺+H, C₂₇H₃₆F₄N₄O₄).

Hereinafter, the compounds described in Example 134 to Example 137 were synthesized according to the method described in Example 133, using the corresponding starting materials and reagents. Their structure, NMR spectra, and M⁺+H observed by LC/MS, i.e., the measured value observed as the value of the compound molecular weight (M) with proton (H⁺) added are summarized in Table 10 below.

TABLE 10

| Example | Compound | | | |
|---|---|---|---|---|
| No. | No. | Structure | M⁺ + H | NMR |
| 134 | 134 | 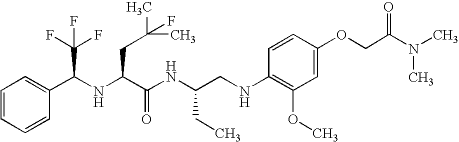 | 585.2 | ¹H-NMR (CDCl₃) δ: 7.29 (9H, ddt, J = 44.0, 23.0, 8.2 Hz), 6.63 (1H, d, J = 2.7 Hz), 6.44 (1H, dd, J = 8.8, 2.4 Hz), 4.70 (2H, s), 4.23 (1H, q, J = 7.4 Hz), 3.92 (1H, d, J = 6.3 Hz), 3.77 (4H, dd, J = 22.2, 10.0 Hz), 3.25 (1H, dd, J = 12.6, 2.1 Hz), 3.08 (3H, s), 2.95 (4H, dd, J = 20.1, 16.2 Hz), 2.18-1.90 (2H, m), 1.47 (7H, dd, J = 21.8, 17.9 Hz), 1.30- 1.23 (1H, m), 0.77 (3H, t, J = 7.4 Hz). |
| 135 | 135 | 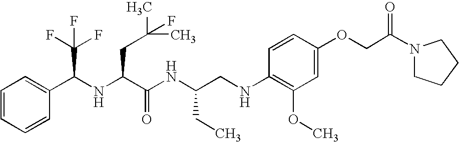 | 611.3 | ¹H-NMR (CDCl₃) δ: 7.33 (6H, dd, J = 16.5, 13.8 Hz), 7.14 (1H, d, J = 8.5 Hz), 6.62 (3H, s), 6.43 (1H, d, J = 8.8 Hz), 4.62 (2H, s), 4.21 (1H, q, J = 7.1 Hz), 3.98-3.91 (1H, m), 3.81 (3H, s), 3.75 (1H, d, J = 8.8 Hz), 3.51 (4H, dt, J = 12.4, 5.5 Hz), 3.20 (1H, d, J = 12.4 Hz), 2.94-2.87 (1H, m), 2.16-1.86 (6H, m), 1.47 (7H, t, J = 20.5 Hz), 1.30-1.23 (1H, m), 0.78 (3H, t, J = 7.1 Hz). |
| 136 | 136 | 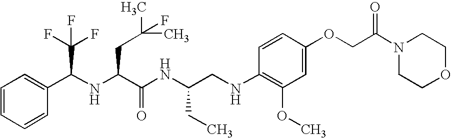 | 627.2 | ¹H-NMR (CDCl₃) δ: 7.39-7.30 (6H, m), 7.18 (1H, d, J = 8.8 Hz), 6.78 (3H, s), 6.62 (1H, t, J = 2.6 Hz), 6.47 (1H, td, J = 5.7, 2.9 Hz), 4.69 (2H, s), 4.22 (1H, q, J = 7.3 Hz), 3.95 (1H, t, J = 6.8 Hz), 3.82 (3H, s), 3.76 (1H, d, J = 9.3 Hz), 3.63 (8H, dd, J = 31.8, 17.0 Hz), 3.24 (1H, d, J = 12.7 Hz), 2.90 (1H, dd, J = 12.3, 9.1 Hz), 2.17-1.90 (2H, m), 1.47 (7H, dd, J = 21.5, 18.8 Hz), 1.32-1.22 (1H, m), 0.78 (3H, t, J = 7.3 Hz). |
| 137 | 137 | 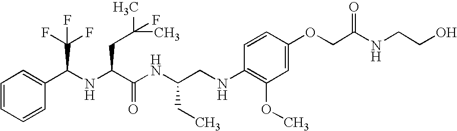 | 601.2 | ¹H-NMR (CDCl₃) δ: 7.30 (1H, tt, J = 7.0, 2.0 Hz), 7.23 (4H, dt, J = 18.8, 5.5 Hz), 7.01 (1H, s), 6.75 (1H, d, J = 9.5 Hz), 6.45 (3H, ddd, J = 21.4, 10.1, 4.0 Hz), 4.47 (2H, s), 4.12-3.96 (3H, m), 3.83 (3H, s), 3.79 (1H, d, J = 10.0 Hz), 3.74 (2H, t, J = 5.0 Hz), 5.0 Hz), 3.51 (2H, dd, J = 9.9, 5.7 Hz), 3.08 (1H, t, J = 9.1 Hz), 3.00 (1H, dd, J = 12.4, 4.9 Hz), 2.66-2.57 (2H, m), 2.14-1.90 (2H, m), 1.50 (7H, ddd, J = 36.0, 15.5, 12.7 Hz), 1.32-1.20 (1H, m), 0.84 (3H, t, J = 7.4 Hz). |

### Example 138

### Synthesis of (2S)-2-{[(1S)-1-(4-bromophenyl)-2,2,2-trifluoroethyl]amino}-N-{(1S)-1-[({2-methoxy-4-[2-(methylsulfinyl)ethoxy]phenyl}amino)methyl]propyl}-4-fluoro-4-methylpentanamide (138-1) and (2S)-2-{[(1S)-1-(4-bromophenyl)-2,2,2-trifluoroethyl]amino}-N-{(1S)-1-[({2-methoxy-4-[2-(methylsulfonyl)ethoxy]phenyl}amino)methyl]propyl}-4-fluoro-4-methylpentanamide (138-2)

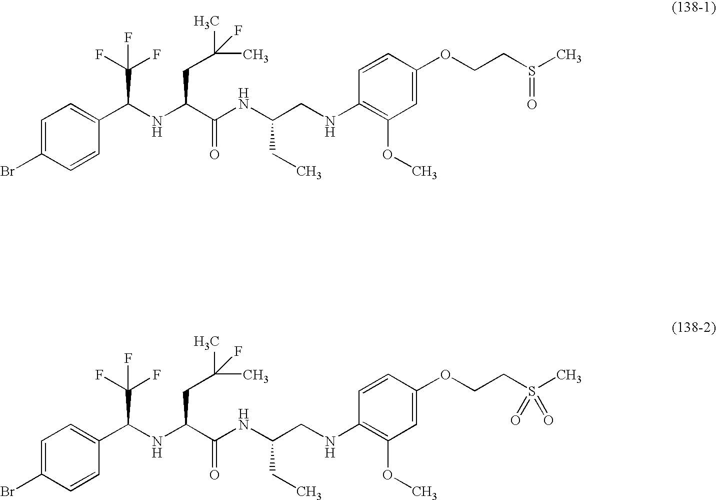

(2S)-2-{[(1S)-1-(4-bromophenyl)-2,2,2-trifluoroethyl]amino}-N-[(1S)-1-({[2-methoxy-4-(2-methylthioethoxy)phenyl]amino}methyl)propyl]-4-fluoro-4-methylpentanamide (16: 19.7 mg) was dissolved in acetone (450 μL) and water (150 μL). To this solution, N-methylmorpholine-N-oxide (10.6 mg) and osmium tetraoxide (2.5 wt %, tert-butanol solution, 1.9 μL) were added and the mixture was stirred at room temperature for 24 hours. After diluting the reaction solution with ethyl acetate, the reaction was quenched with a 1:1 mixed solution of saturated aqueous sodium thiosulfate solution and saturated saline. After separating the organic layer, the aqueous layer was extracted with ethyl acetate. The combined organic layer was washed with saturated saline, dried over anhydrous sodium sulfate, and filtered. The filtrate was concentrated in vacuo and the residue was purified by high performance liquid chromatography to obtain the title compound (138-1: 3.3 mg, trifluoroacetate) and the title compound (138-2: 8.1 mg, trifluoroacetate).

### Example Compound 138-1

¹H-NMR (400 MHz, CDCl₃) δ (ppm): 7.28 (2H, td, J=4.2, 2.4 Hz), 7.01 (2H, d, J=8.3 Hz), 6.74 (1H, d, J=9.3 Hz), 6.49-6.43 (3H, m), 4.38 (2H, dq, J=11.3, 2.9 Hz), 4.05 (2H, dd, J=13.0, 8.2 Hz), 3.83 (3H, s), 3.78 (1H, dd, J=10.0, 2.7 Hz), 3.18 (1H, ddd, J=14.3, 8.4, 5.0 Hz), 3.06 (2H, tt, J=11.3, 4.1 Hz), 2.70 (4H, dd, J=10.0, 9.0 Hz), 2.13 (1H, m), 2.06-1.90 (1H, m), 1.63 (2H, dt, J=35.9, 13.2 Hz), 1.48 (6H, dt, J=32.6, 9.9 Hz), 1.35 (1H, dt, J=22.1, 7.3 Hz), 1.28-1.24 (1H, m), 0.89-0.86 (3H, m).

ESI/MS m/e: 668.1, 670.1 (M⁺+H, C₂₈H₃₈BrF₄N₃O₄S).

### Example Compound 138-2

¹H-NMR (400 MHz, CDCl₃) δ (ppm): 7.30-7.27 (2H, m), 7.01 (2H, t, J=6.1 Hz), 6.75 (1H, d, J=9.3 Hz), 6.46 (3H, td, J=10.3, 2.2 Hz), 4.40 (2H, t, J=5.4 Hz), 4.04 (2H, dq, J=19.0, 5.2 Hz), 3.83 (3H, s), 3.77 (1H, dd, J=9.9, 2.3 Hz), 3.42 (2H, t, J=5.2 Hz), 3.08 (3H, s), 3.04 (1H, t, J=6.1 Hz), 2.71 (1H, dd, J=12.1, 8.2 Hz), 2.02 (2H, dtt, J=53.6, 19.6, 7.3 Hz), 1.59 (1H, ddd, J=19.0, 8.8, 5.0 Hz), 1.47 (6H, dt, J=22.4, 7.9 Hz), 1.35 (1H, dt, J=22.2, 7.3 Hz), 1.26 (1H, t, J=7.1 Hz), 0.88 (3H, t, J=7.4 Hz).

ESI/MS m/e: 684.1, 686.1 (M⁺+H, C₂₈H₃₈BrF₄N₃O₅S).

### Example 139

### Synthesis of (2S)—N-((1S)-1-{[(2,4-dimethoxyphenyl)amino]methyl}-3-(methylsulfinyl)propyl)-2-[((1S)-2,2,2-trifluoro-1-phenylethyl)amino]-4-methylpentanamide (139)

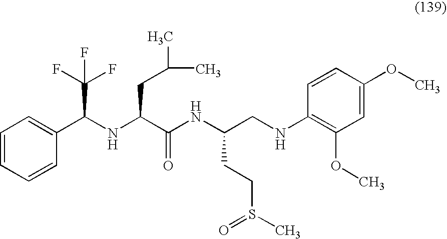

Example Compound 139 was synthesized according to the method described in Example 132, using (2S)—N-((1S)-1-{[(2,4-dimethoxyphenyl)amino]methyl}-3-methylthiopropyl)-2-[((1S)-2,2,2-trifluoro-1-phenylethyl)amino]-4-methylpentanamide as a starting material.

ESI/MS m/e: 558.2 (M⁺+H, C₂₇H₃₈F₃N₃O₄S).

### Example 140

### Synthesis of 2-{4-[((2S)-2-{(2S)-2-[((1S)-2,2,2-trifluoro-1-phenylethyl)amino]-4-fluoro-4-methylpentanoylamino}butyl)amino]-3-methoxyphenoxy}propanoic acid (140)

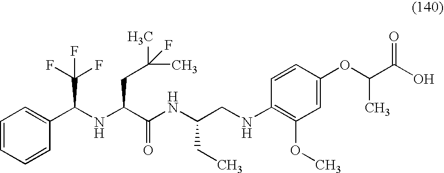

Methyl 2-{4-[((2S)-2-{(2S)-2-[((1S)-2,2,2-trifluoro-1-phenylethyl)amino]-4-fluoro-4-methylpentanoylamino}butyl)amino]-3-methoxyphenoxy}propanoate (21: 65 mg) was dissolved in 1,2-dichloroethane (555 μL). To this solution, trimethyltin hydroxide (50 mg) was added and the mixture was stirred at 60° C. for 3 hours. The reaction solution was concentrated in vacuo and the residue was diluted with ethyl acetate. The organic layer was washed with 1:9 mixed solution of 0.1 mol/L hydrochloric acid and saturated saline, and then with saturated saline. The organic layer was dried over anhydrous sodium sulfate and filtered. The filtrate was concentrated in vacuo and the residue was purified by high performance liquid chromatography to obtain the title compound (140: 65.6 mg, trifluoroacetate).

¹H-NMR (400 MHz, CDCl₃) δ (ppm): 7.30-7.27 (1H, m), 7.23-7.17 (4H, m), 6.81 (1H, d, J=9.3 Hz), 6.50 (1H, d, J=1.7 Hz), 6.42-6.37 (2H, m), 4.66 (1H, q, J=6.7 Hz), 4.08 (1H, q, J=7.2 Hz), 3.99 (1H, tt, J=12.8, 4.5 Hz), 3.80 (1H, dd, J=10.1, 2.3 Hz), 3.77 (3H, s), 2.94 (1H, dd, J=12.3, 5.0 Hz), 2.63 (1H, dd, J=12.3, 7.9 Hz), 2.08 (1H, tt, J=23.8, 7.0 Hz), 1.94 (1H, ddd, J=23.5, 10.9, 4.5 Hz), 1.58 (3H, d, J=6.8 Hz), 1.55-1.40 (7H, m), 1.25 (1H, ddd, J=28.2, 15.4, 7.9 Hz), 0.82 (3H, t, J=7.4 Hz).

ESI/MS m/e: 572.2 (M⁺+H, C₂₈H₃₇F₄N₃O₅).

Hereinafter, the compounds described in Example 141 to Example 156 were synthesized according to the method described in Example 140 or under the general conditions of ester hydrolysis (Reference literature: Protective Groups in Organic Synthesis, Third Edition, John Wiley & Sons, Inc.), using the corresponding starting materials and reagents. Their structures, NMR spectra, and M⁺+H observed by LC/MS, i.e., the measured value observed as the value of the compound molecular weight (M) with proton (H⁺) are summarized in Table 11 below.

TABLE 11

| Example | Compound | | | |
|---|---|---|---|---|
| No. | No. | Structure | M⁺ + H | NMR |
| 141 | 141 | 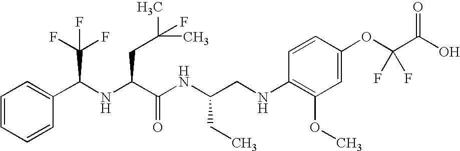 | 594.2 | ¹H-NMR (CDCl₃) δ: 7.32 (6H, s), 7.01 (1H, d, J = 8.5 Hz), 6.74 (2H, dt, J = 10.8, 3.7 Hz), 6.27 (2H, s), 4.20 (1H, q, J = 7.3 Hz), 3.97-3.89 (1H, m), 3.79 (4H, t, J = 6.3 Hz), 3.21 (1H, dd, J = 12.7, 2.4 Hz), 2.80 (1H, dd, J = 12.7, 8.8 Hz), 2.11 (1H, ddd, J = 32.7, 14.9, 2.1 Hz), 1.98- 1.91 (1H, m), 1.53-1.42 (7H, m), 1.26 (1H, ddd, J = 28.7, 14.3, 7.3 Hz), 0.79 (3H, t, J = 7.4 Hz). |
| 142 | 142 | 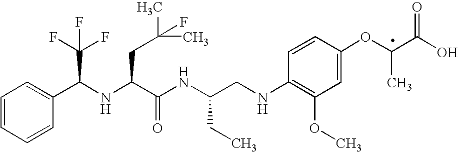 Another enantiomer regarding the asymmetric center (*) at the right end of the compound 140 | 572.2 | ¹H-NMR (CDCl₃) δ: 7.29-7.27 (1H, m), 7.21 (4H, dt, J = 21.9, 6.5 Hz), 6.82 (1H, d, J = 9.3 Hz), 6.50 (1H, s), 6.40 (2H, t, J = 8.7 Hz), 4.64 (1H, q, J = 6.7 Hz), 4.07 (1H, q, J = 7.2 Hz), 3.97 (1H, tt, J = 12.7, 4.4 Hz), 3.81 (4H, m, J = 13.4, 11.5 Hz), 2.95 (1H, dd, J = 12.4, 4.9 Hz), 2.62 (1H, dd, J = 12.4, 7.8 Hz), 2.14-2.01 (1H, m), 1.94 (1H, tt, J = 13.9, 5.4 Hz), 1.56 (3H, d, J = 6.6 Hz), 1.46 (7H, ddd, J = 26.3, 12.6, 6.6 Hz), 1.25 (1H, ddd, J = 33.2, 12.4, 7.0 Hz), 0.82 (3H, t, J = 7.4 Hz). |
| 143 | 143 | 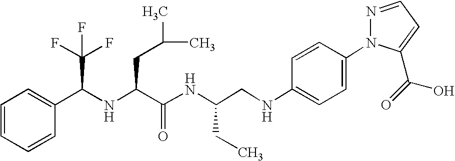 | 546.15 | |
| 144 | 144 | 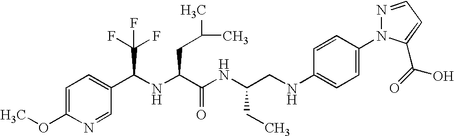 | 577.15 | |
| 145 | 145 | 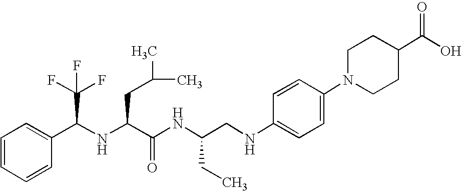 | 563.2 | |
| 146 | 146 | 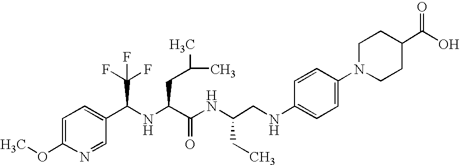 | 594.2 | |
| 147 | 147 | 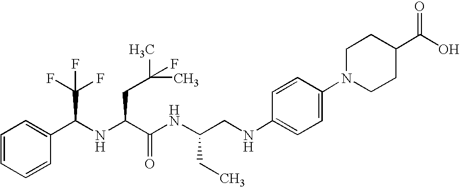 | 581.2 | |
| 148 | 148 | 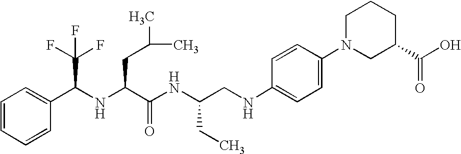 | 563.2 | |
| 149 | 149 | 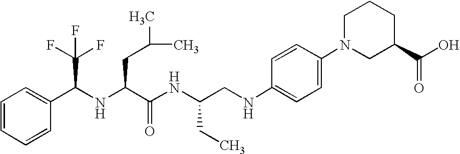 | 563.2 | |
| 150 | 150 | 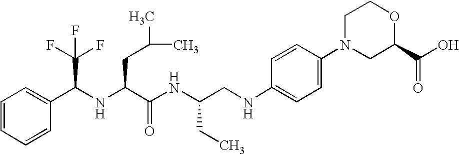 | 564.15 | |
| 151 | 151 | 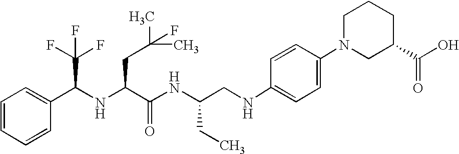 | 581.15 | |
| 152 | 152 | 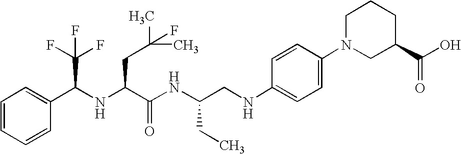 | 581.15 | |
| 153 | 153 | 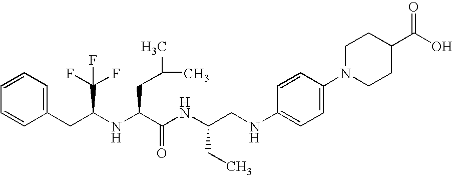 | 577.15 | |
| 154 | 154 | 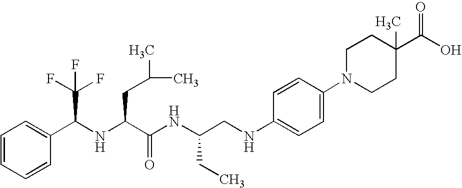 | 577.1 | ¹H-NMR (CD₃OD) δ: 7.84 (1H, d, J = 8.8 Hz), 7.40-7.29 (7H, m), 6.71 (2H, d, J = 9.0 Hz), 4.13 (1H, q, J = 7.6 Hz), 3.84-3.83 (1H, m), 3.57- 3.49 (4H, m), 3.41 (1H, dd, J = 8.3, 5.9 Hz), 3.00 (2H, d, J = 6.6 Hz), 2.43 (2H, d, J = 14.1 Hz), 1.93-1.77 (3H, m), 1.65-1.61 (1H, m), 1.50- 1.28 (7H, m), 0.97-0.82 (10H, m). |
| 155 | 155 | 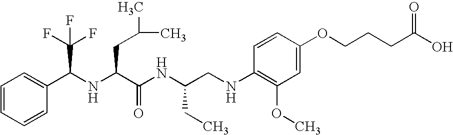 | 568.3 | |
| 156 | 156 | 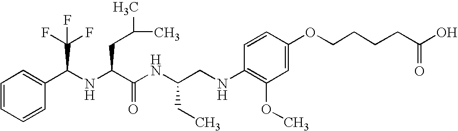 | 582.2 | |

### Example 157

### Synthesis of (2S)—N-((1S)-1-{[(2,4-dimethoxyphenyl)amino]methyl}propyl)-2-{[(1S)-2,2,2-trifluoro-1-(4-hydroxyphenyl)ethyl]amino}-4-methylpentanamide (157)

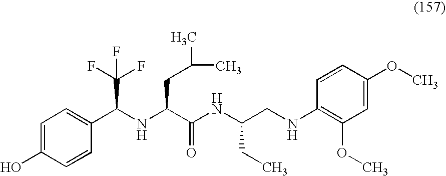

(2S)—N-((1S)-1-{[(2,4-dimethoxyphenyl)amino]methyl}propyl)-2-({(1S)-2,2,2-trifluoro-1-[4-(1,1,2,2-tetramethyl-1-silapropoxy)phenyl]ethyl}amino)-4-methylpentanamide was synthesized according to the method described in Example 1. (2S)—N-((1S)-1-{[(2,4-dimethoxyphenyl)amino]methyl}propyl)-2-({(1S)-2,2,2-trifluoro-1-[4-(1,1,2,2-tetramethyl-1-silapropoxy)phenyl]ethyl}amino)-4-methylpentanamide (42 mg) was dissolved in tetrahydrofuran (1 mL). To this solution, tetrabutylammonium fluoride (1 mol/L, tetrahydrofuran solution, 0.1 mL) was added and the mixture was stirred at room temperature for 1 hour. The reaction solution was concentrated in vacuo and the residue was purified by high performance liquid chromatography to obtain the title compound (157: 1.2 mg, trifluoroacetate).

ESI/MS m/e: 512.2 (M⁺+H, C₂₆H₃₆F₃N₃O₄).

### Example 158

### Synthesis of (2S)—N-((1S)-1-{[(4-morpholin-4-ylphenyl)amino]methyl}propyl)-2-{[(1S)-2,2,2-trifluoro-1-(4-hydroxyphenyl)ethyl]amino}-4-methylpentanamide (158)

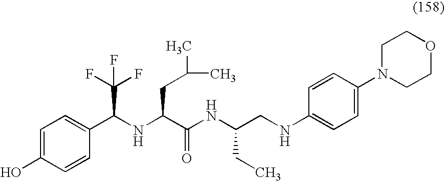

(2S)—N-((1S)-1-{[(4-morpholin-4-ylphenyl)amino]methyl}propyl)-2-{[(1S)-2,2,2-trifluoro-1-(4-hydroxyphenyl)ethyl]amino}-4-methylpentanamide was synthesized according to the method described in Example 129, using (2S)—N-((1S)-1-{[(4-morpholin-4-ylphenyl)amino]methyl}propyl)-2-({(1S)-2,2,2-trifluoro-1-[4-(1,1,2,2-tetramethyl-1-silapropoxy)phenyl]ethyl}amino)-4-methylpentanamide as a starting material.

ESI/MS m/e: 537.2 (M⁺+H, C₂₈H₃₉F₃N₄O₃).

### Example 159

### Synthesis of (5S)-5-{(2S)-2-[((1S)-2,2,2-trifluoro-1-phenylethyl)amino]-4-methylpentanoylamino}-6-[(4-morpholin-4-ylphenyl)amino]hexanoic acid (159)

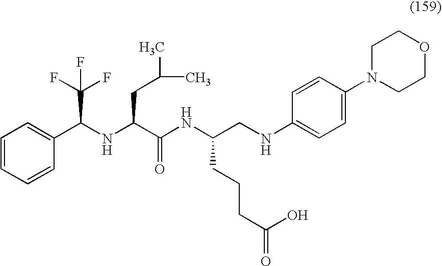

tert-Butyl (5S)-5-{(2S)-2-[((1S)-2,2,2-trifluoro-1-phenylethyl)amino]-4-methylpentanoylamino}-6-[(4-morpholin-4-ylphenyl)amino]hexanoate (93: 25.1 mg) was dissolved in dichloromethane (300 μL). To this solution, hydrogen chloride (4 mol/L, 1,4-dioxane solution, 150 μL) was added and the mixture was stirred at room temperature for 18 hours. The reaction was quenched with neutralizing the mixture with saturated sodium hydrogen carbonate aqueous solution. The organic layer was washed with saturated saline, dried over anhydrous sodium sulfate, and filtered. The filtrate was concentrated in vacuo to obtain the title compound (159: 22.5 mg, free base).

ESI/MS m/e: 579.2 (M⁺+H, C₃₀H₄₁F₃N₄O₄).

### Example 160

### Synthesis of (5S)-5-{(2S)-2-[((1S)-2,2,2-trifluoro-1-phenylethyl)amino]-4-fluoro-4-methylpentanoyl amino}-6-[(4-morpholin-4-ylphenyl)amino]hexanoic acid (160)

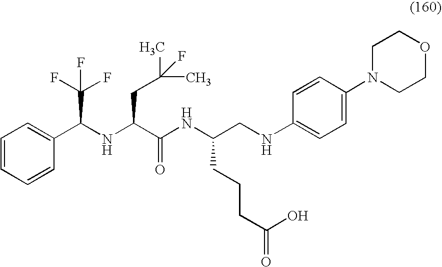

(5S)-5-{(2S)-2-[((1S)-2,2,2-trifluoro-1-phenylethyl)amino]-4-fluoro-4-methylpentanoylamino}-6-[(4-morpholin-4-ylphenyl)amino]hexanoic acid was synthesized according to the method described in Example 150, using tert-butyl (5S)-5-{(2S)-2-[((1S)-2,2,2-trifluoro-1-phenylethyl)amino]-4-fluoro-4-methylpentanoyl amino}-6-[(4-morpholin-4-ylphenyl)amino]hexanoate as a starting material.

ESI/MS m/e: 596.1 (M⁺+H, C₂₈H₃₉F₃N₄O₃).

### Example 161

### Synthesis of (2S)—N-[(1S)-1-({[4-(cyanomethoxy)-2-methoxyphenyl]amino}methyl)propyl]-2-[((1S)-2,2,2-trifluoro-1-phenylethyl)amino]-4-fluoro-4-methylpentanamide (161)

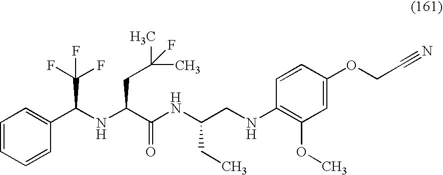

(2S)—N-[(1S)-1-({[4-hydroxy-2-methoxyphenyl]amino}methyl)propyl]-2-[((1S)-2,2,2-trifluoro-1-phenylethyl)amino]-4-fluoro-4-methylpentanamide was synthesized according to the method described in Example 1 and Example 110. Sodium hydride (50 to 72% in mineral oil, 2.2 mg) was suspended in tetrahydrofuran (100 μL). To this suspension, a tetrahydrofuran solution (150 μL) of (2S)—N-[(1S)-1-({[4-hydroxy-2-methoxyphenyl]amino}methyl)propyl]-2-[((1S)-2,2,2-trifluoro-1-phenylethyl)amino]-4-fluoro-4-methylpentanamide (25 mg) was added dropwise, and then N,N-dimethylformamide (250 μL) was added. The mixture was stirred for 30 minutes. After adding bromoacetonitrile (10 μL) dropwise to the reaction solution, the mixture was stirred at room temperature for 30 minutes. The reaction was quenched with a 1:1 mixed solution of saturated aqueous ammonium chloride solution and saturated saline, and extracted with ethyl acetate. The organic layer was washed with saturated saline, dried over anhydrous sodium sulfate, and filtered. The filtrate was concentrated in vacuo and the residue was purified by high performance liquid chromatography to obtain the title compound (161: 9.3 mg, trifluoroacetate).

¹H-NMR (400 MHz, CDCl₃) δ (ppm): 7.34 (6H, ddd, J=13.8, 6.9, 4.1 Hz), 7.25 (1H, d, J=8.5 Hz), 7.12 (1H, d, J=8.5 Hz), 6.57 (2H, dt, J=10.8, 3.7 Hz), 4.76 (2H, s), 4.20 (1H, q, J=7.4 Hz), 4.00-3.93 (1H, m), 3.86 (3H, s), 3.83-3.77 (1H, m), 3.22 (1H, dd, J=12.4, 2.9 Hz), 2.85 (1H, dd, J=12.4, 8.8 Hz), 2.11 (1H, tdd, J=18.7, 10.3, 4.8 Hz), 1.96 (1H, dt, J=22.5, 7.2 Hz), 1.88-1.63 (1H, m), 1.47 (7H, tt, J=14.5, 4.9 Hz), 1.29 (2H, tt, J=22.7, 9.1 Hz), 1.07 (1H, t, J=7.6 Hz), 0.80 (3H, t, J=7.4 Hz).

ESI/MS m/e: 539.2 (M⁺+H, C₂₇H₃₄F₄N₄O₃).

### Example 162

For the compounds synthesized according to the above-mentioned methods, further analysis of high performance liquid chromatography (HPLC) and mass spectrometry using Time Of Flight Mass Spectroscopy (TOF-MS) equipped with an electron spray ion source were performed.

The retention times (unit: minute) of the compounds in HPLC analysis in the analysis conditions described below are shown in Table 12 below as the HPLC retention time.

HPLC measurement conditions
Measurement apparatus: Hewlett-Packard 1100HPLC
Column: Imtakt Cadenza CD-C18 100 mm×4.6 mm, 3 μm
UV: PDA detection (254 nm)
Column temperature: 40° C.
Gradient condition:

Solvent: A: H₂O/acetonitrile=95:5
0.05% TFA (trifluoroacetic acid)

B: H₂O/acetonitrile=5:95
0.05% TFA (trifluoroacetic acid)

Flow rate: 1.0 mL/minute

Gradient: 0 to 1 minute, Solvent B: 10%, Solvent A: 90%
1 to 13 minutes, Solvent B: 10%→70%, Solvent A: 90%→30%
13 to 14 minutes, Solvent B: 70%→100%, Solvent A: 30%→0%
14 to 16 minutes, Solvent B: 100%, Solvent A: 0%
16 to 19 minutes, Solvent B: 100%→10%, Solvent A: 0%→90%

In addition, as for the result of mass spectroscopic analysis, values of “M⁺+H” (obs. Mass, i.e., observed values of molecular weight of the compound (M) plus proton (H⁺)) and calculated values of “M⁺+H” (pred. Mass), along with the molecular formula derived from the value of the observed “M⁺+H” are shown in Table 12 below.

TOF-MS measurement conditions
Mass spectrometry apparatus: Shimadzu Corporation LCMS-IT-TOF

### LC: Prominence

Column: Phenomenex Synergi Hydro-RP 100A 4.0 mm×20 mm, 2 μm
UV: PDA detection (254 nm)
Flow rate: 0.6 mL/minute
Column temperature: 40° C.
Detection voltage: 1.60 kV
Gradient condition:
Solvent A: H₂O/acetonitrile=95:5
   0.05% TFA
B: H₂O/acetonitrile=5:95
   0.05% TFA
Flow rate: 0.5 mL/minute
Gradient: 0 to 0.2 minute, Solvent B: 2%, Solvent A: 98%
   0.2 to 2.5 minutes, Solvent B: 2%→100%, Solvent A: 98%→0%
   2.5 to 3.8 minutes, Solvent B: 100%, Solvent A: 0%
   3.8 to 4.0 minutes, Solvent B: 100%→2%, Solvent A: 0%→98%
   4.0 to 5.0 minutes, Solvent B: 0%, Solvent A: 100%

TABLE 12

| | Synthetic | HPLC | | | molecular |
|---|---|---|---|---|---|
| | Method | Retention | obs Mass | Pred Mass | Formula |
| Compound No. | (Route) | Time (min) | (M⁺ + H) | (M⁺ + H) | (M) |
| 1 | A | 10.07 | 638.2671 | 638.2670 | C₃₂H₃₉F₄N₃O₄S |
| 2 | A | 11.30 | 730.2922 | 730.2932 | C₃₈H₄₃F₄N₃O₅S |
| 3 | A | 9.23 | 640.2467 | 640.2463 | C₃₁H₃₇F₄N₃O₅S |
| 4 | A | 10.87 | 696.3074 | 696.3089 | C₃₅H₄₅F₄N₃O₅S |
| 5 | A | 10.23 | 668.2769 | 668.2776 | C₃₃H₄₁F₄N₃O₅S |
| 6 | A | 11.20 | 592.1797 | 592.1792 | C₂₆H₃₄BrF₄N₃O₃ |
| 7 | G | 10.43 | 514.2684 | 514.2687 | C₂₆H₃₅F₄N₃O₃ |
| 8 | A | 10.78 | 508.2777 | 508.2782 | C₂₇H₃₆F₃N₃O₃ |
| | | 10.89 | | | |
| 9 | A | 9.49 | 693.3079 | 693.3092 | C₃₅H₄₄F₄N₄O₄S |
| 10 | A | 9.82 | 691.3274 | 691.3300 | C₃₆H₄₆F₄N₄O₃S |
| 11 | A | 9.88 | 533.3110 | 533.3098 | C₂₉H₃₉F₃N₄O₂ |
| | | 9.96 | | | |
| 12 | A | 9.44 | 651.2990 | 651.2987 | C₃₃H₄₂F₄N₄O₃S |
| 13 | A | 10.13 | 575.1995 | 575.2003 | C₂₆H₃₅BrF₄N₄O |
| 14 | A | 9.86 | 491.2987 | 491.2992 | C₂₇H₃₇F₃N₄O |
| | | 9.94 | | | |
| 15 | A | 10.87 | 592.1818 | 592.1792 | C₂₆H₃₄BrF₄N₃O₃ |
| 16 | A | 12.13 | 652.1813 | 652.1826 | C₂₈H₃₈BrF₄N₃O₃S |
| 17 | A | 10.97 | 663.1977 | 663.1986 | C₂₉H₃₉BrF₄N₄O₂S |
| 18 | A | 10.66 | 633.1880 | 633.1880 | C₂₈H₃₇BrF₄N₄OS |
| 19 | A | 12.94 | 593.1755 | 593.1745 | C₂₅H₃₃BrF₄N₄O₃ |
| 20 | A | 12.80 | 726.2172 | 726.2160 | C₃₄H₄₀BrF₄N₃O₅ |
| 21 | A | 11.19 | 586.2902 | 586.2899 | C₂₉H₃₉F₄N₃O₅ |
| 22 | A | 12.26 | 614.2650 | 614.2664 | C₂₈H₃₅F₈N₃O₃ |
| 23 | A | 9.80 | 574.2559 | 574.2557 | C₂₇H₃₈F₃N₃O₅S |
| 24 | A | 8.82 | 599.2849 | 599.2873 | C₂₉H₄₁F₃N₄O₄S |
| 25 | A | 9.71 | 527.2836 | 527.2840 | C₂₆H₃₇F₃N₄O₄ |
| 26 | A | 9.23 | 552.3147 | 552.3156 | C₂₈H₄₀F₃N₅O₃ |
| 27 | A | | 522.3057 | 522.3050 | C₂₇H₃₈F₃N₅O₂ |
| 29 | A | 9.75 | 565.2985 | 565.2996 | C₂₉H₃₉F₃N₄O₄ |
| 30 | A | 9.72 | 595.3457 | 595.3466 | C₃₁H₄₅F₃N₄O₄ |
| 31 | A | 7.24 | 538.3004 | 538.3000 | C₂₇H₃₈F₃N₅O₃ |
| 32 | A | 8.53 | 522.3048 | 522.3050 | C₂₇H₃₈F₃N₅O₂ |
| 33 | A | 10.23 | 587.3009 | 587.3015 | C₂₉H₃₉F₅N₄O₃ |
| 34 | A | 10.44 | 555.2703 | 555.2708 | C₂₈H₃₈ClF₃N₄O₂ |
| 35 | A | 8.18 | 564.3534 | 564.3520 | C₃₀H₄₄F₃N₅O₂ |
| 36 | A | 9.99 | 539.3001 | 539.3004 | C₂₈H₃₈F₄N₄O₂ |
| 37 | A | 10.30 | 535.3244 | 535.3254 | C₂₉H₄₁F₃N₄O₂ |
| 38 | A | 9.75 | 619.2778 | 619.2772 | C₂₈H₄₁F₃N₄O₆S |
| 39 | A | 7.48 | 589.2651 | 589.2666 | C₂₇H₃₉F₃N₄O₅S |
| 40 | A | 9.28 | 666.2507 | 666.2489 | C₂₉H₄₂F₃N₃O₇S₂ |
| 41 | A | 7.61 | 605.2606 | 605.2615 | C₂₇H₃₉F₃N₄O₆S |
| 42 | A | 9.36 | 544.2796 | 544.2793 | C₂₇H₃₇F₄N₃O₄ |
| 43 | A | 7.58 | 552.3128 | 552.3156 | C₂₈H₄₀F₃N₅O₃ |
| 44 | A | 8.50 | 527.2842 | 527.2840 | C₂₆H₃₇F₃N₄O₄ |
| 45 | A | 14.52 | 512.2892 | 512.2883 | C₃₀H₃₆F₃N₃O |
| 46 | A | 13.97 | 543.2930 | 543.2941 | C₃₀H₃₇F₃N₄O₂ |
| 47 | A | 12.81 | 502.2794 | 502.2788 | C₂₇H₃₄F₃N₅O |
| 48 | A | 12.06 | 533.2836 | 533.2846 | C₂₇H₃₅F₃N₆O₂ |
| 49 | A | 10.75 | 591.3489 | 591.3517 | C₃₂H₄₅F₃N₄O₃ |
| 50 | A | 10.15 | 622.3573 | 622.3575 | C₃₂H₄₆F₃N₅O₄ |
| 51 | A | 10.64 | 549.3406 | 649.3411 | C₃₀H₄₃F₃N₄O₂ |
| 52 | A | 9.97 | 580.3467 | 580.3469 | C₃₀H₄₄F₃N₅O₃ |
| 53 | A | 11.30 | 519.2934 | 519.2941 | C₂₈H₃₇F₃N₄O₂ |
| 54 | A | 10.54 | 550.2990 | 550.3000 | C₂₈H₃₈F₃N₅O₃ |
| 55 | A | 10.46 | 609.3394 | 609.3422 | C₃₂H₄₄F₄N₄O₃ |
| 56 | A | 10.47 | 579.3143 | 579.3153 | C₃₀H₄₁F₃N₄O₄ |
| 57 | A | 10.83 | 530.2630 | 530.2625 | C₂₉H₃₄F₃N₃O₃ |
| 58 | A | 9.85 | 555.2930 | 555.2941 | C₃₁H₃₇F₃N₄O₂ |
| 59 | A | 11.39 | 510.2933 | 510.2938 | C₂₇H₃₈F₃N₃O₃ |
| 60 | A | 11.08 | 496.2780 | 496.2782 | C₂₆H₃₆F₃N₃O₃ |
| 61 | A | 11.67 | 522.2936 | 522.2938 | C₂₈H₃₈F₃N₃O₃ |
| 28 | A | 10.47 | 494.2625 | 494.2625 | C₂₆H₃₄F₃N₃O₃ |
| 62 | A | 10.74 | 536.2191 | 536.2189 | C₂₇H₃₂F₃N₃O₃S |
| 63 | A | 11.72 | 558.2937 | 558.2938 | C₃₁H₃₈F₃N₃O₃ |
| 64 | A | 11.85 | 524.3096 | 524.3095 | C₂₈H₄₀F₃N₃O₃ |
| 65 | A | 11.78 | 554.3194 | 554.3200 | C₂₉H₄₂F₃N₃O₄ |
| 66 | A | 11.44 | 542.2648 | 542.2659 | C₂₇H₃₈F₃N₃O₃S |
| 67 | A | 11.39 | 572.2769 | 572.2764 | C₂₈H₄₀F₃N₃O₄S |
| 68 | A | 11.01 | 655.3453 | 655.3466 | C₃₆H₄₅F₃N₄O₄ |
| 69 | A | 10.86 | 641.3299 | 641.3309 | C₃₅H₄₃F₃N₄O₄ |
| 70 | A | 10.54 | 482.2619 | 482.2625 | C₂₅H₃₄F₃N₃O₃ |
| 71 | A | 9.45 | 507.2936 | 507.2941 | C₂₇H₃₇F₃N₄O₂ |
| 72 | A | 6.86 | 524.2852 | 524.2843 | C₂₆H₃₆F₃N₅O₃ |
| 73 | A | 8.42 | 585.2719 | 585.2717 | C₂₈H₃₉F₃N₄O₄S |
| 74 | A | 6.42 | 508.2889 | 508.2894 | C₂₆H₃₆F₃N₅O₂ |
| 75 | A | 8.74 | 538.2975 | 538.3000 | C₂₇H₃₈F₃N₅O₃ |
| 76 | A | 7.83 | 499.2516 | 499.2527 | C₂₄H₃₃F₃N₄O₄ |
| 77 | A | 9.43 | 560.2394 | 560.2401 | C₂₆H₃₆F₃N₃O₅S |
| 78 | A | 7.58 | 483.2578 | 483.2578 | C₂₄H₃₃F₃N₄O₃ |
| 79 | A | 9.88 | 513.2677 | 513.2683 | C₂₅H₃₅F₃N₄O₄ |
| 80 | A | 10.44 | 574.2550 | 574.2557 | C₂₇H₃₈F₃N₃O₅S |
| 81 | A | 7.86 | 591.2443 | 591.2459 | C₂₆H₃₇F₃N₄O₆S |
| 82 | A | 9.49 | 652.2322 | 652.2333 | C₂₈H₄₀F₃N₃O₇S₂ |
| 83 | A | 7.70 | 575.2490 | 575.2510 | C₂₆H₃₇F₃N₄O₅S |
| 84 | A | 9.93 | 605.2614 | 605.2615 | C₂₇H₃₉F₃N₄O₆S |
| 85 | A | 11.34 | 582.3142 | 582.3149 | C₃₀H₄₂F₃N₃O₅ |
| 86 | A | 12.09 | 610.3466 | 610.3462 | C₃₂H₄₆F₃N₃O₅ |
| 87 | A | 11.07 | 627.3498 | 627.3517 | C₃₅H₄₅F₃N₄O₃ |
| 88 | A | 14.03 | 521.3095 | 521.3098 | C₂₈H₃₉F₃N₄O₂ |
| 89 | A | 10.50 | 583.3095 | 583.3102 | C₂₉H₄₁F₃N₄O₅ |
| 90 | A | 12.55 | 534.2546 | 534.2550 | C₂₆H₃₃F₆N₃O₂ |
| 91 | A | 11.22 | 635.3772 | 635.3779 | C3₄H₄₉F₃N₄O₄ |
| 92 | A | 9.35 | 546.3030 | 546.3050 | C₂₉H₃₈F₃N₅O₂ |
| 93 | A | 9.44 | 532.2900 | 532.2894 | C₂₈H₃₆F₃N₅O₂ |
| 94 | A | 9.02 | 564.2947 | 564.2956 | C₂₉H₃₇F₄N₅O₂ |
| 95 | A | 10.91 | 526.2882 | 526.2887 | C₂₇H₃₈F₃N₃O₄ |
| 96 | A | 12.36 | 542.2995 | 542.2989 | C₃₁H₃₈F₃N₃O₂ |
| 97 | A | 12.31 | 572.3101 | 572.3095 | C₃₂H₄₀F₃N₃O₃ |
| 98 | A | 11.04 | 556.2997 | 556.2993 | C₂₈H₄₀F₃N₃O₅ |
| 99 | A | 11.41 | 480.2458 | 480.2469 | C₂₅H₃₂F₃N₃O₃ |
| 100 | A | 11.34 | 510.2582 | 510.2574 | C₂₆H₃₄F₃N₃O₄ |
| 101 | A | 7.47 | 592.3589 | 592.3581 | C₃₀H₄₄F₃N₇O₂ |
| 102 | A | 10.85 | 698.3867 | 698.3888 | C₃₈H₅₀F₃N₅O₄ |
| 103 | A | 10.62 | 684.3728 | 684.3731 | C₃₇H₄₈F₃N₅O₄ |
| 104 | A | 10.54 | 656.3420 | 656.3418 | C₃₅H₄₄F₃N₅O₄ |
| 105 | A | 10.68 | 670.3564 | 670.3575 | C₃₆H₄₆F₃N₅O₄ |
| 106 | A | 7.49 | 550.3346 | 550.3363 | C₂₉H₄₂F₃N₅O₂ |
| 107 | A | 7.19 | 568.3254 | 568.3269 | C₂₉H₄₁F₄N₅O₂ |
| 108 | A | 9.45 | 535.3263 | 535.3254 | C₂₉H₄₁N₄O₂F₃ |
| 109 | A | 11.65 | 633.3998 | 633.3986 | C₃₅H₅₁N₄O₃F₃ |
| 110 | A | 10.46 | 494.2625 | 494.2625 | C₂₆H₃₄F₃N₃O₃ |
| | | 10.53 | | | |
| 111 | A | 14.01 | 622.2717 | 622.2710 | C₂₉H₃₇F₆N₃O₅ |
| 112 | A | 9.92 | 502.2778 | 502.2788 | C₂₇H₃₄F₃N₅O |
| 113 | A | 9.24 | 533.2841 | 533.2846 | C₂₇H₃₅F₃N₆O₂ |
| 114 | A | 13.72 | 574.3006 | 574.3000 | C₃₀H₃₈F₃N₅O₃ |
| 115 | A | 13.05 | 605.3083 | 605.3098 | C₃₀H₃₉F₃N₆O₄ |
| 116 | A | 9.90 | 526.2551 | 526.2557 | C₂₃H₃₈F₃N₃O₅S |
| 117 | G | 9.62 | 558.2578 | 558.2586 | C₂₇H₃₅F₄N₃O₅ |
| 118 | A | 10.12 | 584.2743 | 584.2742 | C₂₉H₃₇F₄N₃O₅ |
| 119 | A | 10.01 | 540.2675 | 540.2680 | C₂₇H₃₆F₃N₃O₅ |
| 120 | A | 9.39 | 571.2741 | 571.2738 | C₂₇H₃₇F₃N₄O₆ |
| 121 | A | 7.19 | 541.2613 | 541.2632 | C₂₆H₃₅F₃N₄O₅ |
| 122 | A | 8.98 | 618.2450 | 618.2455 | C₂₈H₃₈F₃N₃O₇S |
| 123 | A | 7.38 | 557.2575 | 557.2581 | C₂₆H₃₅F₃N₄O₆ |
| 124 | A | 10.85 | 580.2976 | 580.2993 | C₃₀H₄₀F₃N₃O₅ |
| 125 | A | 8.62 | 551.3179 | 551.3204 | C₂₉H₄₁F₃N₄O₃ |
| 126 | A | 7.38 | 564.3529 | 564.3520 | C₃₀H₄₄F₃N₅O₂ |
| 127 | A | 7.27 | 550.3369 | 550.3363 | C₂₉H₄₂F₃N₅O₂ |
| 128 | A | 7.27 | 522.3048 | 522.3050 | C₂₇H₃₈F₃N₅O₂ |
| 129 | A | 7.23 | 536.3188 | 536.3207 | C₂₈H₄₀F₃N₅O₂ |
| 130 | A | 6.88 | 554.3111 | 554.3113 | C₂₈H₃₉F₄N₅O₂ |
| 131 | A | 10.65 | 586.2908 | 586.2899 | C₂₉H₃₉F₄N₃O₅ |
| 132 | A | 11.34 | 582.3160 | 582.3149 | C₃₀H₄₂F₃N₃O₅ |
| 133 | A | 9.20 | 557.2744 | 557.2745 | C₂₇H₃₆F₄N₄O₄ |
| 134 | A | 9.62 | 585.3027 | 585.3058 | C₂₉H₄₀F₄N₄O₄ |
| 135 | A | 9.99 | 611.3199 | 611.3215 | C₃₁H₄₂F₄N₄O₄ |
| 136 | A | 9.64 | 627.3130 | 627.3164 | C₃₁H₄₂F₄N₄O₅ |
| 137 | A | 8.97 | 601.3008 | 601.3008 | C₂₉H₄₀F₄N₄O₅ |
| 138-1 | A | 9.93 | 668.1773 | 668.1775 | C₂₈H₃₈BrF₄N₃O₄S |
| 138-2 | A | 10.73 | 684.1722 | 684.1724 | C₂₈H₃₈BrF₄N₃O₅S |
| 139 | A | 9.58 | 558.2613 | 558.2608 | C₂₇H₃₈F₃N₃O₄S |
| 140 | A | 9.98 | 572.2741 | 572.2742 | C₂₈H₃₇F₄N₃O₅ |
| 141 | A | 11.32 | 594.2384 | 594.2397 | C₂₇H₃₃F₆N₃O₅ |
| 142 | A | 9.97 | 572.2765 | 572.2742 | C₂₈H₃₇F₄N₃O₅ |
| 143 | A | 11.72 | 546.2628 | 546.2614 | C₂₈H₃₄F₃N₅O₃ |
| 144 | A | 10.97 | 577.2736 | 577.2745 | C₂₈H₃₅F₃N₆O₄ |
| 145 | A | 9.83 | 563.3200 | 563.3204 | C₃₀H₄₁F₃N₄O₃ |
| 146 | A | 9.18 | 594.3265 | 594.3262 | C₃₀H₄₂F₃N₅O₄ |
| 147 | A | 9.53 | 581.3119 | 581.3109 | C₃₀H₄₀F₄N₄O₃ |
| 148 | A | 9.86 | 563.3193 | 563.3204 | C₃₀H₄₁F₃N₄O₃ |
| 149 | A | 9.86 | 563.3197 | 563.3204 | C₃₀H₄₁F₃N₄O₃ |
| 150 | A | 9.56 | 565.2973 | 565.2996 | C₂₉H₃₉F₃N₄O₄ |
| 151 | A | 9.56 | 581.3090 | 581.3109 | C₃₀H₄₀F₄N₄O₃ |
| 152 | A | 9.57 | 581.3116 | 581.3109 | C₃₀H₄₀F₄N₄O₃ |
| 153 | A | 10.25 | 577.3355 | 577.3360 | C₃₁H₄₃F₃N₄O₃ |
| 154 | A | 9.97 | 577.3369 | 577.3360 | C₃₁H₄₃F₃N₄O₃ |
| 155 | A | 10.18 | 568.2991 | 568.2993 | C₂₉H₄₀F₃N₃O₅ |
| 156 | A | 10.39 | 582.3145 | 582.3149 | C₃₀H₄₂F₃N₃O₅ |
| 157 | A | 9.71 | 512.2723 | 512.2731 | C₂₆H₃₆F₃N₃O₄ |
| 158 | A | 8.78 | 537.3052 | 537.3047 | C₂₈H₃₉F₃N₄O₃ |
| 159 | A | 8.83 | 579.3140 | 579.3153 | C₃₀H₄₁F₃N₄O₄ |
| 160 | A | 8.52 | 597.3072 | 597.3058 | C₃₀H₄₀F₄N₄O₄ |
| 161 | A | 11.37 | 539.2652 | 539.2640 | C₂₇H₃₄F₄N₄O₃ |

### Example 163

Cathepsin K inhibitory activities of the compounds synthesized according to the methods of the above examples were measured.

Cathepsin K used for evaluation of inhibitory activity was transiently expressed in an animal cell HEK293T (made by GenHunter Corporation) and the active enzyme was obtained as the enzyme fraction by using detergent containing lysis buffer.

The enzyme solution A was prepared at 2.1 times final concentration by diluting the enzyme fraction with assay buffer (50 mM sodium acetate, 50 mM sodium chloride, 2 mM DTT, pH 5.5). The test compound solutions B were prepared at 50 times final target concentrations by dimethylsulfoxide (DMSO). As a substrate solution C, a solution of a fluorescent substrate, benzyloxycarbonyl-L-leucyl-L-arginyl-4-methyl-coumaryl-7-amide (Z-Leu-Arg-MCA (Peptide Institute Inc.), was prepared at 10 μM by an assay buffer.

To the enzyme solution A (38.4 μL) were added the test compound solutions B (1.6 μL) and mixed individually. The mixtures were incubated at room temperature for 15 minutes. To the incubated solutions were added the substrate solution C (40 μL) and the mixtures were reacted at room temperature for 30 minutes respectively. The fluorescence intensities of the enzyme reaction solutions were measured at excitation wavelength of 355 nm and measurement wavelength of 460 nm and the enzyme activities were calculated from these fluorescence intensities caused by 7-amino-4-methylcoumarine released. The enzyme activity with using DMSO instead of the test compound solution B was taken as 100% and the inhibitory rates at each concentration of the test compound were calculated. The volume response curve was fitted to the plots. The 50% inhibitory concentration against cathepsin K was calculated from this curve.

The results are shown in Table 13. Note that the symbols (+, ++, and +++) in this table represent the inhibitory activity values as below. Here, pIC₅₀ is the value representing a negative logarithm of IC₅₀, (−log₁₀(IC₅₀)). IC₅₀ is a 50% inhibitory concentration.

5.0≦pIC₅₀<7.5: +

7.5≦pIC₅₀<8.5: ++

8.5≦pIC₅₀: +++

TABLE 13

| Compound | Activity |
|---|---|
| No. | Intensity |
| 1 | ++ |
| 2 | + |
| 3 | ++ |
| 4 | +++ |
| 5 | +++ |
| 6 | +++ |
| 7 | +++ |
| 8 | ++ |
| 9 | +++ |
| 10 | +++ |
| 11 | ++ |
| 12 | +++ |
| 13 | +++ |
| 14 | + |
| 15 | ++ |
| 16 | +++ |
| 17 | +++ |
| 18 | +++ |
| 19 | ++ |
| 20 | +++ |
| 21 | ++ |
| 22 | ++ |
| 23 | +++ |
| 24 | +++ |
| 25 | ++ |
| 26 | +++ |
| 27 | +++ |
| 28 | + |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | ++ |
| 39 | ++ |
| 40 | ++ |
| 41 | +++ |
| 42 | +++ |
| 43 | +++ |
| 44 | ++ |
| 45 | + |
| 46 | + |
| 47 | + |
| 48 | + |
| 49 | +++ |
| 50 | +++ |
| 51 | +++ |
| 52 | +++ |
| 53 | ++ |
| 54 | ++ |
| 55 | +++ |
| 56 | +++ |
| 57 | + |
| 58 | + |
| 59 | +++ |
| 60 | ++ |
| 61 | + |
| 62 | + |
| 63 | + |
| 64 | +++ |
| 65 | +++ |
| 66 | ++ |
| 67 | ++ |
| 68 | +++ |
| 69 | +++ |
| 70 | +++ |
| 71 | +++ |
| 72 | +++ |
| 73 | +++ |
| 74 | +++ |
| 75 | +++ |
| 76 | ++ |
| 77 | ++ |
| 78 | ++ |
| 79 | ++ |
| 80 | ++ |
| 81 | + |
| 82 | ++ |
| 83 | ++ |
| 84 | + |
| 85 | +++ |
| 86 | +++ |
| 87 | +++ |
| 88 | + |
| 89 | +++ |
| 90 | + |
| 91 | +++ |
| 92 | +++ |
| 93 | ++ |
| 94 | +++ |
| 95 | +++ |
| 96 | ++ |
| 97 | ++ |
| 98 | ++ |
| 99 | + |
| 100 | + |
| 101 | +++ |
| 102 | +++ |
| 103 | +++ |
| 104 | +++ |
| 105 | +++ |
| 106 | +++ |
| 107 | +++ |
| 108 | +++ |
| 109 | +++ |
| 110 | + |
| 111 | + |
| 112 | + |
| 113 | + |
| 114 | + |
| 115 | + |
| 116 | + |
| 117 | +++ |
| 118 | +++ |
| 119 | +++ |
| 120 | +++ |
| 121 | ++ |
| 122 | +++ |
| 123 | ++ |
| 124 | ++ |
| 125 | + |
| 126 | +++ |
| 127 | ++ |
| 128 | ++ |
| 129 | ++ |
| 130 | ++ |
| 131 | ++ |
| 132 | ++ |
| 133 | +++ |
| 134 | +++ |
| 135 | +++ |
| 136 | +++ |
| 137 | +++ |
| 138-1 | +++ |
| 138-2 | +++ |
| 139 | + |
| 140 | ++ |
| 141 | + |
| 142 | ++ |
| 143 | + |
| 144 | + |
| 145 | +++ |
| 146 | +++ |
| 147 | +++ |
| 148 | +++ |
| 149 | +++ |
| 150 | +++ |
| 151 | +++ |
| 152 | +++ |
| 153 | ++ |
| 154 | +++ |
| 155 | +++ |
| 156 | +++ |
| 157 | ++ |
| 158 | +++ |
| 159 | +++ |
| 160 | +++ |
| 161 | +++ |

### Example 164

For the compounds synthesized according to the method of the above Examples and the compounds of formula (B) (the compounds disclosed in WO2002/070517), the metabolic stability test using the human liver microsome was performed and the residual rate of each compound was calculated.

To a human liver microsome solution (950 μL) was added a test compound solution (10 μL, 100 μM, acetonitrile solution) on an ice bath and the solution was divided into two equal parts, solution A and solution B. Note that the composition of the human liver microsome solution was as follows.

20 mg/mL protein human liver microsome (Xenotech LLC Lenexa, US): 10 μL
500 mM potassium phosphate buffer solution (pH 7.4): 200 μL
10 mM EDTA solution: 100 μL
60 mM MgCl₂ solution: 50 μL
100 mM glucose-6-phosphate solution: 50 μL
100 I.U./mL glucose-6-phosphate dehydrogenase solution: 10 μL
purified water: 530 μL

To the solution A (480 μL) was added acetonitrile (500 μL) on an ice bath, and then 25 mM NADPH solution (20 μL) was added. After vortexing, the mixture was centrifuged (3,000 rpm) at 4° C. for 10 minutes, and the supernatant was taken as the sample at the reaction time of 0 minute.

To the solution B (480 μL) was added 25 mM NADPH solution (20 μL). The mixture was incubated at 37° C. for 25 minutes. The reaction was quenched with acetonitrile (500 μL) and vortexing. The mixture was centrifuged (3,000 rpm) at 4° C. for 10 minutes, and the supernatant was taken as the sample at the reaction time of 25 minutes.

LC/MS measurement was performed for the samples at the reaction time of 0 minute and the reaction time of 25 minutes. Based on the peak area of the target molecular weight in the MS measurement, the residual rate of the sample at the reaction time of 25 minutes to the sample at the reaction time of 0 minute was calculated in percentage. The results are shown in Table 14.

TABLE 14

| Compound | Residual Rate at |
|---|---|
| No. | 25 minutes (%) |
| B | 0 |
| 5 | 0 |
| 7 | 0 |
| 31 | 77 |
| 36 | 0 |
| 43 | 46 |
| 46 | 65 |
| 81 | 41 |
| 93 | 63 |
| 101 | 67 |
| 107 | 50 |
| 117 | 111 |
| 118 | 76 |
| 120 | 108 |
| 121 | 105 |
| 124 | 87 |
| 126 | 90 |
| 127 | 87 |
| 129 | 75 |
| 130 | 85 |
| 131 | 123 |
| 140 | 101 |
| 141 | 70 |
| 142 | 96 |
| 145 | 88 |
| 147 | 80 |
| 148 | 85 |
| 149 | 87 |
| 150 | 96 |
| 151 | 90 |
| 152 | 88 |
| 153 | 53 |
| 154 | 74 |
| 155 | 31 |
| 159 | 100 |
| 160 | 90 |

Based on the above, it was shown that the compounds represented by formula (1) or formula (1A) of the present invention tends to be excellent in metabolic stability when at least one of R¹, the substituent of R¹, the substituent of R² selected from the substituent group 2, R⁵, and the substituent of R⁵ represents —COOH or cyano, when the substituent of R² selected from the substituent group 2 represents —N(R^{6a})(R^{6b}) or —N(R^{6a})C(═NR^{6b})(NR^{6c}), or when Ar² has heteroaryl.

### INDUSTRIAL APPLICABILITY

The compound represented by the above-mentioned formula (1) of the present invention and the pharmaceutically acceptable salt thereof have a cysteine protease inhibitory effect (especially cathepsin K inhibitory effect) and can be used as a drug clinically applicable as a cysteine protease inhibitor for treatment or prevention of a disease selected from the group consisting of osteoporosis, osteoarthritis, chronic rheumatoid arthritis, Paget's disease of bone, hypercalcemia, bone metastasis of cancer, and ostealgia.

1. A compound represented by formula (1), or a pharmaceutically acceptable salt thereof: 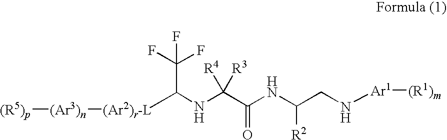
wherein
Ar¹ represents C₆-C₁₀ aryl, or heteroaryl;
R¹ represents a substituent selected from the substituent group 1;
m represents an integer of 0 to 3;
R² represents C₁-C₆ alkyl that may be substituted with the same or different 1 to 6 group(s) selected from the substituent group 2;
R³ and R⁴ are the same or different from each other and represent hydrogen atom or C₁-C₆ alkyl, C₃-C₇ cycloalkyl, C₄-C₉ (cycloalkyl)alkyl, phenyl, heteroaryl, C₇-C₉ phenylalkyl, or C₁-C₃ alkyl substituted with heteroaryl, these substituents may be substituted with the same or different 1 to 6 group(s) selected from the substituent group 3;
when both of R³ and R⁴ are C₁-C₆ alkyl that may be substituted with the same or different 1 to 6 group(s) selected from the substituent group 3, the R³ and R⁴ may bond each other via a single bond, —O—, —NR⁹—, or —S(O)_{q}— to form 3- to 7-membered ring structure containing the carbon atoms to which R³ and R⁴ are bonding;
when R³ and R⁴ do not bond to form a ring structure, either R³ or R⁴ represents a group which is not a hydrogen atom;
L represents a single bond or —(CR¹⁰R¹¹)ₛ—;
s represents any one integer of 1 to 4;
Ar² represents C₆-C₁₀ aryl or heteroaryl;
r represents 0 or 1;
Ar³ represents C₆-C₁₀ aryl or heteroaryl;
n represents 0 or 1;
R⁵ represents a substituent selected from the substituent group 1;
p represents an integer of 0 to 5;
the substituent group 1 represents a group consisting of hydrogen atom, halogen atom, cyano, nitro, —R^{6a}, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)₂N(R^{6a})(R^{6b}), —NR^{6a}S(O)₂R^{6b}, —S(O)_{q}R^{6a}, and —Si(R⁸)₃;
the substituent group 2 represents a group consisting of halogen atom, cyano, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)_{q}R^{6a}, —N(R^{6a})C(═NR^{6b})(NR^{6c}), C₃-C₇ cycloalkyl that may be substituted with R⁷, phenyl that may be substituted with R⁷, and heteroaryl that may be substituted with R⁷;
the substituent group 3 represents halogen atom, hydroxyl, and C₁-C₆ alkoxy, C₁-C₆ alkylthio, C₁-C₆ alkylsulfinyl, and C₁-C₆ alkylsulfonyl group, these substituents may be substituted with halogen atom;
R^{6a}, R^{6b}, and R^{6c} are the same or different from each other and represent hydrogen atom, C₁-C₆ alkyl that may be substituted with R⁷, C₂-C₆ alkenyl that may be substituted with R⁷, C₂-C₆ alkynyl that may be substituted with R⁷, C₃-C₇ cycloalkyl that may be substituted with R⁷, heterocyclyl that may be substituted with R⁷, phenyl that may be substituted with R⁷, heteroaryl that may be substituted with R⁷, C₇-C₁₃ aralkyl that may be substituted with R⁷, C₁-C₃ alkyl substituted with heterocyclyl that may be substituted with R⁷, or C₁-C₃ alkyl substituted with heteroaryl that may be substituted with R⁷; in each substituent in the substituent groups 1 and 2, the R^{6a} and R^{6b}, R^{6a} and R^{6c}, or R^{6b} and R^{6c} may bond each other via a single bond, —O—, —NR⁹—, or —S(O)_{q}— to form 3- to 7-membered ring structure, when R^{6a} and R^{6b}, R^{6a} and R^{6c}, or R^{6b} and R^{6c} existing in one substituent are C₁-C₆ alkyl optionally substituted with R⁷;
q represents an integer of 0 to 2;
R⁷ represents halogen atom, hydroxyl, carboxyl, C₁-C₄ alkyl, C₁-C₄ alkoxy, C₁-C₄ alkoxycarbonyl, C₁-C₄ alkylsulfonyl, C₁-C₄ alkylsulfinyl, or cyano; and
R⁸ represents C₁-C₆ alkyl that may be substituted with R⁷; and
R⁹, R¹⁰, and R¹¹ are the same or different from each other and represent hydrogen atom or C₁-C₆ alkyl that may be substituted with R⁷.

2. The compound according to claim 1 and represented by formula (1A), or a pharmaceutically acceptable salt thereof: 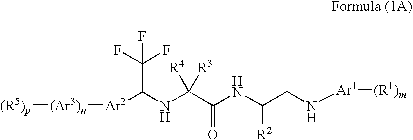
wherein
Ar¹ represents C₆-C₁₀ aryl, or heteroaryl;
R¹ represents a substituent selected from the substituent group 1;
m represents an integer of 0 to 3;
R² represents C₁-C₆ alkyl that may be substituted with the same or different 1 to 6 group(s) selected from the substituent group 2;
R³ and R⁴ are the same or different from each other and represent hydrogen atom or C₁-C₆ alkyl, C₃-C₇ cycloalkyl, C₄-C₉ (cycloalkyl)alkyl, phenyl, heteroaryl, C₇-C₉ phenylalkyl, or C₁-C₃ alkyl substituted with heteroaryl, these substituents may be substituted with the same or different 1 to 6 group(s) selected from the substituent group 3;
when both of R³ and R⁴ are C₁-C₆ alkyl that may be substituted with the same or different 1 to 6 group(s) selected from the substituent group 3, the R³ and R⁴ may bond each other via a single bond, —O—, —NR⁹—, or —S(O)_{q}— to form 3- to 7-membered ring structure containing the carbon atoms to which R³ and R⁴ are bonding;
when R³ and R⁴ do not bond to form a ring structure, either R³ or R⁴ represents a group which is not a hydrogen atom;
Ar² represents C₆-C₁₀ aryl or heteroaryl;
Ar³ represents C₆-C₁₀ aryl or heteroaryl;
n represents 0 or 1;
R⁵ represents a substituent selected from the substituent group 1;
p represents an integer of 0 to 5;
the substituent group 1 represents a group consisting of halogen atom, cyano, nitro, —R^{6a}, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)₂N(R^{6a})(R^{6b}), —NR^{6a}S(O)₂R^{6b}, —S(O)_{q}R^{6a}, and —Si(R⁸)₃;
the substituent group 2 represents a group consisting of halogen atom, cyano, —OR^{6a}, —O(CO)R^{6a}, —COOR^{6a}, —CON(R^{6a})(R^{6b}), —N(R^{6a})(R^{6b}), —NR^{6a}(CO)R^{6b}, —NR^{6a}(CO)N(R^{6b})(R^{6c}), —S(O)_{q}R^{6a}, C₃-C₇ cycloalkyl that may be substituted with R⁷, phenyl that may be substituted with R⁷, and heteroaryl that may be substituted with R⁷;
the substituent group 3 represents halogen atom, hydroxyl, and a C₁-C₆ alkoxy, C₁-C₆ alkylthio, C₁-C₆ alkylsulfinyl, and C₁-C₆ alkylsulfonyl, these substituents may be substituted with halogen atom;
R^{6a}, R^{6b}, and R^{6c} are the same or different from each other and represent hydrogen atom, C₁-C₆ alkyl that may be substituted with R⁷, C₂-C₆ alkenyl that may be substituted with R⁷, C₂-C₆ alkynyl that may be substituted with R⁷, C₃-C₇ cycloalkyl that may be substituted with R⁷, heterocyclyl that may be substituted with R⁷, phenyl that may be substituted with R⁷, heteroaryl that may be substituted with R⁷, C₇-C₃ aralkyl that may be substituted with R⁷, C₁-C₃ alkyl substituted with heterocyclyl that may be substituted with R⁷, or C₁-C₃ alkyl substituted with heteroaryl that may be substituted with R⁷;
in each substituent in the substituent groups 1 and 2, the R^{6a} and R^{6b}, R^{6a} and R^{6c}, or R^{6b} and R^{6c} may bond each other via a single bond, —O—, —NR⁹—, or —S(O)_{q}— to form 3- to 7-membered ring structure, when R^{6a} and R^{6b}, R^{6a} and R^{6c}, or R^{6b} and R^{6c} existing in one substituent are C₁-C₆ alkyl optionally substituted with R⁷;
q represents an integer of 0 to 2;
R⁷ represents halogen atom, hydroxyl, carboxyl, C₁-C₄ alkyl, C₁-C₄ alkoxy, C₁-C₄ alkoxycarbonyl, C₁-C₄ alkylsulfonyl, or C₁-C₄ alkylsulfinyl; and
R⁸ and R⁹ are the same or different from each other and represent C₁-C₆ alkyl that may be substituted with R⁷.

3. The compound according to claim 1 or 2, or a pharmaceutically acceptable salt thereof,
wherein R³ represents C₁-C₆ alkyl, C₃-C₇ cycloalkyl, or C₄-C₉ (cycloalkyl)alkyl, these substituents may be substituted with 1 to 6 fluorine atom(s); and
R⁴ represents hydrogen atom.

4. The compound according to claim 1 or 2, or a pharmaceutically acceptable salt thereof,
wherein R³ represents isobutyl that may be substituted with 1 to 6 fluorine atom(s); and
R⁴ represents hydrogen atom.

5. The compound according to claim 1 or 2, or a pharmaceutically acceptable salt thereof,
wherein R³ and R⁴ form cyclohexane ring containing the carbon atoms to which R³ and R⁴ are bonding.

6. The compound according to any of claims 1 to 5, or a pharmaceutically acceptable salt thereof,
wherein Ar¹ represents C₆-C₁₀ aryl.

7. The compound according to any of claims 1 to 6, or a pharmaceutically acceptable salt thereof,
wherein m represents an integer of 1 to 3.

8. The compound according to claim 7, or a pharmaceutically acceptable salt thereof,
wherein at least one R¹ represents —OR^{6a} or —N(R^{6a})(R^{6b}).

9. The compound according to any of claims 1 to 5, or a pharmaceutically acceptable salt thereof,
wherein —Ar¹—(R¹)ₘ is a substituent represented by formula (2): 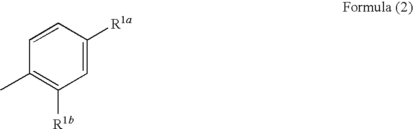
wherein R^{1a} represents —OR^{6a} or —N(R^{6a})(R^{6b}); and
R^{1b} represents halogen atom, —R^{6a}, —OR^{6a}, or —N(R^{6a})(R^{6b}).

10. The compound according to any of claims 1 to 5, or a pharmaceutically acceptable salt thereof,
wherein —Ar¹—(R¹)ₘ is a substituent represented by formula (3): 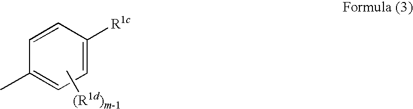
wherein R^{1c} represents —N(R^{6a})(R^{6b}); and
R^{1d} represents a substituent selected from the substituent group 1.

11. The compound according to any of claims 1 to 10, or a pharmaceutically acceptable salt thereof,
wherein at least one of R¹, the substituent of R¹, the substituent of R² selected from the substituent group 2, R⁵, and the substituent of R⁵ represents —COOH.

12. The compound according to any of claims 1 to 10, or a pharmaceutically acceptable salt thereof,
wherein the substituent of R² selected from the substituent group 2 represents —N(R^{6a})(R^{6b}) or —N(R^{6a})C(═NR^{6b})(NR^{6c}).

13. The compound according to any of claims 1 to 10, or a pharmaceutically acceptable salt thereof,
wherein at least one of R¹, the substituent of R¹, the substituent of R² selected from the substituent group 2, R⁵, and the substituent of R⁵ represents cyano.

14. The compound according to any of claims 1 to 5, or a pharmaceutically acceptable salt thereof,
wherein Ar¹ represents heteroaryl.

15. The compound according to any of claims 1 to 14, or a pharmaceutically acceptable salt thereof,
wherein Ar² represents C₆-C₁₀ aryl.

16. The compound according to any of claims 1 to 14, or a pharmaceutically acceptable salt thereof,
wherein Ar² represents heteroaryl.

17. A pharmaceutical composition comprising the compound according to any of claims 1 to 16, or a pharmaceutically acceptable salt thereof and a pharmaceutically acceptable carrier.

18. A cathepsin K inhibitor comprising the compound according to any of claims 1 to 16, or a pharmaceutically acceptable salt thereof as an active ingredient.

19. A drug comprising the compound according to any of claims 1 to 16, or a pharmaceutically acceptable salt thereof as an active ingredient for treatment or prevention of a disease selected from the group consisting of osteoporosis, osteoarthritis, chronic rheumatoid arthritis, Paget's disease of bone, hypercalcemia, bone metastasis of cancer, and ostealgia.
